(19) United States
(12) Patent Application Publication
(10) Pub. No.: US 2025/0102528 A1
(43) Pub. Date: Mar. 27, 2025

(54) METHODS OF ASSESSING CARCASS MUSCLE BIOMARKERS TO DISCRIMINATE CHARACTERISTICS OF THE MEAT

(71) Applicants: Daniel Silva ANTONELO, Pirassununga, São Paulo (BR); Julio Cesar BALIEIRO, Pirassununga, São Paulo (SE); PURDUE RESEARCH FOUNDATION, West Lafayette, IN (US); UNIVERSITY OF SAO PAULO, São Paulo (BR)

(72) Inventors: Christina Ramires FERREIRA, West Lafayette, IN (US); Daniel Silva ANTONELO, São Paulo (BR); Julio Cesar de Carvallho BALIERIO, São Paulo (BR)

(21) Appl. No.: 18/728,868

(22) PCT Filed: Jan. 13, 2023
(86) PCT No.: PCT/US23/60665

§ 371 Date: Jul. 13, 2024

Related U.S. Application Data
(60) Provisional application No. 63/299,061, filed on Jan. 13, 2022.

(51) Int. Cl. G01N 33/92; G01N 33/12

(52) U.S. Cl. CPC G01N 33/92; G01N 33/12; G01N 2560/00; G01N 2570/00

(57) ABSTRACT

Method of assessing biomarkers of a cattle carcass indicative of a characteristic of the animal such as finishing regimen or ultimate pH of the muscles of the animal, and kits to facilitate performing the method.

### PRIORITY

This patent application is related to and claims the priority benefit of U.S. Provisional Patent Application No. 63/299,061 filed Jan. 13, 2022. The content of the foregoing application is hereby incorporated by reference in its entirety into this disclosure.

### TECHNICAL FIELD

This disclosure relates to the analysis of animal carcass metabolites (e.g., muscle lipidome or muscle metabolome) to discern one or more characteristics of the animal or meat (e.g., feeding conditions, ultimate pH of the meat, gender of the animal, etc.), which can be used to assess the quality of commercial meat products for human consumption.

### BACKGROUND

Intramuscular fat (IMF), also known as marbling, is one of the most important meat quality traits. It affects quality grading, carcass price, and sensory acceptance of meat. Corbin et al., Sensory evaluation of tender beef strip loin steaks of varying marbling levels and quality treatments, Meat Sci 100:24-31 (2015); Silva et al., Explaining meat quality of bulls and steers by differential proteome and phosphoproteome analysis of skeletal muscle, J Proteomics 199:51-66 (2019).

Meat quality (e.g., beef quality) depends on many factors, one of which being the diet fed to the animal during life. A feeding regimen can impact animal growth rate and be related to changes in lipid metabolism, which, in turn, can directly impact subcutaneous and intramuscular fat composition, and consequently meat color, tenderness, and flavor. Mwangi et al., Diet and genetics influence beef cattle performance and meat quality characteristics, Foods 8:1-24 (2019); Wicks et al., Muscle energy metabolism, growth, and meat quality in beef cattle, Agric 9:1-10 (2019); Gagaoua et al., Clustering of sensory eating qualities of beef: Consistencies and differences within carcass, muscle, animal characteristics and rearing factors, Livest Sci 214:245-258 (2018); Hughes et al., Meat color is determined not only by chromatic heme pigments but also by the physical structure and achromatic light scattering properties of the muscle, Compr Rev Food Sci Food Saf 19:44-63 (2020); Valenzuela et al., Adipose invasion of muscle in Wagyu cattle: monitoring by histology and melting temperature, Meat Sci 163:108063 (2020). Thus, meat obtained from feed-lot finished cattle that are grain fed can differ significantly from meat obtained from pasture-finished cattle that forage for grass.

Currently, certifications of beef quality are based on on-site inspection and/or the completion of documentation (see, e.g., certifications available through the A Greener World organization). Conventional meat certifications and assurance schemes are not based on biomarkers, but rather are self-verification and the honor system. Labeling standards are also different depending on the animal.

In view of the above, a method of assessing animal carcasses is needed that is quick, precise, and simple to perform and provides a science-based approach to accurate verification of meat characteristics. Ideally, such method should be able to discriminate one or more aspects of animals' diet (e.g., the finishing regimen) even if the animals present different growth rates and age at slaughter. This and other objects and advantages, as well as inventive features, will be apparent from the detailed description provided herein.

### SUMMARY

A method of assessing an animal carcass for biomarkers indicative of one or more characteristics of the animal is provided. The characteristics can be, for example, a pH of the muscle, the gender of the animal, meat tenderness, and/or a finishing regime of the animal. The method can comprise obtaining a biomarker profile of a muscle sample from the animal carcass; comparing the biomarker profile to a representative aggregate biomarker profile of each of at least: (a) a first variation of a first characteristic, and (b) a second variation of the first characteristic; and based on the comparison, assessing the first characteristic of the animal carcass as the first variation or the second variation (e.g., by determinizing which representative aggregate biomarker profile the biomarker profile more accurately aligns). Obtaining the biomarker profile of the muscle sample can be performed in situ, for example, without prior extraction of the targeted biomarkers (e.g., lipids, small molecules, etc.) from the collected sample.

Obtaining the biomarker profile of the muscle sample can be performed using any analysis device capable thereof. In certain embodiments, the analysis device comprises a mass spectrometer. In certain devices, the analysis device comprises a portable mass spectrometer. In certain embodiments, obtaining the biomarker profile of the muscle sample does not require lipid extraction from the muscle sample, i.e., the biomarkers of interest are obtained from the muscle sample in situ and analyzed direction therefrom.

The method can further comprise grading the meat at least in part based on the assessed first characteristic of the animal carcass.

The animal can be any animal carcass (e.g., an animal carcass intended for food or consumption). In certain embodiments, the animal is poultry, bovine, porcine, ovine, or caprine. The one or more characteristics can be selected from the group consisting of pH of the muscle sample, gender of the animal, meat tenderness, and finishing regime of the animal.

The biomarker profile can be a lipidome profile. The biomarker profile can be a lipidome profile and the representative aggregate biomarker profile can be a representative aggregate lipidome profile comprising levels of specific diacylglycerols (DG), phosphatidylcholines (PC), triacylglycerols (TG), and sphingomyelins (SM).

In certain embodiments, the animal is bovine, the biomarker profile is a lipidome profile; the representative aggregate biomarker profile is a representative aggregate lipidome profile comprising levels of specific DG, PC, TG, and SM; the first characteristic is finishing regimen; and the first variation of the first characteristic is grass-finished cattle and the second variation of the first characteristic is grain-finished cattle.

The representative aggregate lipidome profile for grass-finished cattle can comprise: (i) increased levels of PC(32:0), PC(32:2), PC(36:1), PC(36:7), PC(36:8), PC(38:2), PC(32:4), SM(d18:1/22:0), SM(d18:1/24:0), TAG(16:0_36:1), TG (18:1_34:4), and TG (16:1_36:0) compared to a representative aggregate lipidome profile for grain-finished cattle, and (ii) decreased levels of DG (18:1_16:0), DG (18:1_18:1), PC(34:2), PC(34:3), PG (36:2), SM(d18:1/20:0), TG (16:0_32:3), and TG (18:0_34:2) compared to a representative aggregate lipidome profile for grain-finished cattle. The DG, PC, TG, and SM can comprise a total fatty acid chain length of at least 32 carbons and can have up to five sites of unsaturation in the fatty acyl chains. The DG and TG can comprise at least one palmitic (16:0) fatty acid, one palmitoleic (16:1) fatty acid, and one oleic (18:1) fatty acid, and the SM can be sphingosine ceramides comprising a d18:1 sphingoid base.

The representative aggregate lipidome profile for grass-finished cattle can further comprise a change in the level of one or more other PC, TG, DG and/or SM comprising palmitic (16:0) fatty acid, palmitoleic (16:1) fatty acid, and/or oleic (18:1) fatty acid, and/or a change in one or more other sphingosine ceramides comprising a d18:1 sphingoid base.

The first characteristic can be an ultimate pH (pHu) of the muscle sample. In certain embodiments, the biomarker profile is a lipidome profile comprising one or more specific PC, PE, SM, acyl-carnitine (AC), and triacylglycerides (TAG) content levels observed in the muscle at about 30 minutes postmortem; and the first variation of the first characteristic is muscle with normal pHu and the second variation of the first characteristic is muscle with high pHu.

The representative aggregate biomarker profile for the first variation (e.g., a muscle with high pHu) can comprise decreased levels of one or more of 56-carbon, unsaturated and monosaturated TAG as compared to the representative aggregate biomarker profile for the second variation (e.g., muscle with normal pHu). The representative aggregate biomarker profile of the second variation (e.g., muscle with normal pHu) can comprise increased levels of one or more of PC, PE, SM, and AC observed in the muscle at about 30 minutes postmortem as compared to such levels in the representative aggregate biomarker profile of the first variation (e.g., muscle with high pHu) observed in the muscle at about 30 minutes postmortem. In certain embodiments, the representative aggregate biomarker profile for the second variation (e.g., muscle with normal pHu) comprises increased levels of Lyso PC(18:3), PC(18:2), and PE (38:4) as compared to the representative aggregate biomarker profile for the first variation (e.g., muscle with high pHu).

In certain embodiments, the animal is bovine, the biomarker profile comprises a metabolome profile and the representative aggregate biomarker profile comprises a representative aggregate metabolome profile, the first characteristic is pH of the muscle sample; and the first variation of the first characteristic is muscle with normal pHu and the second variation of the first characteristic is muscle with high pHu. The representative aggregate metabolome profile can comprise levels of one or more of: (a) specific inosine monophosphate (IMP), creatinine, and creatine content observed in muscle at about 30 minutes postmortem, wherein the representative aggregate metabolome profile for muscle with high pHu can comprise increased levels of one or more of IMP, creatinine, and creatine content as compared to the representative aggregate metabolome profile for muscle with normal pHu; (b) glucose, glucose-1-phosphate, glucose-6-phosphate, and lactate content observed in muscle at about 30 minutes postmortem, wherein the representative aggregate metabolome profile for muscle with high pHu can comprise decreased levels of one or more of glucose, glucose-1-phosphate, glucose-6-phosphate, and lactate content as compared to the representative aggregate metabolome profile for muscle with normal pHu; and/or (c) adenosine, ADP, pyruvate, and an intermediate of oxidative metabolism content observed in muscle at about 44 hours postmortem, wherein the representative aggregate metabolome profile for muscle with high pHu can comprise increased levels of one or more of adenosine, ADP, pyruvate, and an intermediate of oxidative metabolism content as compared to the representative aggregate metabolome profile for muscle with normal pHu.

A method of generating a representative aggregate biomarker (e.g., lipidome or metabolome) profile for a first variation of a characteristic and/or a representative aggregate biomarker profile for a second variation of the characteristic is provided. In certain embodiments, such a method comprises analyzing, or having analyzed, (a) lipids or other metabolites obtained from muscle samples of a representative aggregate of carcasses of the first variation and/or (b) lipids or other metabolites obtained from muscle samples of a representative aggregate of carcasses of the second variation (e.g., wherein the carcasses of both the first and second variations are from the same species of animal such as, for example, bovine carcasses); whereupon a representative aggregate biomarker profile for animals of the first variation and/or a representative aggregate biomarker profile for animals of the second variation is/are generated.

The first variation can be grass-finished cattle and the second variation can be grain-finished cattle (e.g., wherein animal is bovine/cattle). The first variation can be meat having normal pHu and the second variation can be meat having high pHu.

In certain embodiments where the first variation is grass-finished cattle and the second variation is grain-finished cattle, the lipids or other metabolites obtained from muscle samples of a representative aggregate biomarker profile of animal (e.g., cattle) carcasses for grass-finished cattle can comprise levels of DG, phosphatidylcholines (PC), TG, and SM. The representative aggregate biomarker profiles of both the first and second variations can comprise PC(32:0), PC(32:2), PC(36:1), PC(36:7), PC(36:8), PC(38:2), PC(32:4), SM(d18:1/22:0), SM(d18:1/24:0), TG (16:0_36:1), TAG(18:1_34:4), TG (16:1_36:0), DG (18:1_16:0), DG (18:1_18:1), PC(34:2), PC(34:3), PG (36:2), SM(d18:1/20:0), TG (16:0_32:3), and TG (18:0_34:2). The representative aggregate biomarker profiles can further comprise one or more other PC, TG, DG and/or SM comprising palmitic (16:0) fatty acid, palmitoleic (16:1) fatty acid, and/or oleic (18:1) fatty acid, and/or one or more other sphingosine ceramides comprising a d18:1 sphingoid base.

In certain embodiments where the first variation is meat having normal pHu and the second variation is meat having high pHu, the lipids or other metabolites obtained from muscle samples of a representative aggregate of carcasses both the first and second variations comprise one or more specific PC, PE, SM, AC, and TAG content levels observed in the muscle at about 30 minutes postmortem.

Kits for assessing an animal carcass for biomarkers indicative of one or more characteristics of the animal are also provided. The kit can comprise a solution for retaining a sample obtained from a muscle of an animal carcass; a cartridge to deliver a retained sample to a device for analysis that results in a biomarker profile of the muscle sample, wherein the analysis does not require lipid (or other metabolite or small molecule) extraction from the retained sample; and, optionally, a representative aggregate biomarker profile of at least each of: (a) a first variation of a first characteristic, and (b) a second variation of the first characteristic. In certain embodiments, the representative aggregate biomarker profile of at least each of: (a) a first variation of a first characteristic, and (b) a second variation of the first characteristic can be external of the kit, for example, accessible online or via a local or remote database accessible by the user, or via an application (e.g., a mobile application, software-as-a-service application, or other software application). The cartridge can comprise a portion configured to receive the solution from the receptacle. The device for analysis can comprise a mass spectrometer.

The kit can further comprise a receptacle (e.g., that houses the solution). The kit can further comprise a tissue extraction device (e.g., a syringe, a biopsy device, or the like). In certain embodiments, the kit further comprises a receptacle that houses the solution, wherein the receptacle further comprises an extraction component for extracting the sample from the muscle of the animal (e.g., a needle, a biopsy device, a scalpel, etc.).

### DESCRIPTION OF THE DRAWINGS

The disclosed embodiments and other features, advantages, and aspects contained herein, and the matter of attaining them, will become apparent in light of the following detailed description of various exemplary embodiments of the present disclosure. Such detailed description will be better understood when taken in conjunction with the accompanying drawings.

FIG. 1A shows principal component (PC) analysis scores plot and heatmap analysis (top 25 most informative lipids of each class) of lipidomic distribution based on triacylglyceride (TG), and a listing of the classes analyzed in the heatmap analysis.

FIG. 1B shows PC analysis scores plot and heatmap analysis (top 25 most informative lipids of each class) of lipidomic distribution based on phosphatidylcholine (PC) phosphatidylethanolamine (PE), and sphingomyelin (SM), and a listing of the various classes analyzed in the heatmap analysis.

FIG. 1C shows PC analysis scores plot and heatmap analysis (top 25 most informative lipids of each class) of lipidomic distribution based on acyl-carnitine, ceramides (CER), diglyceride (DG), free fatty acids and phosphatidylglycerol (PG), phosphatidylinositol (PI) and phosphatidylserine (PS) between feedlot finished animals with high growth rate (F-H) and pasture finished animals with low growth rate (P-L).

FIG. 2A shows PC analysis scores plot and heatmap analysis (top 25 most informative lipids of each class) of lipidomic distribution based on TG.

FIG. 2B shows PC analysis scores plot and heatmap analysis (top 25 most informative lipids of each class) of lipidomic distribution based on phosphatidylcholine (PC), PE and SM.

FIG. 2C shows PC analysis scores plot and heatmap analysis (top 25 most informative lipids of each class) of lipidomic distribution based on acyl-carnitine, CER, DG, free fatty acids and PG, PI and PS between feedlot finished animals with low growth rate (F-L) and pasture finished animals with high growth rate (P-H).

FIG. 3A shows PC analysis scores plot and heatmap analysis (top 25 most informative lipids of each class) of lipidomic distribution based on TG.

FIG. 3B shows PC analysis scores plot and heatmap analysis (top 25 most informative lipids of each class) of lipidomic distribution based on phosphatidylcholine (PC), PE and SM.

FIG. 3C shows PC analysis scores plot and heatmap analysis (top 25 most informative lipids of each class) of lipidomic distribution based on acyl-carnitine, CER, DG, free fatty acids and PG, PI and PS between feedlot finished animals with high (F-H) and low (F-L) growth rate.

FIG. 4A shows PC analysis scores plot and heatmap analysis (top 25 most informative lipids of each class) of lipidomic distribution based on TG;

FIG. 4B shows PC analysis scores plot and heatmap analysis (top 25 most informative lipids of each class) of lipidomic distribution based on phosphatidylcholine (PC), PE and SM.

FIG. 4C shows PC analysis scores plot and heatmap analysis (top 25 most informative lipids of each class) of lipidomic distribution based on acyl-carnitine, CER, DG, free fatty acids and PG, PI and PS between feedlot finished animals with high (P-H) and low (P-L) growth rate.

FIG. 5 shows a plot of metabolite sets enrichment according to finishing regimen and growth rate.

FIG. 6 shows the top 24 lipids that were associated with beef color (L* and a*) and Warner-Bratzler shear force (WBSF) using Pearson's correlation as a distance measure.

FIG. 7A shows PC analysis scores plot and heatmap analysis (top 25 most informative lipids of each class) of lipid distribution based on Method 1-CER, phosphatidylcholine (PC), PE, PG, PI, and SM between longissimus thoracis muscle pHu classes (normal [<5.8] and high [≥6.2]) at 30 minutes postmortem.

FIG. 7B shows PC analysis scores plot and heatmap analysis (top 25 most informative lipids of each class) of lipid distribution based on Method 2-acyl-carnitine, DG, free fatty acids, and TAG between longissimus thoracis muscle pHu classes (normal [<5.8] and high [≥6.2]) at 30 minutes postmortem.

FIG. 8A shows a PC analysis scores plot and clustered heatmap analysis (top 25 most informative lipids of each class) of lipid distribution based on Method 1-CER, phosphatidylcholine (PC), PE, PG, PI and SM between longissimus thoracis muscle pHu classes (normal [<5.8] and high [>6.2]) at 44 hours postmortem.

FIG. 8B shows a PC analysis scores plot and clustered heatmap analysis (top 25 most informative lipids of each class) of lipid distribution based on Method 2-acyl-carnitine, DG, free fatty acids, and TAG between longissimus thoracis muscle pHu classes (normal [<5.8] and high [≥6.2]) at 44 hours postmortem.

FIGS. 9A and 9B show plots of enrichment analysis results according to longissimus thoracis muscle pHu classes (normal [<5.8] and high [≥6.2]) at 30 minutes postmortem (FIG. 9A) and 44 hours postmortem (FIG. 9B).

FIG. 10A shows PC analysis scores plot and heatmap analysis (top 25 most informative metabolites of each class) of longissimus thoracis muscle metabolome between pHu classes (normal [<5.8] and high [≥6.2]) at 30 minutes postmortem.

FIG. 10B shows PC analysis scores plot and heatmap analysis (top 25 most informative metabolites of each class) of longissimus thoracis muscle metabolome between pHu classes (normal [<5.8] and high [≥6.2]) at 44 hours postmortem.

FIGS. 11A and 11B show plots of pathway analysis of longissimus thoracis muscle metabolome between pHu classes (normal [<5.8] and high [≥6.2]) of 30 minutes postmortem (FIG. 11A) and 44 hours postmortem (FIG. 11B). The “metabolome view” presents pathways arranged according to the scores based on enrichment analysis (y-axis) and topology analysis (x-axis). The color and size of each circle are based on P-values and pathway impact values, respectively. All pathways described had P-values≤0.10 and pathway impacts≥0.10.

While the present disclosure is susceptible to various modifications and alternative forms, exemplary embodiments thereof are shown by way of example in the drawings and are herein described in detail.

### DETAILED DESCRIPTION

The present disclosure provides a method of assessing an animal carcass for one or more biomarkers (e.g., metabolites via, for example, a muscle lipidome, small molecules, etc.) to determine one or more characteristics of the animal and/or meat (e.g., beef, pork, lamb, goat, etc.).

The biomarkers can be indicative of quality characteristics of the meat, for example, a feeding regime of the animal, meat color, meat tenderness, and/or how quickly the meat will degrade. The biomarkers can be indicative of the sex, age, or gender of the animal. The biomarkers can be indicative of the conditions under which the animal was slaughtered. The animal can be poultry, bovine, ovine, caprine, or porcine (e.g., swine).

In certain embodiments, the method comprises comparing a biomarker profile for levels of specific metabolites in a sample taken from an animal carcass (e.g., a muscle) to a representative aggregate biomarker profile for the characteristic(s) being assessed and, based on the comparison, assessing the characteristic(s) of the carcass or muscle. In certain embodiments, the metabolites are a muscle lipidome. In certain embodiments, the metabolites are a muscle metabolome. In certain embodiments, the metabolites assessed does not include peptides.

In certain embodiments, the biomarkers (i.e., metabolites) assessed are small molecules and the characteristic is feed efficiency, animal slaughter conditions, gender of the animal, meat tenderness, and/or pH of the muscle. As used herein, “small molecule” means a molecule that has a molecular weight of at or less than about 1,500 daltons.

A “biomarker” means an expressed metabolite, transcript, small molecule, lipid, protein, polypeptide, or the like measurable in a subject or a sample taken from a subject. A biomarker can be differentially expressed in a subject that exhibits a particular characteristic or has experienced a particular characteristic as compared to the expression level of a control sample or the subject's baseline.

The increase or decrease, or quantification of biomarkers in a sample can be determined by any of several methods known in the art for measuring the presence and/or relative abundance of such biomarker and several specific (albeit nonlimiting) examples of such methods are described below. The level of biomarkers can be determined by an absolute value or a relative baseline value, and the level of a subject's biomarker(s) can be compared to a cutoff index. Alternatively, the relative abundance of the biomarker or biomarkers can be determined relative to a control (e.g., where the characteristic is slaughter conditions, the marker can be a small molecule and the control can be a relative abundance of the biomarker in an animal that was slaughtered and/or bled out in an unconscious and/or insensible condition).

The one or more characteristics to be assessed can be meat pH, meat tenderness, the gender of the animal, the age of the animal, the feeding regime, and/or the conditions under which the animal was slaughtered. In certain embodiments, multiple different characteristics can be assessed using the methods hereof, for example, both the pH of the animal's meat/muscle and its feeding regime. In certain embodiments, a characteristic can be determined from upregulation or downregulation of a relevant metabolic mechanism in a muscle, such as tyrosine and/or pyruvate pathways, which is measurable by assessment of one or more biomarkers. “Downregulation” and its variants refer to a decrease in the level of a biomarker as compared to an established level (e.g., that of a cohort of the subject of interest that exhibits or has experienced a known condition of the characteristic). “Upregulation” and its variants refer to an increase in the level of a biomarker as compared to an established level (e.g., that of a cohort of the subject of interest that exhibits or has experienced a known condition of the characteristic).

### Methods for Assessing Ultimate pH or Meat Tenderness of a Muscle

High pH beef can result from a stress-related decrease of muscle glycogen stores and attenuated postmortem metabolism following slaughter. Additionally, high meat pH can be linked to dark lean from “dark-cutting” cattle. Gagoua et al., Dark-cutting beef: A brief review and an integromics meta-analysis at the proteome level to decipher the underlying pathways, Meat Science 181: Article 108611 (2021); Ponnampalam et al., Causes and contributing factors to “dark cutting” meat: Current trends and future directions: A review, Comprehensive Reviews in Food Science & Food Safety 16 (3): 400-430 (2017). Changes in the muscle energy status at harvest, which can be altered by production practices such as feeding regime, age, and gender, can also result in a higher ultimate pH (pHu) of the meat. Dunne et al., Current perspectives on the darker beef often reported from extensively-managed cattle: Does physical activity play a significant role?, Livestock Science 142 (1-3): 1-22 (2011); Neethling et al., Exogenous and endogenous factors influencing color of fresh meat from ungulates, Meat & Muscle Biology 1 (1): 253-275 (2017); Wicks et al., Muscle energy metabolism, growth, and meat quality in beef cattle, Agriculture (Switzerland) 9 (9): 1-10 (2019).

In certain embodiments, a characteristic to be assessed can be the ultimate pH of the muscle (e.g., normal or high ultimate pH (pHu)), which can be indicative of how the meat will age and/or color over time. As used herein, “normal pHu meat” means an animal carcass or muscle having a pHu of 5.4 to 5.8, inclusive of the end points of the range, and “high pHu meat” means an animal carcass or muscle having a pHu of equal to or greater than 5.8. Consumer fresh purchasing decisions can be driven by lean color, with bright cherry-red appearance generally perceived as a positive indicator of meat freshness and wholesomeness. Suman & Joseph, Myoglobin chemistry and meat color, Animal Rev Food Science & Technology 4 (1): 79-99 (2013). Furthermore, studies show consumers are less interested in choosing darker beef, which generally exhibits very dark appearance even when the cut surface is exposed to extended periods of atmospheric oxygen. Ponnampalam et al. (2017), supra; McKeith et al., Mitochondrial abundance and efficiency contribute to lean color of dark cutting beef, Meat Science 116:165-173 (2016). As a result, carcasses with darker lean are generally downgraded during the process of grading. Given the value consumers place on fresh beef color in particular in making purchasing decisions and the variability in lean color known to exist in the beef industry worldwide, the ability to detect and accurately predict this highly coveted quality characteristics in fresh beef would be greatly beneficial. At a minimum, for example, it would provide the industry with the ability to minimize the occurrence of this quality aberration or facilitate an understanding of how beef quality development can be controlled postmortem.

Biochemically, dark meat is characterized by the reduction in metabolites involved in glycolytic pathways and an increase in tricarboxylic acid (TCA) cycle metabolites, which are indicative of mitochondrial activity and can decrease the amount of available oxygen in the tissues. Cônsolo et al., Preliminary study on the characterization of longissimus lumborum dark cutting meat in Angus×Nellore crossbreed cattle using NMR-based metabolomics, Meat Science 172: Article 108350 (2021); Ramanathan et al., Biomolecular interactions governing fresh meat color in post-mortem skeletal muscle: A review, J Agricultural & Food Chemistry 68 (46): 12779-12787 (2020). Additionally, it is suggested that mitochondria from darker lean may use substrates other than carbohydrates for oxidative phosphorylation in an attempt to preserve energy homeostasis in muscle tissue postmortem. This can be leveraged using the methods hereof to determine characteristics of the animal and/or meat. For example, as set forth in the below Examples, the presence of certain lipids such as phosphatidylcholine (PC), phosphatidylethanolamine (PE), sphingomyelin (SM), and acyl-carnitine (AC) can positively correlate with beef lightness (L*), while other metabolites associated with lipid biosynthesis, such as triacylglycerides (TAG) and AC negatively correlate with redness (a*), though phosphatidylglycerols (PG) can positively correlate with a*.

In certain embodiments, a method of assessing an animal carcass (e.g., a muscle) for biomarkers indicative of one or more characteristics (e.g., a pHu of the muscle or meat tenderness) is provided. The method can comprise obtaining a biomarker profile of a muscle sample taken from an animal carcass and comparing the biomarker profile to a representative aggregate biomarker profile of at least two variations (e.g., a first variation and a second variation) of a first characteristic (e.g., muscle pHu or meat tenderness, in which cases the first variation can be muscle with high pHu or muscle that rates as tough using standard methods and the second variation can be muscle with normal pHu or muscle that rates as tender using standard methods, respectively). Based on the comparison, the first characteristic of the animal carcass can be assessed/assigned as either the first variation or the second variation.

For example, where the first characteristic is muscle pHu, the first variation is normal pHu and the second variation is high pHu, the method can comprise obtaining a biomarker profile of a muscle sample from an animal carcass; comparing the biomarker profile to 1) a representative aggregate biomarker profile of carcasses of normal pHu, and 2) a representative aggregate biomarker profile of carcasses of high pHu; and, based on the comparison, assessing the muscle pHu of the animal carcass as normal pHu (e.g., if the biomarker profile better aligns with the representative aggregate biomarker profile of animal carcasses of normal pHu as compared to the representative aggregate biomarker profile of animal carcasses of high pHu) or as high pHu (e.g., if the biomarker profile better aligns with the representative aggregate biomarker profile of animal carcasses of high pHu as compared to the representative aggregate biomarker profile of animal carcasses of normal pHu).

The method can further comprise grading the meat at least in part on the assessed characteristic (e.g., meat tenderness or meat pHu).

The biomarker profile used for assessing a pHu of a muscle can be a lipidome profile. The biomarker profile used for assessing a pHu of a muscle can be a metabolome profile. In certain embodiments, the biomarker profile for assessing a pHu of a muscle can be both a lipidome and metabolome profile.

The lipidome profile can comprise one or more of specific PC, PE, SM, AC, and TAG content levels observed in the muscle, for example, at about 30 minutes postmortem. In certain embodiments, the representative aggregate biomarker profile for muscle with high pHu (e.g., the first variation of the first characteristic) comprises decreased levels of one or more of 56-carbon, unsaturated and monosaturated TAG as compared to the representative aggregate profile for muscle with normal pHu (e.g., the second variation of the first characteristic). In certain embodiments, the representative aggregate profile for muscle with high pHu (e.g., the first variation of the first characteristic) can comprise increased levels of one or more of PC, PE, SM, and AC as compared to the representative aggregate biomarker profile for muscle with normal pHu (e.g., the second variation of the first characteristic). In certain embodiments, the representative aggregate biomarker profile for muscle with high pHu (e.g., the first variation of the first characteristic) can comprise increased levels of PC, PE, SM, and AC as compared to the representative aggregate biomarker profile for muscle with normal pHu (e.g., the second variation of the first characteristic).

The metabolome profile can comprise one or more of specific IMP, creatinine, and creatine content (e.g., observed in muscle at about 30 minutes postmortem), one or more of glucose, glucose-1-phosphate, glucose-6-phosphate, and lactate content (e.g., observed in muscle at about 30 minutes postmortem), and/or one or more of adenosine, ADP, pyruvate, and an intermediate of oxidative metabolism content (e.g., observed in muscle at about 30 minutes postmortem). The representative aggregate biomarker profile for muscle with high pHu (e.g., the first variation of the first characteristic) can comprise increased levels of one or more of IMP, creatinine, and creatine as compared to the representative aggregate biomarker profile for muscle with normal pHu (e.g., the second variation of the first characteristic). In certain embodiments, the representative aggregate biomarker profile for muscle with high pHu can comprise increased levels of IMP, creatinine, and creatine as compared to the representative aggregate biomarker profile for muscle with normal pHu.

The representative aggregate biomarker profile for muscle with high pHu (e.g., the first variation of the first characteristic) can comprise decreased levels of one or more of glucose, glucose-1-phosphate, glucose-6-phosphate, and lactate content as compared to the representative aggregate biomarker profile for muscle with normal pHu (e.g., the second variation of the first characteristic). In certain embodiments, the representative aggregate biomarker profile for muscle with high pHu can comprise decreased levels of glucose, glucose-1-phosphate, glucose-6-phosphate, and lactate content as compared to the representative aggregate biomarker profile for muscle with normal pHu.

The representative aggregate biomarker profile for muscle with high pHu (e.g., the first variation of the first characteristic) can comprise increased levels of one or more of adenosine, ADP, pyruvate, and an intermediate of oxidative metabolism content as compared to the representative aggregate profile for muscle with normal pHu (e.g., the second variation of the first characteristic). In certain embodiments, the representative aggregate biomarker profile for muscle with high pHu can comprise increased levels of adenosine, ADP, pyruvate, and an intermediate of oxidative metabolism content as compared to the representative aggregate biomarker profile for muscle with normal pHu.

Lipid and metabolic profiles of muscle can also be used to determine characteristics associated with pHu, which can then be used to provide insight into why and/or if some meat is/will be darker than others. For example, the methods hereof can be utilized in conjunction with the meat grading process, with a carcass assessed as having a high pHu being generally downgraded as it will result in darker lean, and/or a carcass assessed as having a normal pHu being indicative of a lean, red color of the meat.

A method of generating a representative aggregate biomarker profile for normal pHu muscle and/or a representative aggregate profile for high pHu muscle is also provided. The method comprises analyzing, or having analyzed, (a) lipids or other metabolites obtained from muscle samples of a representative aggregate of animal carcasses having normal pHu meat and/or (b) lipids or other metabolites obtained from muscle samples of a representative aggregate of animal carcasses having high pHu meat for levels of AC, TAG, PC, PE, SM, IMP, creatinine, creatine, glucose, glucose-1-phosphate, glucose-6-phosphate, lactate, adenosine, ADP, pyruvate, and/or another intermediate of oxidative metabolism content. The animal carcasses assessed for both the first and second variations (e.g., normal pHu and high pHu, respectively) can be from animals of the same species. The representative aggregate biomarker profiles can comprise, for example, one or more 56-carbon, unsaturated and monosaturated TAG. The representative aggregate profile can further comprise one or more AC, TAG, PC, PE, SM, comprising Lyso PC(18:3), PC(40:8), PE (18:2), PE (36:3), and PE (38:4).

Methods for Discriminating Feeding and/or Finishing Regimens

A characteristic to be assessed can be the feeding regimen of the animal. In certain embodiments, a method of assessing an animal carcass (e.g., a muscle) for biomarkers indicative of one or more characteristics (e.g., a feeding regimen of the animal) is provided. The method can comprise obtaining a biomarker profile of a muscle sample taken from the animal carcass and comparing the biomarker profile to a representative aggregate biomarker profile of at least a first variation of a first characteristic (e.g., grass-fed regimen) and a second variation of the first characteristic (e.g., grain-fed regimen). Based on the comparison, the first characteristic of the animal carcass can be assessed/assigned as either the first variation or the second variation.

In addition to grass-fed versus grain-fed finishing regimens, the methods hereof can also be utilized to assess if an animal's feeding regimen in life consisted of other animals' processed parts, which is a practice that is regulated and/or banned in multiple jurisdictions. The feeding of animal by-products and parts to another animal can be reflected in the muscle lipidome and/or metabolome of the animal and, as such, the present methods can be employed to not only detect such feeding practices, but also to monitor emerging disease that may result therefrom.

The lipid composition of a muscle from a carcass can be influenced by the nutrition the animal was fed during life. Indeed, significant physiological differences can be observed in meat coming from a grain-finished animal versus a pasture-finished animal. Accordingly, the muscle lipidome of an animal carcass can be assessed for quality characteristics indicative of a feeding regimen during finishing. “Finishing” is a term used to indicate a period of growth of usually 3-10 months when animals, which have reached puberty, become physically mature in skeleton and muscle and accumulate fatty tissue on the exterior of the muscle (fat cover) and in the interior of the muscle (“marbling”). Much of the flavor and tenderness of the meat will be related to its fat content.

The biomarker profile used for assessing a feeding regimen of an animal can be a lipidome profile. In certain embodiments, the metabolites assessed are a muscle lipidome.

Using beef as a non-limiting example, in general, beef calves are traditionally weaned between 6-8 months of age and then reared on forages and finished on high-energy diets containing grains. The total time for an animal to reach physical maturity and develop exterior and intramuscular fat typically takes 12-36 months (depending on the production system), of which the last 3-10 months are the finishing period. In the Examples set forth below, beef calves were weaned at 8 months, reared on forages until 12 months, and then finished either in a feedlot system (i.e., a grain-based diet, an example of what is referred to herein as “grain-finished”) or an extensive system (i.e., a pasture- or forage-based diet, an example of what is referred to herein as “grass-finished”) until reaching a weight of 530 kg, at which time the animals were slaughtered. Thus, “grass-finished” means the animal was reared and finished in an extensive system (a pasture- or forage-based diet). In contrast, “grain-finished” means the animal was reared on a grass-based diet, and then finished in a feedlot system (a grain-based diet).

In certain embodiments, the method comprises assessing a muscle lipidome of a carcass for biomarkers indicative of a finishing regimen (i.e., the characteristic of the meat). Being able to quickly and accurately identify the finishing regimen of an animal carcass can be of significant commercial import as there is a growing market for meats that have been finished in a particular manner. For example, many consumers seek beef labeled as “grass-fed,” which is often offered at a different price point than “grain-fed” beef. However, there is currently little regulation behind what meat can be labeled and offered as “grass-fed.”

The present methods provide a quick and accurate process that can provide more consistent verification of the finishing regimen undergone by the animal. Such verification can be used for certifying the resulting meat product (e.g., to facilitate controls and/or the accuracy of grass-grain fed beef certifications). In certain embodiments, the method further comprises grading the meat at least in part on the assessed characteristic(s).

The method comprises obtaining a lipidome profile of a muscle sample from the cattle carcass, comparing the lipidome profile for levels of specific diacylglycerols (DG), phosphatidylcholines (PC), triacylglycerols (TG), and sphingomyelins (SM) to a representative aggregate lipidome profile for grass-finished cattle and/or a representative aggregate lipidome profile for grain-finished cattle, and, based on the comparison, assessing the quality characteristics of the cattle carcass as grass-finished or grain-finished.

The representative aggregate lipidome profile for grass-finished cattle can comprise (i) increased levels of PC(32:0), PC(32:2), PC(36:1), PC(36:7), PC(36:8), PC(38:2), PC(32:4), SM(d18:1/22:0), SM(d18:1/24:0), TAG(16:0_36:1), TG (18:1_34:4), and TG (16:1_36:0) compared to a representative aggregate lipidome profile for grain-finished cattle, and (ii) decreased levels of DG (18:1_16:0), DG (18:1_18:1), PC(34:2), PC(34:3), PG (36:2), SM(d18:1/20:0), TG (16:0_32:3), and TG (18:0_34:2) compared to a representative aggregate lipidome profile for grain-finished cattle. The DG, PC, TG, and SM can comprise a total fatty acid chain length of at least 32 carbons and can have up to five sites of unsaturation in the fatty acyl chains. The DG and TG can comprise at least one palmitic (16:0) fatty acid, one palmitoleic (16:1) fatty acid, and one oleic (18:1) fatty acid, and the SM can be sphingosine ceramides comprising a d18:1 sphingoid base. The representative aggregate lipidome profile for grass-finished cattle can further comprise (i) a change in the level of one or more other PC, TG, DG and/or SM comprising palmitic (16:0) fatty acid, palmitoleic (16:1) fatty acid, and/or oleic (18:1) fatty acid, and/or (ii) a change in one or more other sphingosine ceramides comprising a d18:1 sphingoid base.

A method of generating a representative aggregate lipidome profile for grass-finished cattle and/or a representative aggregate lipidome profile for grain-finished cattle is also provided. The method comprises analyzing, or having analyzed, (a) lipids obtained from muscle samples of a representative aggregate of cattle carcasses for grass-finished cattle and/or (b) lipids obtained from muscle samples of a representative aggregate of cattle carcasses for grain-finished cattle for levels of DG, PC, TG and SM. The representative aggregate lipidome profile can comprise PC(32:0), PC(32:2), PC(36:1), PC(36:7), PC(36:8), PC(38:2), PC(32:4), SM(d18:1/22:0), SM(d18:1/24:0), TG (16:0_36:1), TAG(18:1_34:4), TG (16:1_36:0), DG (18:1_16:0), DG (18:1_18:1), PC(34:2), PC(34:3), PG (36:2), SM(d18:1/20:0), TG (16:0_32:3), and TG (18:0_34:2). The representative aggregate lipidome profile can further comprise one or more other PC, TG, DG and/or SM comprising palmitic (16:0) fatty acid, palmitoleic (16:1) fatty acid, and/or oleic (18:1) fatty acid, and/or one or more other sphingosine ceramides comprising a d18:1 sphingoid base.

### Muscle Samples and Biomarker Analysis

For the method thereof, samples can be obtained from any suitable muscle from an animal carcass, such as a muscle which is part of a cut of meat which is typically sold at market, such as for human consumption, and/or is easily accessible for rapid collection. To minimize variation in analysis between animals, samples can be taken from the same muscle (e.g., longissimus thoracis) from each carcass undergoing analysis. Samples can be removed immediately after slaughter up to about 24 hours postmortem or about 44 hours postmortem. Samples can be obtained in accordance with any suitable method known in the art.

Samples can be analyzed immediately post-slaughter. Alternatively, samples can be refrigerated short-term, e.g., minutes to hours, at around 4° C. Samples that require storage for longer periods of time prior to analysis can be frozen, e.g., stored at −80° C., to prevent degradation of the metabolites of interest and/or the meat itself.

Samples can be analyzed on-site, i.e., at an abattoir. In certain embodiments, an assay is provided for assessing metabolites (e.g., a muscle lipidome, muscle metabolome, small molecules, etc.) in an animal carcass to determine one or more characteristics of the meat using the methods hereof.

The assay can be performed in minutes (e.g., about two minutes) and can be adapted to miniaturized instrumentation. Instrumentation can include, but is not limited to, a benchtop and/or a miniaturized or portable mass spectrometer. The spectrometer can have small molecules ionized by electrospray or by a direct ionization method that does not include lipid extraction but allows lipids and metabolites to be detected in situ. Alternatively, the spectrometer can be operated in another commercial setting, such as a centralized laboratory to which samples are shipped in refrigerated containers and results are reported out.

Any suitable method of analysis (e.g., lipid or metabolite analysis) can be used as known in the art. An example of such a method is multiple reaction monitoring (MRM) profiling. MRM profiling is a mass spectrometry-based method for small molecule profiling that enables accelerated discovery of a large number of discriminating molecular features. Xie et al., Multiple reaction monitoring profiling (MRM profiling): Small molecule exploratory analysis guided by chemical functionality, Chem Phys Lipids 235:105048 (2021).

### Kits

Kits for performing the methods are also provided. In certain embodiments, a kit is provided for assessing an animal carcass for biomarkers indicative of one or more characteristics of the animal (or meat thereof). Such kit comprises a solution to retain a sample obtained from a muscle of an animal carcass; a cartridge to deliver a retained sample to a device for analysis that results in a biomarker profile of the muscle sample (e.g., where the analysis is performed in situ without the need for lipid extraction from the retained sample); and a representative aggregate biomarker profile of at least each of (a) a first variation of a first characteristic; and (b) a second variation of the first characteristic.

The characteristics and variations thereof can be any described herein or otherwise contemplated in view of the present disclosure. The representative aggregate biomarker profiles can be any of those described herein or contemplated in view of the present disclosure. The representative aggregate biomarker profiles can be written, provided in digital or other data formats, or accessible via a link or other indicator provided in the kit (e.g., a QR code, URL address, or other link to where the information can be obtained) that facilitates access to where such representative aggregate biomarker profiles are stored in a database such as, for example, on the Internet or in an accessible database. In certain embodiments, the kit can comprise a link to download an application configured to provide the desired representative aggregate biomarker profiles. In certain embodiments, the kit need not comprise the representative aggregate biomarker profiles; instead, a user can access the desired profiles via an application (e.g., a mobile application, a locally stored software application, a software-as-a-service application, and the like). In certain embodiments, such an application can receive the data from the analysis device and automatically compare the results to the desired representative aggregate profiles to determine the characteristic(s) of the meat sample.

The kit can further comprise a receptacle (e.g., a vial, tube, etc.) that houses the solution. The solution can be an aqueous solution at a substantially neutral pH (i.e., pH from 4-9). In certain embodiments, the solution can be formulated to prevent degradation of one or more molecules (i.e., biomarkers) of interest that are or may be present within a retained sample. The receptacle can be prefilled with a stable formulation for retaining and preserving the collected sample.

The kit can further comprise a syringe or extraction device. The syringe or extraction device can comprise a needle and/or comprise a soft tissue biopsy device. The extraction device can comprise a biopsy device with integrated vacuum technology as is known in the art.

In certain embodiments, the receptacle for retaining the solution can further comprise a syringe or extraction component such that when the sample is extracted, it is directly retained within the receptacle. The syringe or extraction component can be integral with or separate (and, for example, attachable to) the receptacle. In certain embodiments, the extraction device is a syringe comprising a needle (or another biopsy device), a barrel, and a plunger. The barrel of the syringe can comprise the receptacle of the kit and the solution can be prefilled therein. Alternatively, the receptacle can comprise a removable cap for placement over an opening of the receptacle, and the kit can further comprise an extraction component that seats over an opening in the receptacle when the cap is removed. Suction can be applied through the tip of the extraction component to extract and retain the sample of muscle by squeezing walls of the attached receptacle or as is otherwise known in the art.

The cartridge can be any cartridge known in the art or hereinafter developed that is suitable for delivering the collected sample to the analysis device (e.g., a portable mass spectrometer). The cartridge can comprise a portion configured to receive the solution from the receptacle, wherein the solution comprises the sample.

The kits can further comprise instructions. For example, such instructions can direct the extraction of a sample from an animal carcass, the retention of the collected sample within the receptacle, the delivery of the collected sample to the cartilage, insertion of the cartilage into an analysis device, and/or operation of the analysis device (e.g., a portable mass spectrometer).

### EXAMPLES

The following examples serve to illustrate the present disclosure. The examples are not intended to limit the scope of the claimed invention in any way.

### Example 1

### Materials & Methods for Lipidome Studies

Thirty-six contemporary ½ Angus×½ Nellore crossbred steers (330±30 kg body weight (BW); 12±1 months-old) were subjected to a completely randomized experimental design consisting of four treatments (n=9 per treatment) as shown in Table 1:

TABLE 1
Treatment Design

| | Projected | Time to reach | |
|---|---|---|---|
| | average daily | BW mean of 530 kg | |
| Treatment | gain (ADG) | for harvest | Actual ADG |
| Feedlot finished, | 1.5 kg/day | 116 days | 1.51 kg/day |
| high growth rate | | | |
| (F-H) | | | |
| Feedlot finished, | 0.9 kg/day | 228 days | 0.94 kg/day |
| low growth rate | | | |
| (F-L) | | | |
| Pasture finished, | 0.9 kg/day | 262 days | 0.76 kg/day |
| high growth rate | | | |
| (P-H) | | | |
| Pasture finished, | 0.6 kg/day | 292 days | 0.62 kg/day |
| low growth rate | | | |
| (P-L) | | | |

Steers were fasted for 16 hours and then transported (1-4 km) for harvest. After carcasses were chilled for 24 hours at 0-2° C., samples were excised from the longissimus thoracis (LT) muscle at the 12th and 13th rib level for pH, total intramuscular fat (IMF), and lipidomic analysis. All samples were snap frozen in liquid nitrogen and stored at −80° C. One 2.5-cm thick LT sample between the 12th and 13th ribs was collected from each color for beef color and shear force instrumental analyses.

Instrumental color. Fresh samples (n=9 per treatment) were exposed to air for 30 minutes at 4° C. to bloom (Meat color measurement guidelines. American Meat Science Association. Champaign, IL, USA (2012)). The meat color was evaluated using the CIE Lab system (Official recommendations on uniform colour space, colour difference equations and metric colour terms. Publication no. 15 (E-1.3.1)., Suppl. 2nd ed. Commission Internationale de l'Eclairage, Paris, France. (1979)) with a portable spectrophotometer model CM2500d (Konica Minolta Brazil, Sao Paulo, Brazil) with standard illuminant D65, a 10° observation angle, and a 30 mm aperture (AMSA, 2012). L* (lightness), a* (redness), and b* (yellowness) values were determined by averaging three measurements per sample.

Instrumental shear force. After color measurement, steaks were roasted in an oven equipped with a thermostat adjusted to 170° C. (Model F130/L-Electric Furnaces Golden Arrow Industry and Commerce Ltda., Sao Paulo, Brazil). Internal meat temperatures were monitored using individual thermometers. Once steaks reached an internal temperature of 40° C., steaks were turned and cooked to an internal temperature of 71° C., as recommended by the American Meat Society Association (Research guidelines for cookery, sensory evaluation, and instrumental tenderness measurements of meat. 2nd ed. American Meat Science Association, Champaign, IL, USA (2015)). Steaks were then cooled to 4° C. for 12 hours and six 1.27 cm diameter core samples were taken parallel to muscle fiber orientation using a Drill Bench (Model FG-13B, Caracol Trading of Machinery and Tools LTDA, Sao Paulo, Brazil). Shear force was determined on cores using the TMS-PRO texture analyzer (Food Technology Corporation, Sterling, Virginia, USA) coupled with a Warner-Bratzler shear device that was set at a speed of 200 mm/min (AMSA (2015), supra). Shear force values were determined by averaging the maximum peak force of six cores per sample.

Total intramuscular fat (IMF) analysis. The lipids were extracted by homogenizing (Ultra-Turrax®, T 25 digital, IKA, Campinas, SP, Brazil) the muscle samples (n=9 per treatment) with a chloroform:methanol:distilled water (2:1:0.8; v/v/v) solution, and the total IFM was determined by gravimetry, according to a method reported by Bligh & Dyer, A rapid method of total lipid extraction and purification, Can J Biochem Physiol 37:911-917 (1959).

### Example 2

### Meat Quality Analysis

Steaks prepared in Example 1 from F-H animals had higher L* (P<0.001; lighter appearance), a* (P=0.002; increased redness), and b* (P<0.001; increased yellowness) than those from other treatments (Table 2). Steaks from F-H also had higher IMF (P=0.001) deposition than P-H and P-L animals, but no differences when compared to F-L animals (P>0.05). F-L animals had higher L* (P<0.05) and similar IMF (P>0.05) than P-H and P-L animals, which did not differ for beef quality traits. In addition, P-H and P-L animals had higher Warner-Bratzler shear force (WBSF) (less tender) than F-H and F-L animals (P<0.05).

TABLE 2
Meat Analysis Results

| Trait | F-H | F-L | P-H | P-L | P-value |
|---|---|---|---|---|---|
| Lightness | 40.3^{a} ± 0.72 | 37.0^{b} ± 0.72 | 35.0^{c} ± 0.67 | 34.7^{c} ± | <0.001 |
| (L*) | | | | 0.62 | |
| Redness | 19.3^{a} ± 0.55 | 16.9^{b} ± 0.55 | 16.6^{b} ± 0.51 | 16.4^{b} ± | 0.002 |
| (a*) | | | | 0.47 | |
| Yellowness | 15.9^{a} ± 0.51 | 12.8^{b} ± 0.51 | 12.4^{b} ± 0.48 | 12.2^{b} ± | <0.001 |
| (b*) | | | | 0.45 | |
| Shear force | 60.6^{b} ± 4.32 | 64.2^{b} ± 4.32 | 86.0^{a} ± 4.00 | 78.1^{a} ± | 0.004 |
| (N) | | | | 3.74 | |
| IMF (%) | 6.06^{a} ± 0.76 | 4.95^{ab} ± 0.76 | 3.14^{b} ± 1.07 | 2.27^{b} ± | 0.001 |
| | | | | 1.07 | |

^{a-c}Values within a row with different superscripts differ significantly at P < 0.05.

Feeding regimen and growth rate impacted IMF deposition; F-H animals had higher IMF deposition than P-L animals (see Table 2). Under a similar growth fate, F-L and P-H animals had similar IMF deposition, suggesting diet is the main driver of IMF deposition. Therefore, feeding regimen or dietary component (grain vs. grass) mainly alters IMF deposition.

### Example 3

### Feeding Regime Lipidome Analysis

Six samples per treatment in Example 2 were randomly selected, and approximately 50 mg of each sample were weighed and ground in liquid nitrogen for lipid extraction using a method reported by Bligh & Dyer (1959), supra. Tissue homogenate (300 μL in ultrapure water) was transferred to a new microtube and mixed with 250 μL of chloroform and 450 μL of methanol. This solution was incubated at 4° C. for 15 minutes prior to the addition of 250 μL of chloroform and 250 μL of water and centrifugation for 10 minutes at 16,000×g, forming a two-phase solution where the bottom phase contained the lipids (organic phase). The organic phase was transferred to a new tube and dried using a centrifugal vacuum concentrator (Genevac™ miVac, Genevac LTD., Ipswich, UK), and samples were stored at −80° C. until further analysis.

Targeted lipid profiling was performed using discovery multiple reaction monitoring (MRM)-profiling methods and instrumentation as recently reviewed by Xie et al. (2021), supra. Specifically, dried lipid extracts were diluted in 50 μL of methanol/chloroform 3:1 (v/v) and 250 μL of injection solvent (acetonitrile/methanol/ammonium acetate 300 mM 3:6.65:0.35 [v/v/v]) to obtain a stock solution. Mass spectrometry data were acquired by flow-injection (no chromatographic separation) from 8 μL of stock solution that was diluted 300× in injection solvent that was spiked with EquiSPLASH™ LIPIDOMIX® Quantitative Mass Spec Internal Standard (0.1 ng/μL of each of the internal standards) prior to being delivered using a micro-autosampler (G1377A) to the ESI source of an Agilent 6410 triple quadrupole mass spectrometer (Agilent Technologies, Santa Clara, CA, USA). A capillary pump was connected to the autosampler and operated at a flow rate of 7 μL/min and pressure of 100 bar. Capillary voltage of the instrument was 4 kV, and gas flow was 5.1 L/min at 300° C.

The MRM profiling method was used to profile 1,366 MRMs related to lipids, from 11 lipid classes. The MRM set included 152 phosphatidylethanolamines (PE), 62 acyl-carnitines (AC), 57 cholesteryl esters (CE), 121 phosphatidylcholines (PC), 27 sphingomyelins (SM), 148 phosphatidylinositols (PI), 152 phosphatidylglycerols (PG), 148 phosphatidylserines (PS), 36 free fatty acids (FFA), 80 ceramides (CER), and 383 triacylglycerols (TG). TGs were profiled using parent ions and a product ion related to the presence of specific fatty acyl residues (C16:0, C16:1, C18:0, C18:1, C18:2, and C20:4). The assigned short-hand identity, with TG 16:0_36:1 as an example, starts with class (TG) followed by the fatty acyl chain related to the product ion (16:0) and ends with the sum of the carbon: unsaturation number related to the other two fatty acyl chains at the TG (e.g., 36:1), as recommended by the Lipid Maps nomenclature group. Liebisch et al., Update on LIPID MAPS classification, nomenclature, and shorthand notation for MS-derived lipid structures, J Lipid Res 61:1539-1555 (2020).

Phospholipids were identified by their class (PG, PS, PI, PE, or PC), the number of carbon atoms between both esterified fatty acids, and the number of carbon-carbon double bonds present in the molecule, e.g., PE (34:4). Ion intensity data of each MRM per sample were obtained using in-house scripts that were used for subsequent analysis.

Meat quality data were analyzed in a completely randomized design considering treatments (F-H, F-L, P-H, and P-L) as fixed effects, and the animals as experimental units. Data were analyzed using the PROC MIXED procedure of SAS 9.4 (SAS Institute Inc., Cary, NC). The least square means (LSMEANS) statement was used to calculate the adjusted means for treatment, and the means were compared by Student's t test. Differences were considered statistically significant when P≤0.05 and marginally significant when 0.05<P≤0.10.

To avoid noisy MRMs, ion intensities of 1.3-fold or higher than the ion intensity for a blank sample were considered for statistical analysis. Relative ion intensity was calculated for each MRM by dividing its ion intensity by the sum of all ion intensities across the sample. MRMs were then assigned to one of three lipid classifications: A) TG; B) PC, PE, and SM; and C) AC, CER, DG, FFA, PG, PI, and PS. Using these designations, the following comparisons were made: 1) F-H vs. P-L, which simulates differences between traditional beef production systems; 2) F-L vs. P-H, which reflects the effect of nutrient type on lipid deposition; and 3) F-H vs. F-L and P-H vs. P-L, which examines the effect of growth rate on lipid deposition. The effect of treatments on TG distribution in samples was analyzed using three distinct groupings for Student's t-test analysis: 1) total number of carbons [e.g., TG (54)]; 2) total number of sites of unsaturation [e.g. (: 4)]; and 3) total number of sites of unsaturation grouped into 0, up to 2, and more than 3. The relative ion intensities were uploaded to Metaboanalyst 5.0. Chong et al., Using MetaboAnalyst 4.0 for Comprehensive and Integrative Metabolomics Data Analysis, Current Protoc. Bioinforma. 68:1-128 (2019). Relative ion intensity data were normalized by auto-scaling, and statistical analysis was performed using Student's t-test. Moreover, the information of the internal standard was used to obtain the relative quantification of the total TG, total phospholipid, and phospholipid profile according to its class. Differences were considered statistically significant when P≤0.05 and marginally significant when 0.05<P≤0.10.

Principal component analysis (PCA) was performed, heatmaps were developed using the differentially abundant lipids for each sample type, and Ward's method was used to cluster samples. Quantitative enrichment analysis was performed using lipid quantification data sets for each treatment. The compound name was standardized according to KEGG, HMDB or PubChem ID, and the library chosen was the small molecule pathway database (SMPDB). Correlation analysis was performed between beef quality traits and lipid compounds using the PatternHunter method with the Pearson's correlation applied as a distance measure.

Of the 1,366 ion transitions (MRMs) scanned, which are tentatively attributed at the lipid species level, 440 were found to have an intensity of at least 1.3-fold higher than the blank sample (injection solvent). These were related mainly to TG (137) and PC(100) lipids.

Relative ion intensity data showed that 107 TG, 39 PC, 8 SM, 6 PE, 5 DG, 3 AC, 2 PG, 1 CER, and 1 CE were differentially abundant (P<0.05) between F-H and P-L animals (see Table 3). There were 51 PC, 10 SM, 9 AC, 9 PE, 9 TG, 4 PI, 3 DG, and 2 FFA that were differentially abundant (P<0.05) between F-L and P-H animals (see Table 4), while there were 80 TG, 15 PE, 14 PC, 7 SM, and 1 DG differentially abundant (P<0.05) lipids between F-H and F-L animals (see Table 5). Moreover, 38 PC, 17 PE, 8 AC, 8 SM, 7 TG, 1 CE, 1 DG, 1 FFA, and 1 PS were differentially abundant (P<0.05) between P-H and P-L animals (see Table 6).

TABLE 3
F-H and P-L Animal Data

| | | | | | |
|---|---|---|---|---|---|
| Lipid, % | F-H | P-L | Fold ratio^{a} | SEM | P-value |

Lipid subset A

| | | | | | |
|---|---|---|---|---|---|
| TG 16:0_32:0 | 0.23 | 0.43 | 1.87 | 0.126 | <0.001 |
| TG 16:0_34:0 | 0.34 | 0.69 | 2.03 | 0.212 | <0.001 |
| TG 16:0_34:3 | 1.60 | 1.07 | 0.67 | 0.367 | 0.005 |
| TG 16:0_36:0 | 0.38 | 0.76 | 2.00 | 0.233 | <0.001 |
| TG 16:0_36:1 | 0.08 | 0.17 | 2.13 | 0.053 | <0.001 |
| TG 16:0_36:3 | 0.87 | 0.56 | 0.64 | 0.235 | 0.014 |
| TG 16:0_36:5 | 0.03 | 0.09 | 3.00 | 0.048 | 0.026 |
| TG 16:0_36:6 | 0.02 | 0.08 | 4.00 | 0.048 | 0.041 |
| TG 16:0_38:0 | 0.12 | 0.37 | 3.08 | 0.160 | 0.001 |
| TG 16:0_38:2 | 0.03 | 0.09 | 3.00 | 0.054 | 0.037 |
| TG 16:0_38:4 | 0.03 | 0.09 | 3.00 | 0.052 | 0.041 |
| TG 16:0_38:8 | 0.02 | 0.08 | 4.00 | 0.052 | 0.041 |
| TG 16:0_40:0 | 0.02 | 0.08 | 4.00 | 0.049 | 0.031 |
| TG 16:1_34:3 | 0.03 | 0.09 | 3.00 | 0.048 | 0.038 |
| TG 16:1_36:0 | 3.14 | 4.36 | 1.39 | 0.780 | 0.001 |
| TG 16:1_36:1 | 0.72 | 0.82 | 1.14 | 0.078 | 0.015 |
| TG 16:1_36:2 | 0.06 | 0.13 | 2.17 | 0.043 | 0.002 |
| TG 16:1_36:3 | 0.02 | 0.08 | 4.00 | 0.049 | 0.033 |
| TG 16:1_36:5 | 0.03 | 0.09 | 3.00 | 0.050 | 0.022 |
| TG 16:1_38:0 | 1.09 | 2.44 | 2.24 | 0.779 | 0.005 |
| TG 16:1_38:1 | 2.27 | 2.84 | 1.25 | 0.487 | 0.036 |
| TG 16:1_38:2 | 0.19 | 0.35 | 1.84 | 0.098 | <0.001 |
| TG 16:1_38:3 | 0.04 | 0.12 | 3.00 | 0.056 | 0.003 |
| TG 16:1_38:4 | 0.03 | 0.08 | 2.67 | 0.050 | 0.031 |
| TG 16:1_38:5 | 0.02 | 0.09 | 4.50 | 0.051 | 0.031 |
| TG 16:1_38:6 | 0.04 | 0.14 | 3.50 | 0.064 | 0.002 |
| TG 16:1_38:7 | 0.23 | 0.51 | 2.22 | 0.170 | 0.001 |
| TG 16:1_40:0 | 0.03 | 0.10 | 3.33 | 0.055 | 0.013 |
| TG 16:1_40:1 | 0.04 | 0.11 | 2.75 | 0.055 | 0.034 |
| TG 16:1_40:2 | 0.03 | 0.09 | 3.00 | 0.048 | 0.021 |
| TG 16:1_40:4 | 0.03 | 0.09 | 3.00 | 0.050 | 0.035 |
| TG 16:1_40:5 | 0.02 | 0.09 | 4.50 | 0.050 | 0.029 |
| TG 16:1_40:7 | 0.03 | 0.09 | 3.00 | 0.053 | 0.028 |
| TG 16:1_40:8 | 0.04 | 0.12 | 3.00 | 0.059 | 0.006 |
| TG 16:1_42:4 | 0.03 | 0.09 | 3.00 | 0.058 | 0.048 |
| TG 16:1_42:8 | 0.02 | 0.08 | 4.00 | 0.049 | 0.043 |
| TG 18:0_30:0 | 0.13 | 0.20 | 1.54 | 0.050 | 0.012 |
| TG 18:0_30:2 | 0.93 | 0.57 | 0.61 | 0.233 | 0.002 |
| TG 18:0_32:1 | 0.22 | 0.31 | 1.41 | 0.055 | 0.001 |
| TG 18:0_32:3 | 4.22 | 2.50 | 0.59 | 1.004 | 0.009 |
| TG 18:0_34:0 | 0.10 | 0.19 | 1.90 | 0.067 | 0.005 |
| TG 18:0_34:1 | 3.46 | 4.10 | 1.18 | 0.503 | 0.019 |
| TG 18:0_34:2 | 21.07 | 15.75 | 0.75 | 3.216 | <0.001 |
| TG 18:0_34:3 | 2.56 | 1.72 | 0.67 | 0.607 | 0.007 |
| TG 18:0_34:4 | 0.11 | 0.21 | 1.91 | 0.072 | 0.004 |
| TG 18:0_34:5 | 0.03 | 0.08 | 2.67 | 0.047 | 0.031 |
| TG 18:0_36:0 | 0.03 | 0.09 | 3.00 | 0.048 | 0.027 |
| TG 18:0_36:1 | 0.66 | 1.26 | 1.91 | 0.346 | 0.004 |
| TG 18:0_36:3 | 6.38 | 4.34 | 0.68 | 1.710 | 0.030 |
| TG 18:0_36:5 | 0.05 | 0.15 | 3.00 | 0.081 | 0.023 |
| TG 18:0_36:6 | 0.03 | 0.09 | 3.00 | 0.050 | 0.037 |
| TG 18:0_36:7 | 0.04 | 0.10 | 2.50 | 0.053 | 0.021 |
| TG 18:0_36:8 | 0.46 | 0.86 | 1.87 | 0.242 | <0.001 |
| TG 18:0_38:2 | 0.02 | 0.08 | 4.00 | 0.050 | 0.035 |
| TG 18:0_38:6 | 0.03 | 0.09 | 3.00 | 0.050 | 0.029 |
| TG 18:0_38:7 | 0.03 | 0.09 | 3.00 | 0.051 | 0.024 |
| TG 18:1_30:0 | 0.46 | 0.74 | 1.61 | 0.188 | 0.002 |
| TG 18:1_30:2 | 0.91 | 0.56 | 0.62 | 0.242 | 0.005 |
| TG 18:1_32:0 | 0.03 | 0.09 | 3.00 | 0.051 | 0.031 |
| TG 18:1_32:1 | 0.51 | 0.85 | 1.67 | 0.216 | 0.001 |
| TG 18:1_32:3 | 6.86 | 7.02 | 1.02 | 1.280 | 0.003 |
| TG 18:1_34:0 | 0.23 | 0.48 | 2.09 | 0.155 | <0.001 |
| TG 18:1_34:1 | 3.64 | 4.92 | 1.35 | 0.846 | 0.002 |
| TG 18:1_34:2 | 12.75 | 9.61 | 0.75 | 1.903 | <0.001 |
| TG 18:1_34:4 | 0.06 | 0.17 | 2.83 | 0.065 | 0.008 |
| TG 18:1_36:0 | 0.03 | 0.09 | 3.00 | 0.056 | 0.036 |
| TG 18:1_36:1 | 0.05 | 0.14 | 2.80 | 0.062 | 0.004 |
| TG 18:1_36:3 | 0.04 | 0.11 | 2.75 | 0.056 | 0.033 |
| TG 18:1_36:4 | 0.03 | 0.10 | 3.33 | 0.052 | 0.018 |
| TG 18:1_36:5 | 0.03 | 0.09 | 3.00 | 0.050 | 0.018 |
| TG 18:1_38:0 | 0.03 | 0.09 | 3.00 | 0.052 | 0.017 |
| TG 18:1_38:1 | 0.06 | 0.13 | 2.17 | 0.060 | 0.026 |
| TG 18:1_38:3 | 0.04 | 0.10 | 2.50 | 0.057 | 0.048 |
| TG 18:1_38:4 | 0.03 | 0.09 | 3.00 | 0.046 | 0.017 |
| TG 18:1_38:5 | 0.03 | 0.09 | 3.00 | 0.054 | 0.028 |
| TG 18:1_38:6 | 0.02 | 0.08 | 4.00 | 0.047 | 0.025 |
| TG 18:1_38:7 | 0.04 | 0.11 | 2.75 | 0.052 | 0.012 |
| TG 18:1_40:8 | 0.03 | 0.09 | 3.00 | 0.053 | 0.045 |
| TG 18:2_30:0 | 0.07 | 0.13 | 1.86 | 0.044 | 0.011 |
| TG 18:2_32:0 | 0.04 | 0.09 | 2.25 | 0.048 | 0.028 |
| TG 18:2_32:1 | 0.14 | 0.25 | 1.79 | 0.063 | <0.001 |
| TG 18:2_34:0 | 0.18 | 0.33 | 1.83 | 0.094 | <0.001 |
| TG 18:2_36:0 | 0.06 | 0.17 | 2.83 | 0.072 | 0.002 |
| TG 18:2_36:1 | 0.17 | 0.33 | 1.94 | 0.097 | <0.001 |
| TG 18:2_36:3 | 0.05 | 0.12 | 2.40 | 0.058 | 0.029 |
| TG 18:2_36:4 | 0.03 | 0.10 | 3.33 | 0.059 | 0.036 |
| TG 18:2_38:1 | 0.02 | 0.08 | 4.00 | 0.050 | 0.032 |
| TG 18:2_38:2 | 0.03 | 0.08 | 2.67 | 0.048 | 0.041 |
| TG 20:0_32:0 | 0.03 | 0.09 | 3.00 | 0.052 | 0.015 |
| TG 20:0_32:1 | 0.03 | 0.09 | 3.00 | 0.048 | 0.020 |
| TG 20:0_34:0 | 0.03 | 0.09 | 3.00 | 0.050 | 0.020 |
| TG 20:0_34:1 | 0.04 | 0.13 | 3.25 | 0.066 | 0.007 |
| TG 20:0_34:7 | 0.02 | 0.08 | 4.00 | 0.047 | 0.031 |
| TG 20:0_34:8 | 0.02 | 0.09 | 4.50 | 0.051 | 0.031 |
| TG 20:0_36:0 | 0.03 | 0.09 | 3.00 | 0.052 | 0.030 |
| TG 20:0_36:1 | 0.03 | 0.10 | 3.33 | 0.057 | 0.026 |
| TG 20:0_36:2 | 0.03 | 0.10 | 3.33 | 0.058 | 0.031 |
| TG 20:0_36:3 | 0.02 | 0.08 | 4.00 | 0.051 | 0.034 |
| TG 20:0_44:2 | 0.03 | 0.09 | 3.00 | 0.049 | 0.035 |
| TG 20:4_30:0 | 0.02 | 0.09 | 4.50 | 0.051 | 0.029 |
| TG 20:4_30:2 | 0.03 | 0.08 | 2.67 | 0.049 | 0.049 |
| TG 20:4_32:0 | 0.02 | 0.08 | 4.00 | 0.046 | 0.028 |
| TG 20:4_32:1 | 0.02 | 0.08 | 4.00 | 0.047 | 0.039 |
| TG 20:4_34:1 | 0.03 | 0.10 | 3.33 | 0.052 | 0.013 |
| TG 20:4_36:0 | 0.03 | 0.08 | 2.67 | 0.051 | 0.046 |
| TG 20:4_36:1 | 0.03 | 0.09 | 3.00 | 0.050 | 0.022 |
| TG 20:4_36:2 | 0.03 | 0.09 | 3.00 | 0.049 | 0.029 |

Lipid subset B

| | | | | | |
|---|---|---|---|---|---|
| Lyso PC(18:1) | 0.11 | 0.16 | 1.45 | 0.039 | 0.022 |
| Lyso PC(18:3) | 0.02 | 0.03 | 1.50 | 0.009 | 0.023 |
| Lyso PC(20:1) | 0.12 | 0.20 | 1.67 | 0.063 | 0.034 |
| PC(30:2) | 0.05 | 0.07 | 1.40 | 0.015 | 0.008 |
| PC(32:0) | 0.34 | 0.39 | 1.15 | 0.036 | 0.008 |
| PC(32:1) | 2.08 | 2.56 | 1.23 | 0.327 | 0.004 |
| PC(32:2) | 0.53 | 0.82 | 1.55 | 0.166 | 0.125 |
| PC(32:4) | 0.04 | 0.08 | 2.00 | 0.018 | 0.033 |
| PC(34:0) | 1.15 | 1.41 | 1.23 | 0.199 | 0.016 |
| PC(34:2) | 11.83 | 7.79 | 0.66 | 2.408 | <0.001 |
| PC(34:3) | 1.69 | 2.03 | 1.20 | 0.243 | 0.006 |
| PC(34:6) | 0.04 | 0.05 | 1.25 | 0.008 | 0.037 |
| PC(36:1) | 1.27 | 1.70 | 1.34 | 0.250 | <0.001 |
| PC(36:7) | 0.08 | 0.13 | 1.63 | 0.030 | 0.004 |
| PC(36:8) | 0.58 | 1.03 | 1.78 | 0.250 | 0.015 |
| PC(38:2) | 0.14 | 0.19 | 1.36 | 0.026 | <0.001 |
| PC(38:7) | 0.05 | 0.06 | 1.20 | 0.007 | 0.017 |
| PC(38:8) | 0.11 | 0.15 | 1.36 | 0.026 | 0.002 |
| PC(38:9) | 0.08 | 0.09 | 1.13 | 0.013 | 0.003 |
| PC(40:6) | 0.05 | 0.07 | 1.40 | 0.014 | 0.023 |
| PC(40:8) | 0.04 | 0.05 | 1.25 | 0.009 | 0.004 |
| PCo(32:0) | 0.11 | 0.19 | 1.73 | 0.044 | 0.012 |
| PCo(32:1) | 0.25 | 0.32 | 1.28 | 0.049 | 0.003 |
| PCo(32:2) | 0.31 | 0.52 | 1.68 | 0.128 | <0.001 |
| PCo(32:3) | 0.10 | 0.13 | 1.30 | 0.021 | 0.005 |
| PCo(34:0) | 0.19 | 0.27 | 1.42 | 0.049 | 0.001 |
| PCo(34:1) | 2.08 | 2.85 | 1.37 | 0.535 | 0.004 |
| PCo(34:3) | 7.11 | 5.32 | 0.75 | 1.094 | <0.001 |
| PCo(34:4) | 1.32 | 1.65 | 1.25 | 0.255 | 0.017 |
| PCo(36:0) | 0.08 | 0.13 | 1.63 | 0.030 | 0.005 |
| PCo(36:1) | 0.58 | 1.03 | 1.78 | 0.250 | 0.015 |
| PCo(36:4) | 2.32 | 1.50 | 0.65 | 0.526 | 0.001 |
| PCo(36:5) | 2.09 | 1.51 | 0.72 | 0.404 | 0.005 |
| PCo(38:0) | 0.05 | 0.06 | 1.20 | 0.007 | 0.017 |
| PCo(38:1) | 0.11 | 0.15 | 1.36 | 0.026 | 0.002 |
| PCo(38:2) | 0.08 | 0.09 | 1.13 | 0.013 | 0.003 |
| PCo(38:5) | 0.88 | 0.61 | 0.69 | 0.199 | 0.008 |
| PCo(40:1) | 0.04 | 0.05 | 1.25 | 0.009 | 0.004 |
| PCp(32:4) | 0.34 | 0.39 | 1.15 | 0.036 | 0.008 |
| PE(36:8) | 0.03 | 0.04 | 1.33 | 0.007 | 0.019 |
| PE(38:5) | 0.19 | 0.26 | 1.37 | 0.051 | 0.003 |
| PE(40:5) | 0.08 | 0.11 | 1.38 | 0.018 | 0.001 |
| PEo(36:1) | 0.03 | 0.04 | 1.33 | 0.007 | 0.019 |
| PEo(36:2) | 0.05 | 0.07 | 1.40 | 0.010 | 0.007 |
| PEo(38:6) | 0.08 | 0.07 | 0.88 | 0.009 | 0.004 |
| SM(d18:0/18:0) | 0.64 | 0.81 | 1.27 | 0.130 | 0.018 |
| SM(d18:0/22:0) | 0.51 | 0.68 | 1.33 | 0.102 | 0.009 |
| SM(d18:0/24:0) | 0.06 | 0.07 | 1.17 | 0.012 | 0.029 |
| SM(d18:1/20:0) | 4.70 | 3.16 | 0.67 | 0.903 | 0.009 |
| SM(d18:1/24:0) | 0.29 | 0.40 | 1.38 | 0.064 | <0.001 |
| SM(d18:1/24:1)15Z)) | 0.29 | 0.33 | 1.14 | 0.036 | 0.028 |
| SM(d18:2/18:1) | 0.03 | 0.05 | 1.67 | 0.010 | 0.014 |
| SM(d18:2/24:1) | 0.21 | 0.18 | 0.86 | 0.028 | 0.035 |

Lipid subset C

| | | | | | |
|---|---|---|---|---|---|
| (9Z)-3- | 0.37 | 0.42 | 1.14 | 0.045 | 0.030 |
| hydroxyoctadecenoyl | | | | | |
| carnitine | | | | | |
| 18:2 Cholesteryl | 0.34 | 0.41 | 1.21 | 0.057 | 0.018 |
| ester | | | | | |
| Cer(d18:1/22:0) | 0.45 | 0.39 | 0.87 | 0.054 | 0.042 |
| DG 16:0_18:1 | 0.83 | 0.50 | 0.60 | 0.185 | 0.010 |
| DG 18:0_18:1 | 0.80 | 0.53 | 0.66 | 0.153 | 0.050 |
| DG 18:1_16:0 | 0.48 | 0.43 | 0.90 | 0.050 | 0.001 |
| DG 18:1_18:1 | 0.73 | 0.51 | 0.70 | 0.140 | 0.006 |
| DG 18:2_16:0 | 0.99 | 0.69 | 0.70 | 0.214 | 0.007 |
| Hexadecanedioic | 0.64 | 0.38 | 0.59 | 0.153 | 0.044 |
| acid mono-L- | | | | | |
| carnitine ester | | | | | |
| PG(34:1) | 0.36 | 0.32 | 0.89 | 0.034 | 0.019 |
| PG(36:2) | 0.91 | 0.61 | 0.67 | 0.239 | <0.001 |
| Stearoylcarnitine | 0.64 | 0.38 | 0.59 | 0.153 | 0.044 |

Lipid subsets A = triglyceride (TG); B = phosphatidylcholine (PC), phosphatidylethanolamine (PE) and sphingomyelin (SM); and C = acyl-carnitine, ceramides (CER), diglyceride (DG), free fatty acids and phosphatidylglycerol (PG), phosphatidylinositol (PI) and phosphatidylserine (PS).
^{a} P-L/F-H.

TABLE 4
F-L and P-H Animals

| | | | | | |
|---|---|---|---|---|---|
| | | | Fold | | P- |
| Lipid, % | F-L | P-H | ratio^{a} | SEM | value |

Lipid subset A

| | | | | | |
|---|---|---|---|---|---|
| TG 16:0_38:0 | 0.19 | 0.27 | 1.42 | 0.064 | 0.030 |
| TG 16:1_38:0 | 1.59 | 2.38 | 1.50 | 0.535 | 0.003 |
| TG 16:1_38:1 | 2.54 | 3.05 | 1.20 | 0.399 | 0.019 |
| TG 16:1_38:7 | 0.27 | 0.41 | 1.52 | 0.104 | 0.007 |
| TG 18:0_36:1 | 0.89 | 1.24 | 1.39 | 0.236 | 0.003 |
| TG 18:0_36:2 | 4.75 | 5.65 | 1.19 | 0.767 | 0.035 |
| TG 18:0_36:8 | 0.51 | 0.71 | 1.39 | 0.156 | 0.016 |
| TG 18:1_32:3 | 2.44 | 1.98 | 0.81 | 0.419 | 0.048 |
| TG 18:1_34:2 | 11.96 | 10.77 | 0.90 | 1.068 | 0.047 |

Lipid subset B

| | | | | | |
|---|---|---|---|---|---|
| Lyso PC(18:1) | 0.11 | 0.14 | 1.27 | 0.024 | 0.015 |
| Lyso PC(18:3) | 0.02 | 0.03 | 1.50 | 0.004 | 0.035 |
| Lyso PC(22:4) | 0.09 | 0.06 | 0.67 | 0.029 | 0.025 |
| PC(30:1) | 0.30 | 0.34 | 1.13 | 0.029 | 0.027 |
| PC(30:2) | 0.05 | 0.06 | 1.20 | 0.011 | 0.004 |
| PC(32:0) | 0.29 | 0.35 | 1.21 | 0.046 | 0.007 |
| PC(32:1) | 1.77 | 2.25 | 1.27 | 0.348 | 0.006 |
| PC(32:2) | 0.46 | 0.70 | 1.52 | 0.152 | 0.001 |
| PC(32:4) | 0.04 | 0.06 | 1.50 | 0.012 | 0.004 |
| PC(34:0) | 0.99 | 1.20 | 1.21 | 0.188 | 0.045 |
| PC(34:2) | 13.96 | 10.44 | 0.75 | 2.625 | 0.011 |
| PC(34:3) | 1.77 | 2.21 | 1.25 | 0.290 | 0.002 |
| PC(36:1) | 1.08 | 1.61 | 1.49 | 0.381 | 0.007 |
| PC(36:2) | 2.24 | 2.70 | 1.21 | 0.371 | 0.022 |
| PC(36:4) | 1.42 | 1.22 | 0.86 | 0.127 | 0.002 |
| PC(36:7) | 0.07 | 0.10 | 1.43 | 0.016 | 0.001 |
| PC(36:8) | 0.52 | 0.80 | 1.54 | 0.197 | 0.005 |
| PC(38:0) | 0.13 | 0.10 | 0.77 | 0.022 | 0.035 |
| PC(38:5) | 0.39 | 0.49 | 1.26 | 0.071 | 0.004 |
| PC(38:6) | 0.16 | 0.20 | 1.25 | 0.027 | 0.021 |
| PC(38:7) | 0.04 | 0.05 | 1.25 | 0.011 | 0.022 |
| PC(38:8) | 0.10 | 0.13 | 1.30 | 0.016 | 0.009 |
| PC(40:10)) | 0.03 | 0.02 | 0.67 | 0.003 | 0.020 |
| PC(40:5) | 0.05 | 0.07 | 1.40 | 0.010 | 0.003 |
| PC(40:6) | 0.05 | 0.06 | 1.20 | 0.010 | 0.015 |
| PC(40:7)) | 0.03 | 0.05 | 1.67 | 0.010 | 0.005 |
| PC(40:8) | 0.03 | 0.04 | 1.33 | 0.005 | 0.022 |
| PCo(32:0) | 0.11 | 0.16 | 1.45 | 0.032 | 0.001 |
| PCo(32:1) | 0.24 | 0.30 | 1.25 | 0.037 | 0.003 |
| PCo(32:2) | 0.24 | 0.43 | 1.79 | 0.126 | 0.001 |
| PCo(34:0) | 0.16 | 0.22 | 1.38 | 0.044 | 0.017 |
| PCo(34:1) | 1.47 | 2.30 | 1.56 | 0.591 | 0.006 |
| PCo(34:2) | 5.48 | 6.68 | 1.22 | 1.047 | 0.039 |
| PCo(34:3) | 8.66 | 6.84 | 0.79 | 1.413 | 0.017 |
| PCo(34:4) | 1.09 | 1.43 | 1.31 | 0.235 | 0.005 |
| PCo(36:0) | 0.07 | 0.10 | 1.43 | 0.016 | 0.001 |
| PCo(36:1) | 0.52 | 0.80 | 1.54 | 0.197 | 0.005 |
| PCo(36:3) | 2.38 | 1.90 | 0.80 | 0.287 | <0.001 |
| PCo(36:4) | 2.82 | 1.91 | 0.68 | 0.586 | 0.001 |
| PCo(36:5) | 3.01 | 1.80 | 0.60 | 0.960 | 0.020 |
| PCo(38:0) | 0.04 | 0.05 | 1.25 | 0.011 | 0.022 |
| PCo(38:1) | 0.10 | 0.13 | 1.30 | 0.016 | 0.009 |
| PCo(38:4) | 0.51 | 0.35 | 0.69 | 0.096 | <0.001 |
| PCo(38:5) | 1.12 | 0.70 | 0.63 | 0.268 | 0.001 |
| PCo(40:0 | 0.03 | 0.05 | 1.67 | 0.010 | 0.005 |
| PCo(40:1) | 0.03 | 0.04 | 1.33 | 0.005 | 0.022 |
| PCo(40:3 | 0.03 | 0.02 | 0.67 | 0.003 | 0.020 |
| PCo(40:4) | 0.05 | 0.04 | 0.80 | 0.007 | 0.029 |
| PCo(40:5) | 0.11 | 0.09 | 0.82 | 0.009 | 0.001 |
| PCp(32:4) | 0.29 | 0.35 | 1.21 | 0.046 | 0.007 |
| PCp(40:6) | 0.13 | 0.10 | 0.77 | 0.022 | 0.035 |
| PE(34:1) | 0.03 | 0.05 | 1.67 | 0.009 | 0.001 |
| PE(36:1) | 0.12 | 0.17 | 1.42 | 0.029 | 0.001 |
| PE(36:3) | 0.14 | 0.19 | 1.36 | 0.028 | 0.004 |
| PE(38:3) | 0.15 | 0.20 | 1.33 | 0.037 | 0.012 |
| PE(38:4) | 0.76 | 0.91 | 1.20 | 0.136 | 0.043 |
| PE(38:5) | 0.14 | 0.24 | 1.71 | 0.057 | <0.001 |
| PE(40:5) | 0.06 | 0.11 | 1.83 | 0.029 | 0.001 |
| PEo(36:5) | 0.05 | 0.04 | 0.80 | 0.009 | 0.045 |
| PEo(38:5) | 0.13 | 0.11 | 0.85 | 0.022 | 0.027 |
| SM(d16:1/18:1) | 0.06 | 0.07 | 1.17 | 0.010 | 0.002 |
| SM(d16:1/22:1) | 0.79 | 0.95 | 1.20 | 0.115 | 0.013 |
| SM(d16:1/24:0) | 1.23 | 1.43 | 1.16 | 0.167 | 0.040 |
| SM(d18:0/18:0) | 0.50 | 0.69 | 1.38 | 0.133 | 0.003 |
| SM(d18:0/22:0) | 0.42 | 0.62 | 1.48 | 0.141 | 0.008 |
| SM(d18:1/18:0) | 2.66 | 2.22 | 0.83 | 0.331 | 0.011 |
| SM(d18:1/20:0) | 5.55 | 4.22 | 0.76 | 0.992 | 0.011 |
| SM(d18:2/18:1) | 0.03 | 0.04 | 1.33 | 0.008 | 0.016 |
| SM(d18:2/22:1) | 0.61 | 0.53 | 0.87 | 0.061 | 0.009 |
| SM(d18:2/24:1) | 0.22 | 0.20 | 0.91 | 0.020 | 0.050 |

Lipid subset C

| | | | | | |
|---|---|---|---|---|---|
| (4Z)-decenoylcarnitine | 0.39 | 0.32 | 0.82 | 0.062 | 0.040 |
| (5Z)-13-carboxytridec- | 0.43 | 0.33 | 0.77 | 0.089 | 0.046 |
| 5-enoylcarnitine | | | | | |
| (9Z,12Z,15Z)-3-hydroxy | 0.43 | 0.34 | 0.79 | 0.069 | 0.019 |
| octadecatrienoylcarnitine | | | | | |
| 3- | 0.42 | 0.35 | 0.83 | 0.059 | 0.049 |
| hydroxyeicosanoylcarnitine | | | | | |
| 9-decenoylcarnitine | 0.39 | 0.32 | 0.82 | 0.062 | 0.040 |
| C16:0 | 0.80 | 1.47 | 1.84 | 0.588 | 0.035 |
| C18:0 | 0.92 | 1.73 | 1.88 | 0.728 | 0.006 |
| Cervonyl carnitine | 0.42 | 0.35 | 0.83 | 0.059 | 0.049 |
| DG 16:0_18:2 | 0.87 | 0.72 | 0.83 | 0.126 | 0.043 |
| DG 18:2_16:0 | 0.91 | 0.69 | 0.76 | 0.155 | 0.019 |
| DG 18:2_18:0 | 0.38 | 0.32 | 0.84 | 0.055 | 0.033 |
| Elaidic carnitine | 0.37 | 0.30 | 0.81 | 0.055 | 0.043 |
| O-oleoylcarnitine | 0.37 | 0.30 | 0.81 | 0.055 | 0.043 |
| Palmitoylcarnitine | 0.43 | 0.33 | 0.77 | 0.089 | 0.046 |
| PI(36:1) | 0.57 | 0.80 | 1.40 | 0.204 | 0.040 |
| PI(38:7) | 0.33 | 0.27 | 0.82 | 0.053 | 0.026 |
| PI(40:0) | 0.93 | 0.61 | 0.66 | 0.286 | 0.048 |
| PIp(42:6) | 0.93 | 0.61 | 0.66 | 0.286 | 0.048 |

Lipid subsets A = triglyceride (TG); B = phosphatidylcholine (PC), phosphatidylethanolamine (PE) and sphingomyelin (SM); and C = acyl-carnitine, ceramides (CER), diglyceride (DG), free fatty acids and phosphatidylglycerol (PG), phosphatidylinositol (PI) and phosphatidylserine (PS).
^{a}P-H/F-L.

TABLE 5
F-H and F-L Animals

| | | | | | |
|---|---|---|---|---|---|
| | | | Fold | | |
| Lipid, % | F-H | F-L | ratio^{a} | SEM | P-value |

Lipid subset A

| | | | | | |
|---|---|---|---|---|---|
| TG 16:0_34:0 | 0.34 | 0.49 | 1.44 | 0.133 | 0.041 |
| TG 16:0_36:0 | 0.38 | 0.55 | 1.45 | 0.116 | 0.004 |
| TG 16:0_36:1 | 0.08 | 0.11 | 1.38 | 0.021 | 0.002 |
| TG 16:0_36:5 | 0.03 | 0.04 | 1.33 | 0.013 | 0.028 |
| TG 16:0_36:6 | 0.02 | 0.04 | 2.00 | 0.012 | 0.036 |
| TG 16:0_38:0 | 0.12 | 0.19 | 1.58 | 0.043 | 0.051 |
| TG 16:0_38:2 | 0.03 | 0.04 | 1.33 | 0.012 | 0.032 |
| TG 16:0_38:4 | 0.03 | 0.04 | 1.33 | 0.013 | 0.035 |
| TG 16:0_38:8 | 0.02 | 0.04 | 2.00 | 0.013 | 0.031 |
| TG 16:0_40:0 | 0.02 | 0.04 | 2.00 | 0.012 | 0.020 |
| TG 16:1_36:0 | 3.14 | 3.89 | 1.24 | 0.607 | 0.024 |
| TG 16:1_36:1 | 0.72 | 0.82 | 1.14 | 0.075 | 0.010 |
| TG 16:1_36:2 | 0.06 | 0.08 | 1.33 | 0.015 | 0.041 |
| TG 16:1_36:3 | 0.02 | 0.04 | 2.00 | 0.013 | 0.029 |
| TG 16:1_36:5 | 0.03 | 0.04 | 1.33 | 0.012 | 0.016 |
| TG 16:1_38:0 | 1.09 | 1.59 | 1.46 | 0.343 | 0.003 |
| TG 16:1_38:2 | 0.19 | 0.28 | 1.47 | 0.070 | 0.017 |
| TG 16:1_38:3 | 0.04 | 0.06 | 1.50 | 0.017 | 0.004 |
| TG 16:1_38:4 | 0.03 | 0.04 | 1.33 | 0.014 | 0.035 |
| TG 16:1_38:5 | 0.02 | 0.04 | 2.00 | 0.013 | 0.028 |
| TG 16:1_38:6 | 0.04 | 0.07 | 1.75 | 0.019 | 0.020 |
| TG 16:1_40:0 | 0.03 | 0.05 | 1.67 | 0.016 | 0.022 |
| TG 16:1_40:2 | 0.03 | 0.05 | 1.67 | 0.013 | 0.017 |
| TG 16:1_40:4 | 0.03 | 0.04 | 1.33 | 0.014 | 0.039 |
| TG 16:1_40:5 | 0.02 | 0.04 | 2.00 | 0.012 | 0.016 |
| TG 16:1_40:7 | 0.03 | 0.04 | 1.33 | 0.014 | 0.037 |
| TG 16:1_40:8 | 0.04 | 0.06 | 1.50 | 0.017 | 0.011 |
| TG 16:1_42:4 | 0.03 | 0.04 | 1.33 | 0.013 | 0.021 |
| TG 16:1_42:8 | 0.02 | 0.04 | 2.00 | 0.013 | 0.030 |
| TG 18:0_30:2 | 0.93 | 0.65 | 0.70 | 0.220 | 0.020 |
| TG 18:0_32:1 | 0.22 | 0.27 | 1.23 | 0.044 | 0.047 |
| TG 18:0_32:3 | 4.22 | 3.08 | 0.73 | 0.753 | 0.002 |
| TG 18:0_34:1 | 3.46 | 4.01 | 1.16 | 0.490 | 0.045 |
| TG 18:0_36:0 | 0.03 | 0.04 | 1.33 | 0.012 | 0.025 |
| TG 18:0_36:1 | 0.66 | 0.89 | 1.35 | 0.172 | 0.011 |
| TG 18:0_36:6 | 0.03 | 0.04 | 1.33 | 0.014 | 0.048 |
| TG 18:0_36:7 | 0.04 | 0.05 | 1.25 | 0.014 | 0.009 |
| TG 18:0_38:2 | 0.02 | 0.04 | 2.00 | 0.013 | 0.030 |
| TG 18:0_38:6 | 0.03 | 0.04 | 1.33 | 0.013 | 0.014 |
| TG 18:0_38:7 | 0.03 | 0.04 | 1.33 | 0.013 | 0.031 |
| TG 18:1_32:0 | 0.03 | 0.04 | 1.33 | 0.012 | 0.025 |
| TG 18:1_34:0 | 0.23 | 0.35 | 1.52 | 0.073 | <0.001 |
| TG 18:1_34:1 | 3.64 | 4.55 | 1.25 | 0.728 | 0.021 |
| TG 18:1_34:4 | 0.06 | 0.10 | 1.67 | 0.027 | 0.019 |
| TG 18:1_36:0 | 0.03 | 0.04 | 1.33 | 0.012 | 0.019 |
| TG 18:1_36:1 | 0.05 | 0.07 | 1.40 | 0.018 | 0.009 |
| TG 18:1_36:3 | 0.04 | 0.07 | 1.75 | 0.019 | 0.007 |
| TG 18:1_36:4 | 0.03 | 0.06 | 2.00 | 0.018 | 0.005 |
| TG 18:1_36:5 | 0.03 | 0.05 | 1.67 | 0.015 | 0.029 |
| TG 18:1_38:0 | 0.03 | 0.05 | 1.67 | 0.014 | 0.004 |
| TG 18:1_38:4 | 0.03 | 0.05 | 1.67 | 0.015 | 0.044 |
| TG 18:1_38:5 | 0.03 | 0.04 | 1.33 | 0.014 | 0.040 |
| TG 18:1_38:6 | 0.02 | 0.04 | 2.00 | 0.013 | 0.026 |
| TG 18:1_40:8 | 0.03 | 0.04 | 1.33 | 0.014 | 0.038 |
| TG 18:2_32:0 | 0.04 | 0.06 | 1.50 | 0.017 | 0.020 |
| TG 18:2_34:0 | 0.18 | 0.27 | 1.50 | 0.082 | 0.034 |
| TG 18:2_36:0 | 0.06 | 0.10 | 1.67 | 0.031 | 0.006 |
| TG 18:2_36:1 | 0.17 | 0.25 | 1.47 | 0.062 | 0.018 |
| TG 18:2_38:1 | 0.02 | 0.04 | 2.00 | 0.013 | 0.022 |
| TG 18:2_38:2 | 0.03 | 0.04 | 1.33 | 0.013 | 0.030 |
| TG 20:0_32:0 | 0.03 | 0.04 | 1.33 | 0.013 | 0.020 |
| TG 20:0_32:1 | 0.03 | 0.04 | 1.33 | 0.011 | 0.027 |
| TG 20:0_32:2 | 0.03 | 0.04 | 1.33 | 0.012 | 0.028 |
| TG 20:0_34:0 | 0.03 | 0.05 | 1.67 | 0.014 | 0.013 |
| TG 20:0_34:1 | 0.04 | 0.06 | 1.50 | 0.013 | 0.019 |
| TG 20:0_34:7 | 0.02 | 0.04 | 2.00 | 0.012 | 0.023 |
| TG 20:0_34:8 | 0.02 | 0.04 | 2.00 | 0.013 | 0.023 |
| TG 20:0_36:0 | 0.03 | 0.04 | 1.33 | 0.012 | 0.029 |
| TG 20:0_36:1 | 0.03 | 0.05 | 1.67 | 0.015 | 0.019 |
| TG 20:0_36:2 | 0.03 | 0.05 | 1.67 | 0.013 | 0.011 |
| TG 20:0_36:3 | 0.02 | 0.04 | 2.00 | 0.013 | 0.023 |
| TG 20:0_44:2 | 0.03 | 0.04 | 1.33 | 0.013 | 0.037 |
| TG 20:4_30:0 | 0.02 | 0.04 | 2.00 | 0.013 | 0.024 |
| TG 20:4_30:2 | 0.03 | 0.04 | 1.33 | 0.012 | 0.041 |
| TG 20:4_32:0 | 0.02 | 0.04 | 2.00 | 0.013 | 0.029 |
| TG 20:4_32:1 | 0.02 | 0.04 | 2.00 | 0.013 | 0.029 |
| TG 20:4_34:1 | 0.03 | 0.05 | 1.67 | 0.018 | 0.012 |
| TG 20:4_36:0 | 0.03 | 0.04 | 1.33 | 0.013 | 0.026 |
| TG 20:4_36:1 | 0.03 | 0.04 | 1.33 | 0.015 | 0.020 |
| TG 20:4_36:2 | 0.03 | 0.04 | 1.33 | 0.014 | 0.022 |

Lipid subset B

| | | | | | |
|---|---|---|---|---|---|
| PC(32:0) | 0.34 | 0.30 | 0.88 | 0.040 | 0.007 |
| PC(36:7) | 0.08 | 0.08 | 1.00 | 0.008 | 0.026 |
| PC(36:8) | 0.58 | 0.55 | 0.95 | 0.109 | 0.011 |
| PC(40:7) | 0.05 | 0.04 | 0.80 | 0.011 | 0.035 |
| PCo(34:1) | 2.08 | 1.70 | 0.82 | 0.506 | 0.031 |
| PCo(34:3) | 7.11 | 8.16 | 1.15 | 1.186 | 0.028 |
| PCo(36:0) | 0.08 | 0.08 | 1.00 | 0.008 | 0.026 |
| PCo(36:1) | 0.58 | 0.55 | 0.95 | 0.109 | 0.011 |
| PCo(36:3) | 1.99 | 2.26 | 1.14 | 0.347 | 0.015 |
| PCo(36:4) | 2.32 | 2.61 | 1.13 | 0.468 | 0.043 |
| PCo(38:4) | 0.41 | 0.46 | 1.12 | 0.084 | 0.038 |
| PCo(38:5) | 0.88 | 1.00 | 1.14 | 0.222 | 0.050 |
| PCo(40:0) | 0.05 | 0.04 | 0.80 | 0.011 | 0.035 |
| PCp(32:4) | 0.34 | 0.30 | 0.88 | 0.040 | 0.007 |
| PE(34:1) | 0.05 | 0.04 | 0.80 | 0.009 | 0.002 |
| PE(34:2) | 0.04 | 0.04 | 1.00 | 0.006 | 0.016 |
| PE(36:0) | 0.04 | 0.03 | 0.75 | 0.008 | 0.047 |
| PE(36:1) | 0.18 | 0.14 | 0.78 | 0.037 | 0.004 |
| PE(36:8) | 0.03 | 0.03 | 1.00 | 0.006 | 0.011 |
| PE(38:0) | 0.04 | 0.03 | 0.75 | 0.007 | 0.043 |
| PE(38:5) | 0.19 | 0.16 | 0.84 | 0.038 | 0.038 |
| PE(40:4) | 0.05 | 0.05 | 1.00 | 0.008 | 0.012 |
| PE(40:5) | 0.08 | 0.07 | 0.88 | 0.015 | 0.009 |
| PE(40:6) | 0.04 | 0.03 | 0.75 | 0.007 | 0.043 |
| PEo(36:1) | 0.03 | 0.03 | 1.00 | 0.006 | 0.011 |
| PEo(36:2) | 0.05 | 0.05 | 1.00 | 0.008 | 0.038 |
| PEo(38:6) | 0.08 | 0.07 | 0.88 | 0.013 | 0.013 |
| PEo(40:6) | 0.06 | 0.06 | 1.00 | 0.011 | 0.026 |
| PEp(36:6) | 0.04 | 0.03 | 0.75 | 0.008 | 0.047 |
| SM(d18:0/14:0) | 0.03 | 0.02 | 0.67 | 0.006 | 0.038 |
| SM(d18:0/18:0) | 0.64 | 0.52 | 0.81 | 0.130 | 0.046 |
| SM(d18:0/24:0) | 0.06 | 0.07 | 1.17 | 0.008 | 0.036 |
| SM(d18:1/18:0) | 2.31 | 2.47 | 1.07 | 0.291 | 0.028 |
| SM(d18:1/20:0) | 4.70 | 5.19 | 1.10 | 0.763 | 0.048 |
| SM(d18:1/24:0) | 0.29 | 0.30 | 1.03 | 0.032 | 0.015 |
| SM(d18:1/24:1)15Z)) | 0.29 | 0.31 | 1.07 | 0.030 | 0.047 |

Lipid subset C

| | | | | | |
|---|---|---|---|---|---|
| DG 18:2_18:0 | 0.34 | 0.37 | 1.09 | 0.076 | 0.031 |

Lipid subsets A = triglyceride (TG); B = phosphatidylcholine (PC), phosphatidylethanolamine (PE) and sphingomyelin (SM); and C = acyl-carnitine, ceramides (CER), diglyceride (DG), free fatty acids and phosphatidylglycerol (PG), phosphatidylinositol (PI) and phosphatidylserine (PS).
^{a}F-L/F-H.

TABLE 6
P-H and P-L Animals

| | | | | | |
|---|---|---|---|---|---|
| | | | Fold | | P- |
| Lipid, % | P-H | P-L | ratioª | SEM | value |

Lipid subset A

| | | | | | |
|---|---|---|---|---|---|
| TG 16:0_32:1 | 0.25 | 0.31 | 1.24 | 0.046 | 0.016 |
| TG 16:1_34:2 | 0.18 | 0.23 | 1.28 | 0.037 | 0.033 |
| TG 18:0_30:0 | 0.14 | 0.20 | 1.43 | 0.047 | 0.044 |
| TG 18:0_34:4 | 0.14 | 0.21 | 1.50 | 0.059 | 0.039 |
| TG 18:0_36:2 | 5.65 | 5.08 | 0.90 | 0.508 | 0.044 |
| TG 18:0_36:3 | 5.14 | 4.34 | 0.84 | 0.578 | 0.008 |
| TG_18:2_32:1 | 0.20 | 0.25 | 1.25 | 0.038 | 0.039 |

Lipid subset B

| | | | | | |
|---|---|---|---|---|---|
| Lyso PC(16:0) | 0.06 | 0.06 | 1.00 | 0.007 | 0.011 |
| Lyso PC(17:0) | 0.02 | 0.02 | 1.00 | 0.005 | 0.005 |
| Lyso PC(17:1) | 0.02 | 0.02 | 1.00 | 0.005 | 0.016 |
| Lyso PC(20:5) | 0.02 | 0.03 | 1.50 | 0.005 | 0.032 |
| PC(32:0) | 0.35 | 0.39 | 1.11 | 0.036 | 0.044 |
| PCp(32:4) | 0.35 | 0.39 | 1.11 | 0.036 | 0.044 |
| PC(32:1) | 2.25 | 2.56 | 1.14 | 0.253 | 0.026 |
| PC(32:2) | 0.70 | 0.82 | 1.17 | 0.105 | 0.039 |
| PC(32:4) | 0.06 | 0.08 | 1.33 | 0.011 | 0.003 |
| PC(32:5) | 0.03 | 0.04 | 1.33 | 0.008 | 0.016 |
| PC(34:0) | 1.20 | 1.41 | 1.18 | 0.165 | 0.018 |
| PC(34:1) | 14.58 | 16.89 | 1.16 | 1.950 | 0.032 |
| PC(34:2) | 10.44 | 7.79 | 0.75 | 1.897 | 0.007 |
| PC(34:4) | 2.03 | 2.69 | 1.33 | 0.597 | 0.049 |
| PC(34:5) | 0.39 | 0.51 | 1.31 | 0.099 | 0.028 |
| PC(34:6) | 0.04 | 0.05 | 1.25 | 0.007 | 0.005 |
| PC(36:7) | 0.10 | 0.13 | 1.30 | 0.024 | 0.002 |
| PCo(36:0) | 0.10 | 0.13 | 1.30 | 0.024 | 0.002 |
| PC(36:8) | 0.80 | 1.03 | 1.29 | 0.166 | 0.005 |
| PCo(36:1) | 0.80 | 1.03 | 1.29 | 0.166 | 0.005 |
| PC(38:1) | 0.17 | 0.18 | 1.06 | 0.012 | 0.047 |
| PC(38:2) | 0.17 | 0.19 | 1.12 | 0.014 | 0.004 |
| PC(38:3) | 0.16 | 0.14 | 0.88 | 0.016 | 0.018 |
| PC(38:8) | 0.13 | 0.15 | 1.15 | 0.017 | 0.037 |
| PCo(38:1) | 0.13 | 0.15 | 1.15 | 0.017 | 0.037 |
| PC(42:1) | 0.01 | 0.02 | 2.00 | 0.004 | 0.043 |
| PC(42:10) | 0.02 | 0.02 | 1.00 | 0.005 | 0.005 |
| PCo(42:3) | 0.02 | 0.02 | 1.00 | 0.005 | 0.005 |
| PC(42:5) | 0.02 | 0.02 | 1.00 | 0.004 | 0.034 |
| PC(42:8) | 0.02 | 0.02 | 1.00 | 0.005 | 0.049 |
| PCo(42:1) | 0.02 | 0.02 | 1.00 | 0.005 | 0.049 |
| PCo(32:0) | 0.16 | 0.19 | 1.19 | 0.023 | 0.031 |
| PCo(34:0) | 0.22 | 0.27 | 1.23 | 0.037 | 0.014 |
| PCo(34:1) | 2.30 | 2.85 | 1.24 | 0.457 | 0.028 |
| PCo(34:3) | 6.84 | 5.32 | 0.78 | 1.140 | 0.012 |
| PCo(34:4) | 1.43 | 1.65 | 1.15 | 0.185 | 0.032 |
| PCo(36:3) | 1.90 | 1.59 | 0.84 | 0.235 | 0.014 |
| PCo(36:4) | 1.91 | 1.50 | 0.79 | 0.325 | 0.020 |
| PE(18:1) | 0.02 | 0.03 | 1.50 | 0.007 | 0.048 |
| PE(18:3) | 0.01 | 0.02 | 2.00 | 0.005 | 0.030 |
| PE(20:1) | 0.02 | 0.03 | 1.50 | 0.006 | 0.003 |
| PE(20:3) | 0.02 | 0.02 | 1.00 | 0.006 | 0.027 |
| PE(32:1) | 0.01 | 0.02 | 2.00 | 0.004 | 0.046 |
| PE(36:1) | 0.17 | 0.21 | 1.24 | 0.033 | 0.011 |
| PE(36:6) | 0.02 | 0.02 | 1.00 | 0.005 | 0.024 |
| PE(36:8) | 0.03 | 0.04 | 1.33 | 0.008 | 0.007 |
| PEo(36:1) | 0.03 | 0.04 | 1.33 | 0.008 | 0.007 |
| PE(38:9) | 0.02 | 0.02 | 1.00 | 0.005 | 0.035 |
| PEo(38:2) | 0.02 | 0.02 | 1.00 | 0.005 | 0.035 |
| PEo(34:0) | 0.02 | 0.02 | 1.00 | 0.003 | 0.023 |
| PEo(34:1) | 0.02 | 0.03 | 1.50 | 0.006 | 0.043 |
| PEo(40:3) | 0.02 | 0.02 | 1.00 | 0.003 | 0.010 |
| PE(40:10) | 0.02 | 0.02 | 1.00 | 0.003 | 0.010 |
| PEo(40:5) | 0.05 | 0.05 | 1.00 | 0.003 | 0.014 |
| PEp(36:5) | 0.02 | 0.02 | 1.00 | 0.004 | 0.025 |
| SM(d16:1/24:1) | 0.82 | 0.73 | 0.89 | 0.076 | 0.041 |
| SM(d18:0/18:0) | 0.69 | 0.81 | 1.17 | 0.096 | 0.027 |
| SM(d18:0/20:0) | 5.44 | 6.38 | 1.17 | 0.816 | 0.037 |
| SM(d18:1/20:0) | 4.22 | 3.16 | 0.75 | 0.764 | 0.008 |
| SM(d18:1/24:0) | 0.34 | 0.40 | 1.18 | 0.046 | 0.018 |
| SM(d18:1/26:1)17Z)) | 0.02 | 0.03 | 1.50 | 0.005 | 0.010 |
| SM(d18:2/20:1) | 0.53 | 0.67 | 1.26 | 0.130 | 0.046 |
| SM(d18:2/24:1) | 0.20 | 0.18 | 0.90 | 0.012 | 0.004 |

Lipid subset C

| | | | | | |
|---|---|---|---|---|---|
| (9Z,12Z,15Z)-3-hydroxy- | 0.34 | 0.39 | 1.15 | 0.037 | 0.006 |
| octadecatrienoylcarnitine | | | | | |
| 18:2 Cholesteryl ester | 0.35 | 0.39 | 1.11 | 0.036 | 0.015 |
| C18:1 | 0.27 | 0.30 | 1.11 | 0.027 | 0.021 |
| Clupanodonyl carnitine | 0.30 | 0.32 | 1.07 | 0.019 | 0.045 |
| DG 16:0_18:1 | 0.30 | 0.35 | 1.17 | 0.038 | 0.021 |
| Docosa-4,7,10,13,16- | 0.30 | 0.32 | 1.07 | 0.019 | 0.045 |
| pentaenoyl carnitine | | | | | |
| Fumarycarnitine | 0.33 | 0.38 | 1.15 | 0.037 | 0.044 |
| Hexadecanedioic acid mono- | 0.64 | 0.79 | 1.23 | 0.104 | 0.035 |
| L-carnitine ester | | | | | |
| Hexanoylcarnitine | 0.33 | 0.38 | 1.15 | 0.037 | 0.044 |
| O-(11- | 0.69 | 0.50 | 0.72 | 0.150 | 0.041 |
| carboxyundecanoyl)carnitine | | | | | |
| PS(28:0) | 0.67 | 0.53 | 0.79 | 0.108 | 0.008 |
| Stearoylcarnitine | 0.64 | 0.79 | 1.23 | 0.104 | 0.035 |

Lipid subsets A = triglyceride (TG); B = phosphatidylcholine (PC), phosphatidylethanolamine (PE) and sphingomyelin (SM); and C = acyl-carnitine, ceramides (CER), diglyceride (DG), free fatty acids and phosphatidylglycerol (PG), phosphatidylinositol (PI) and phosphatidylserine (PS).
^{a}P-L/P-H.

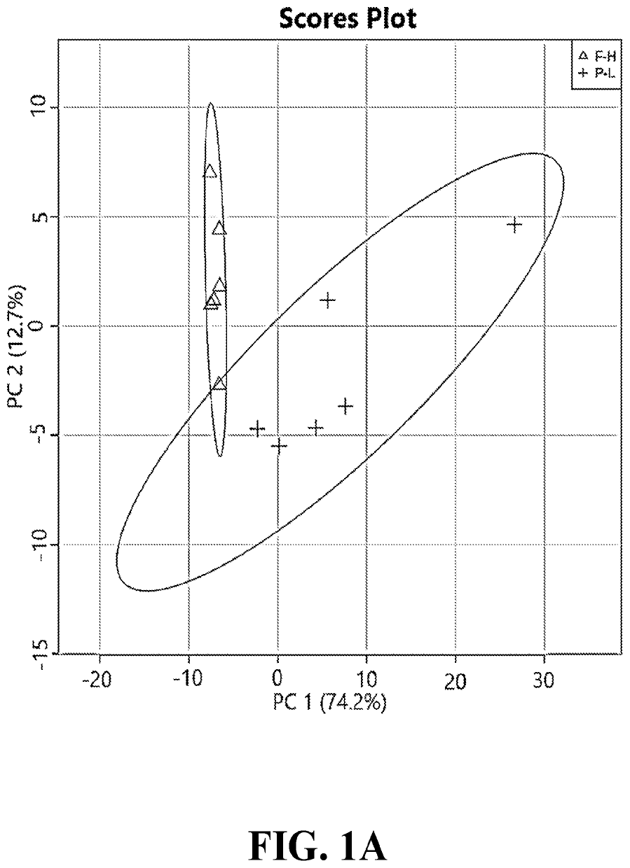

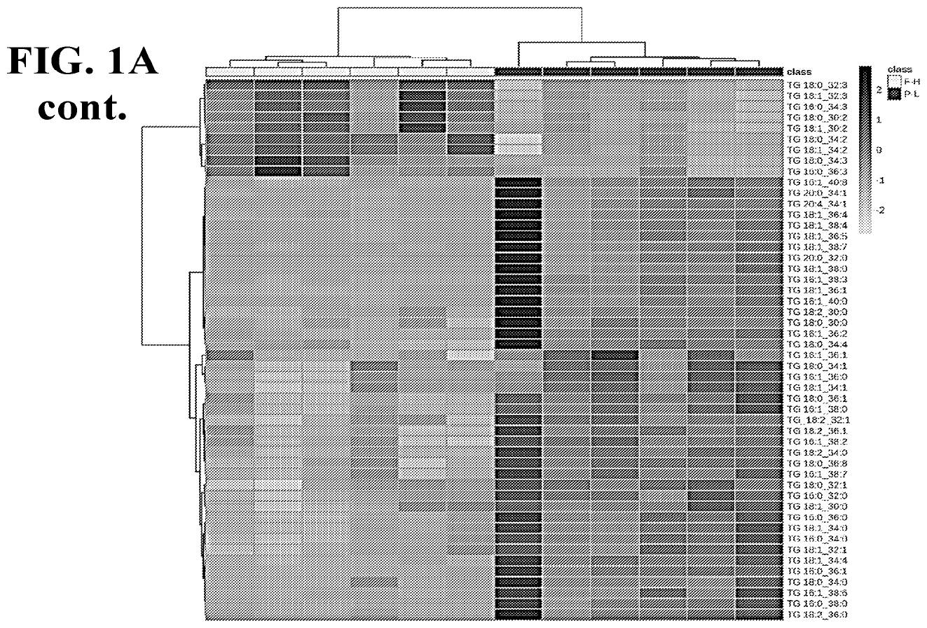

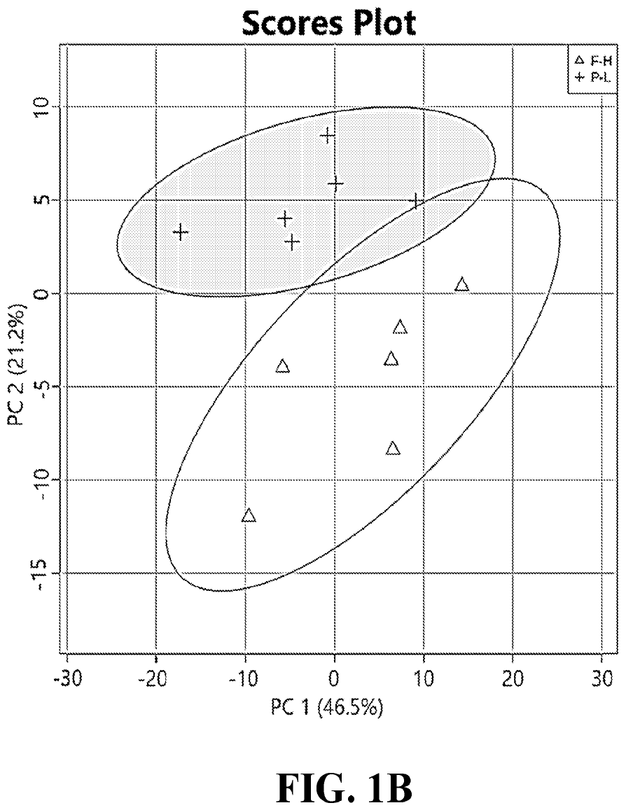

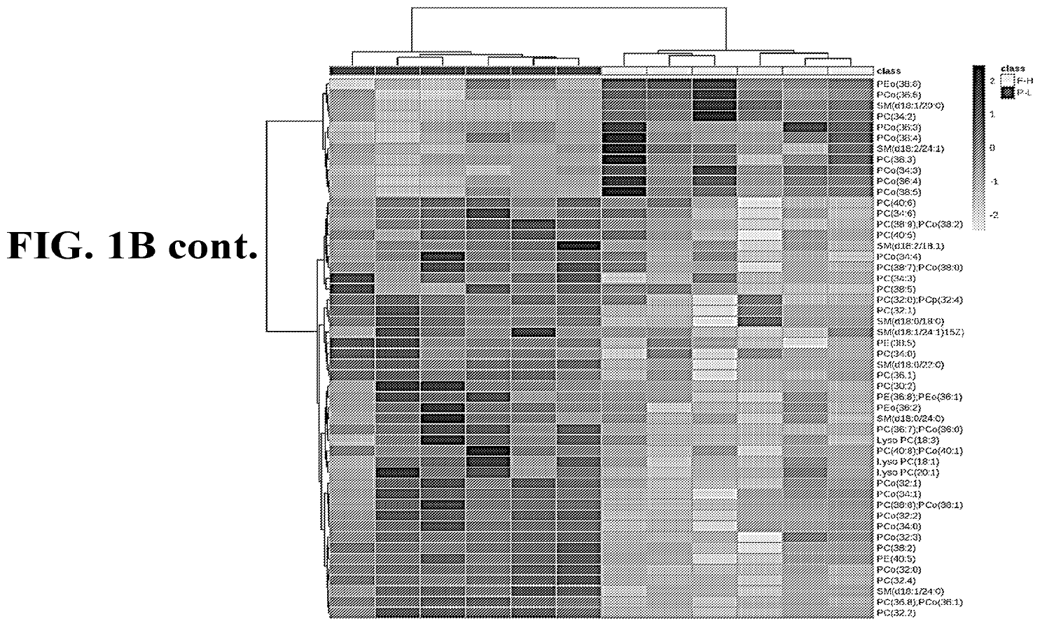

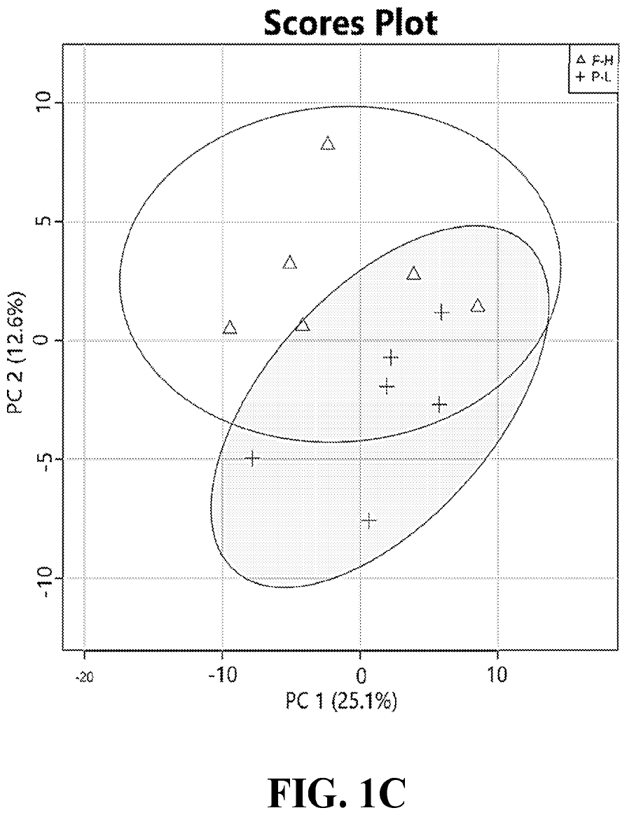

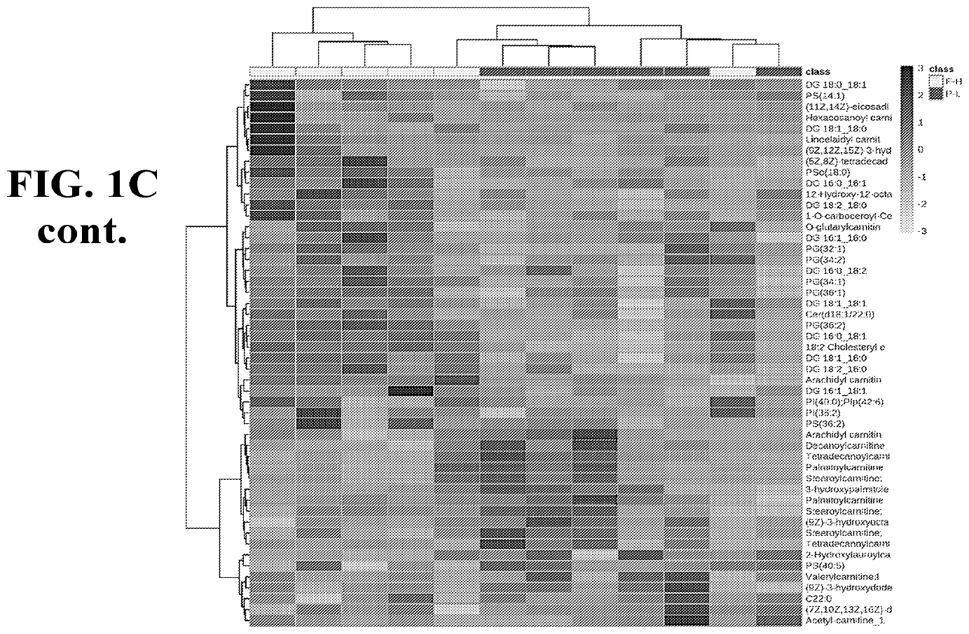

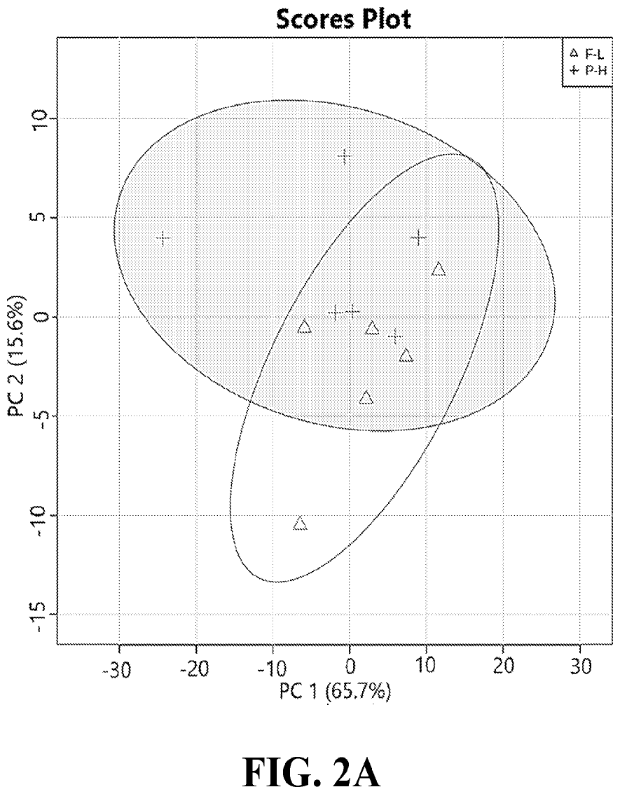

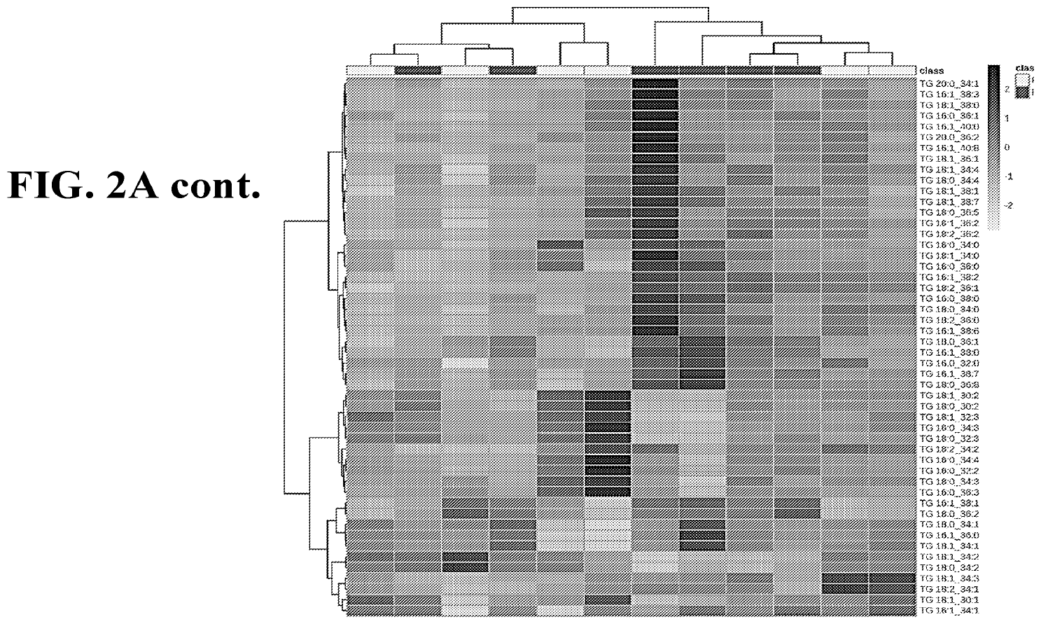

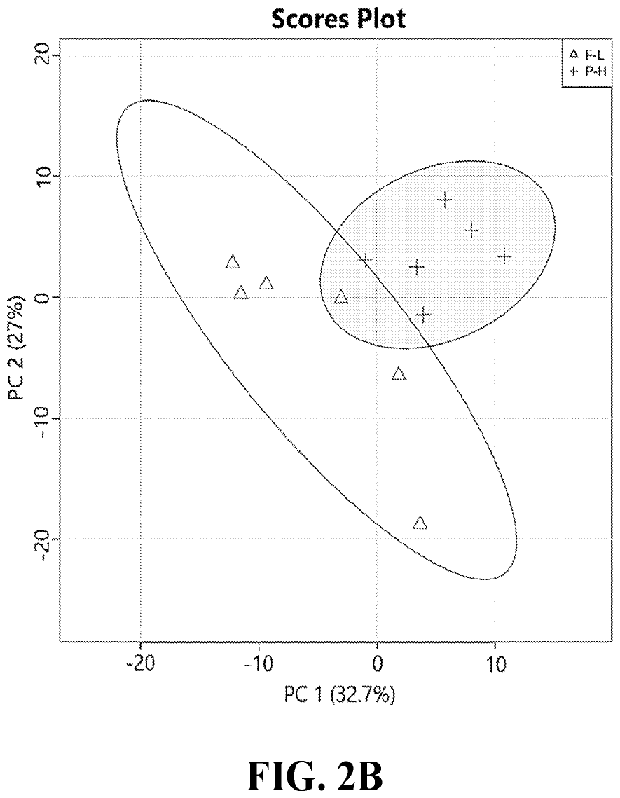

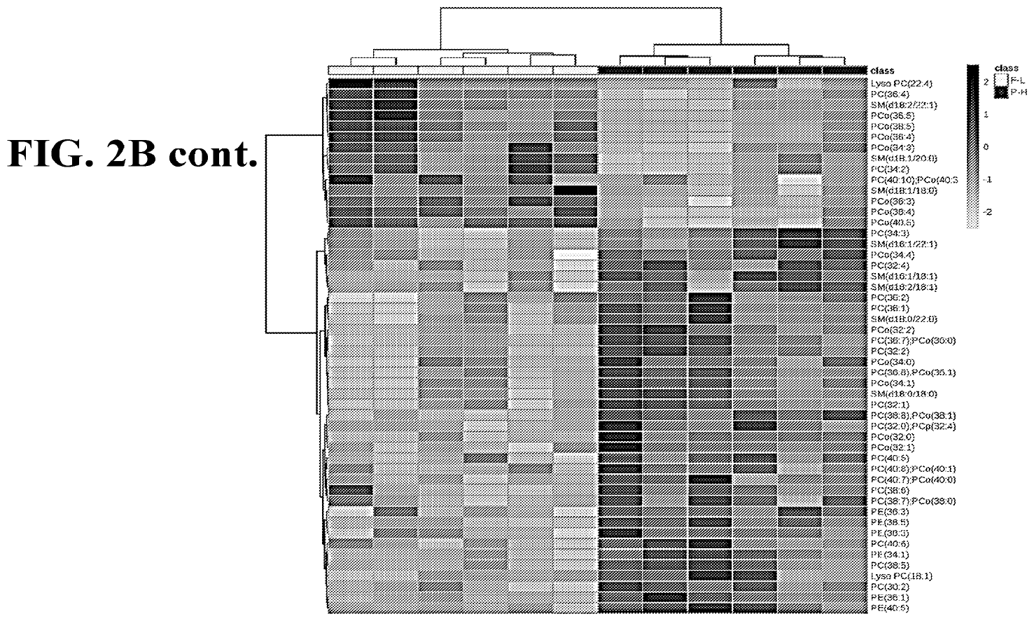

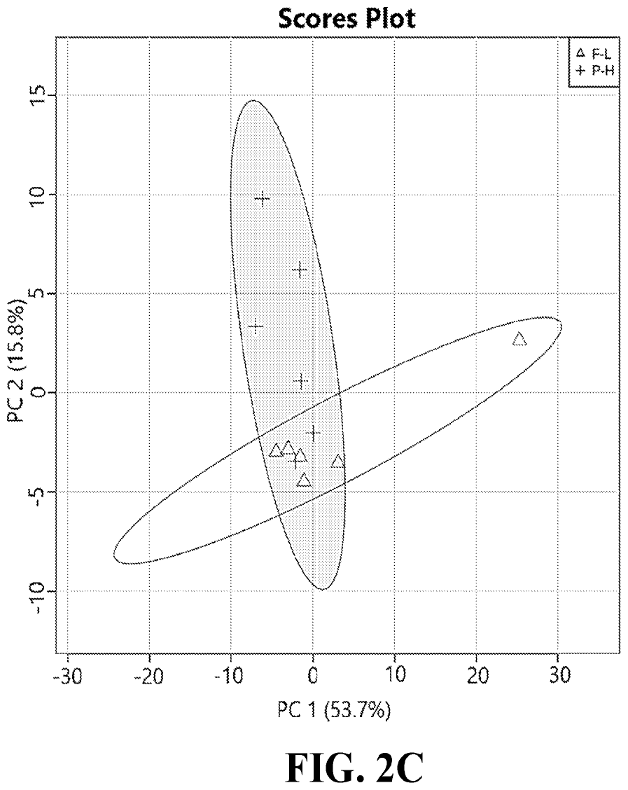

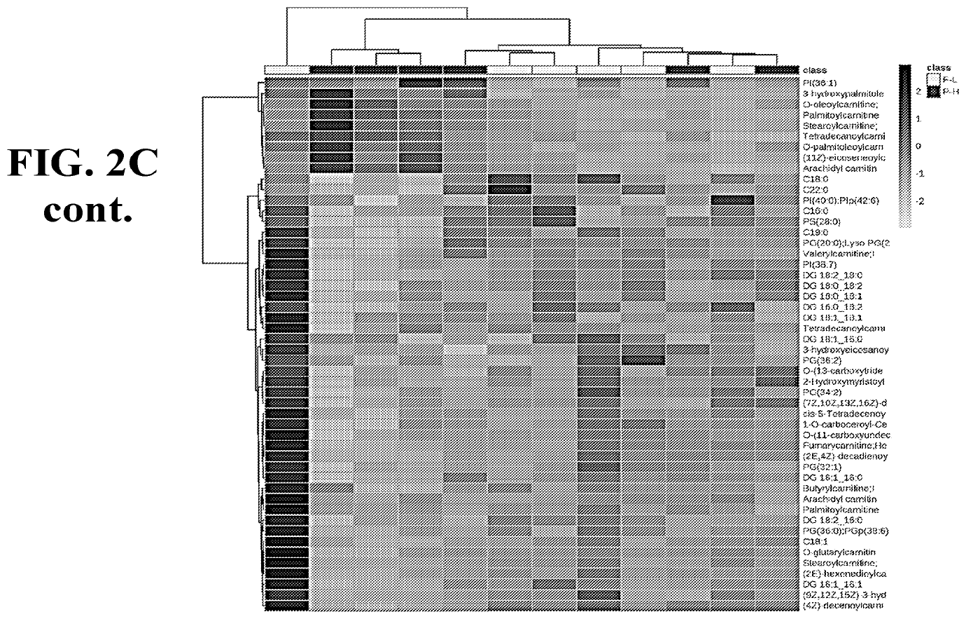

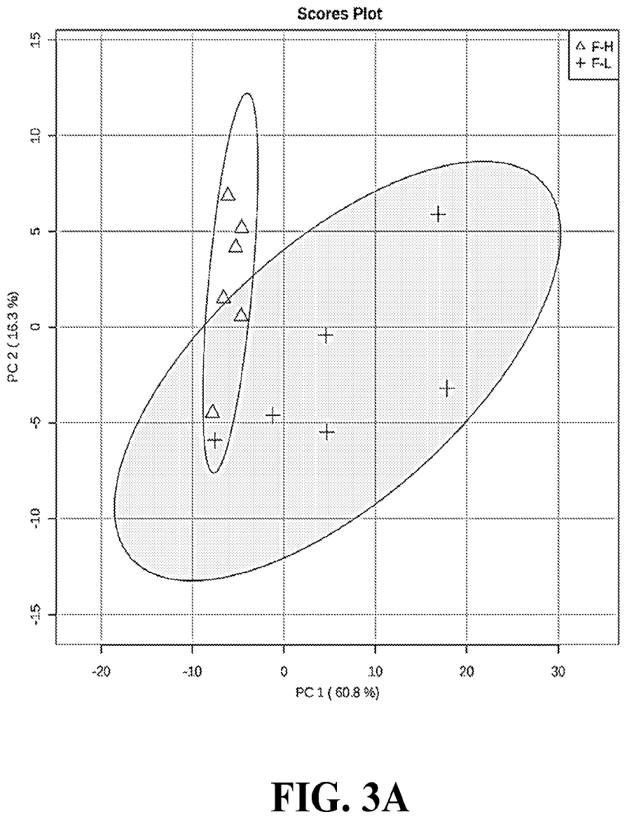

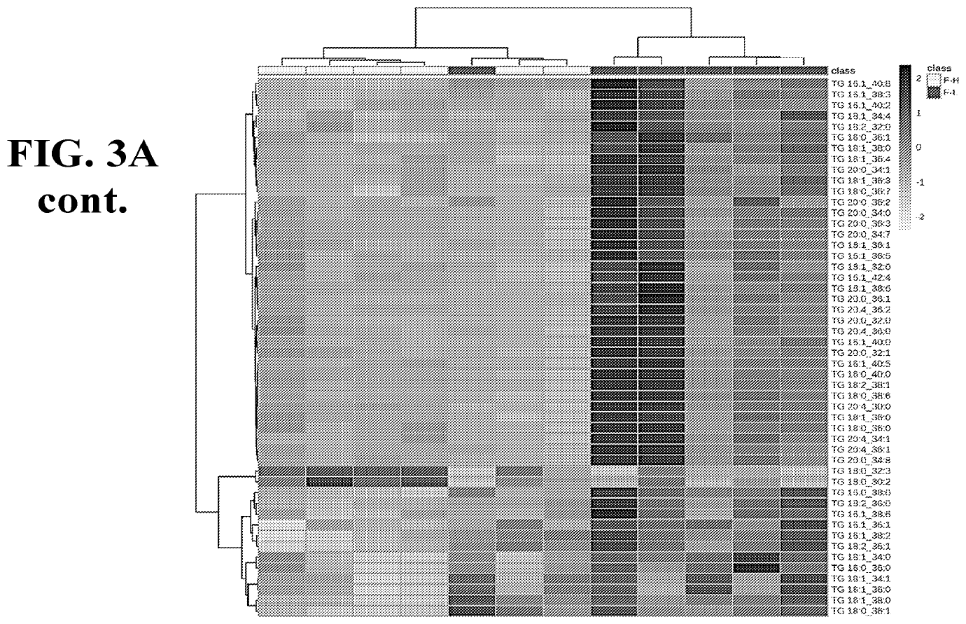

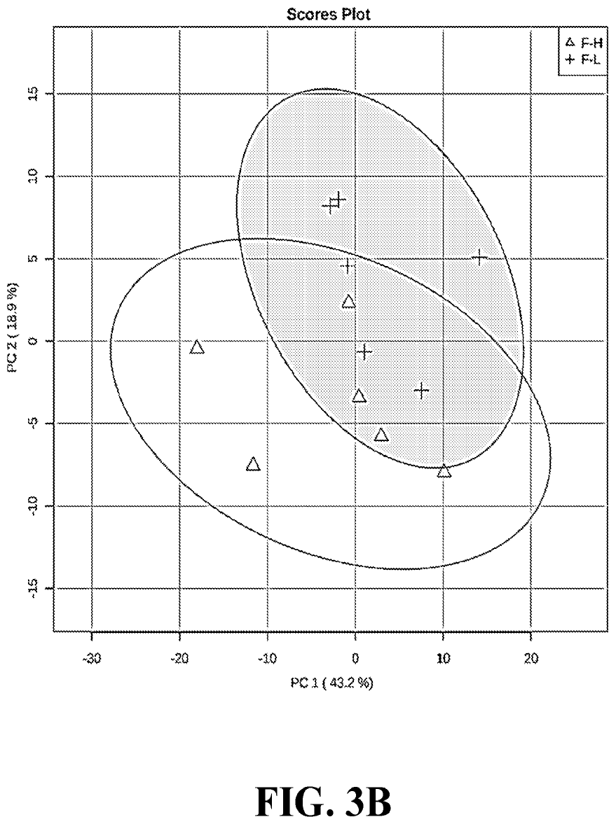

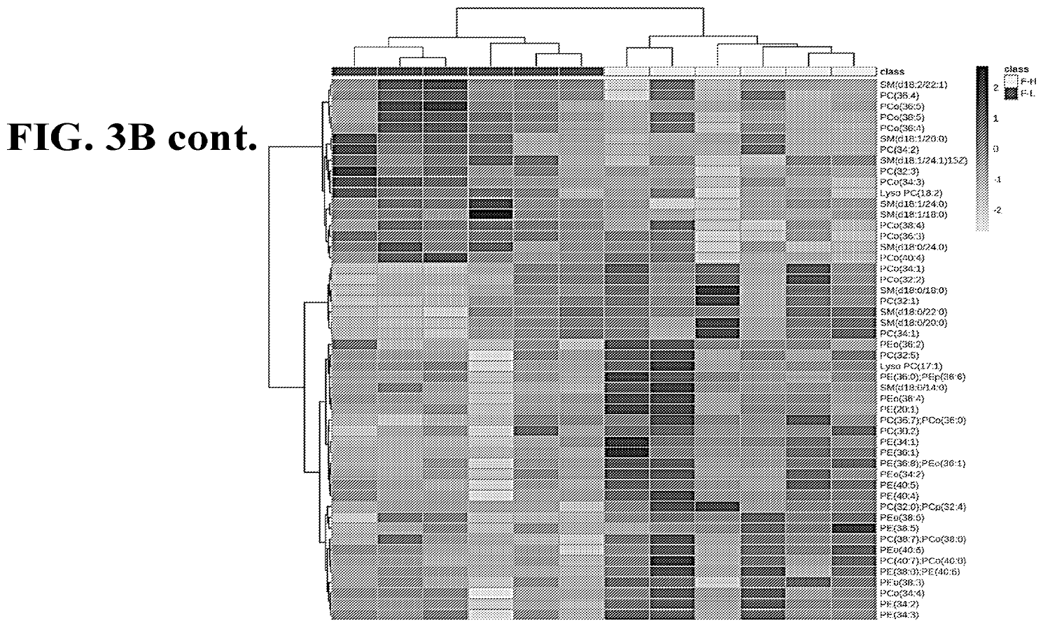

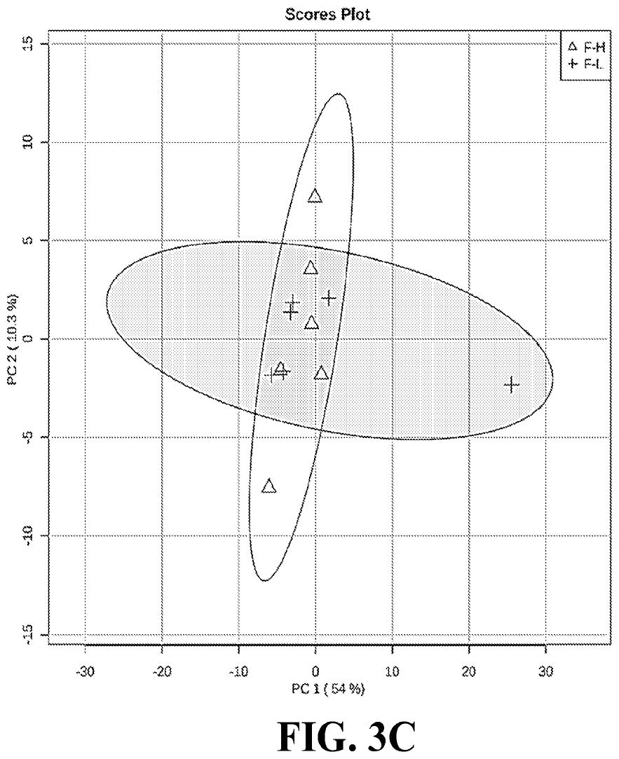

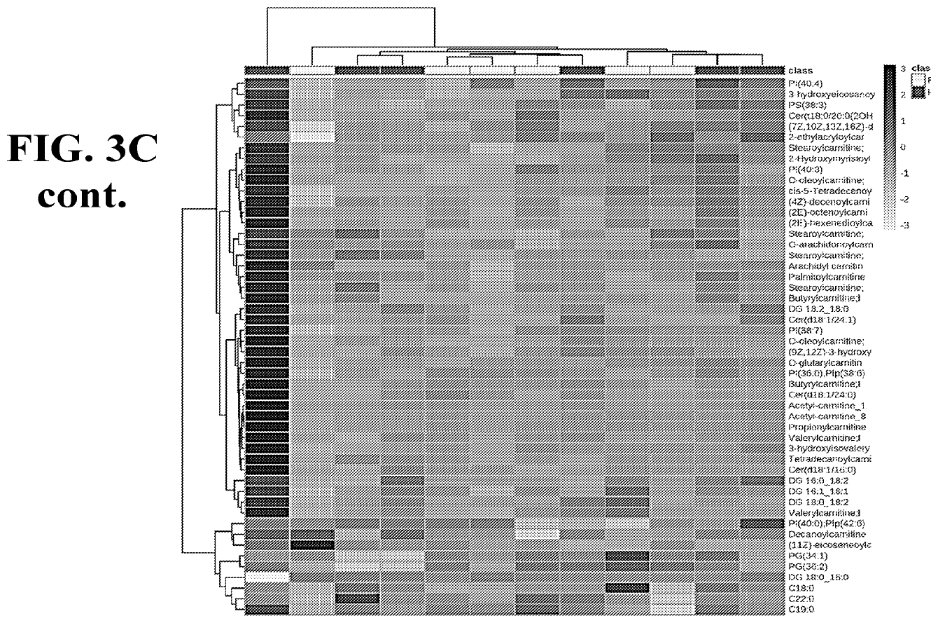

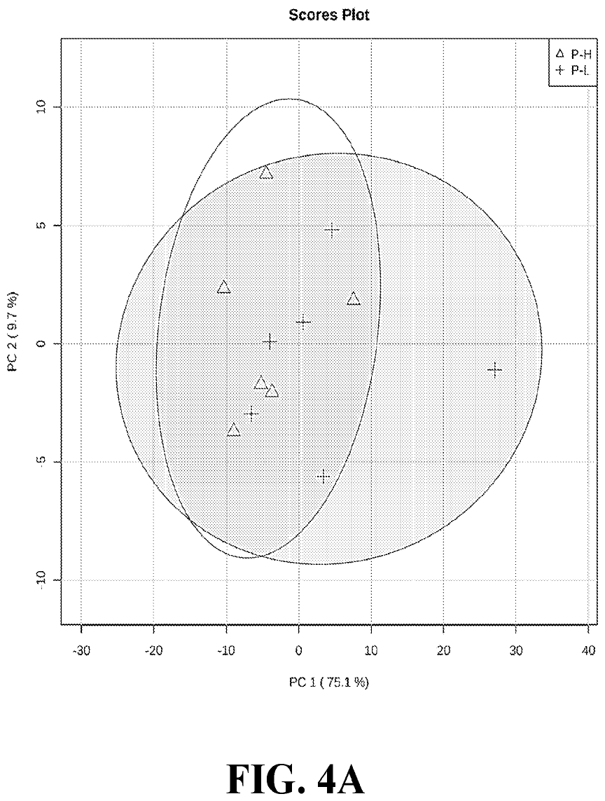

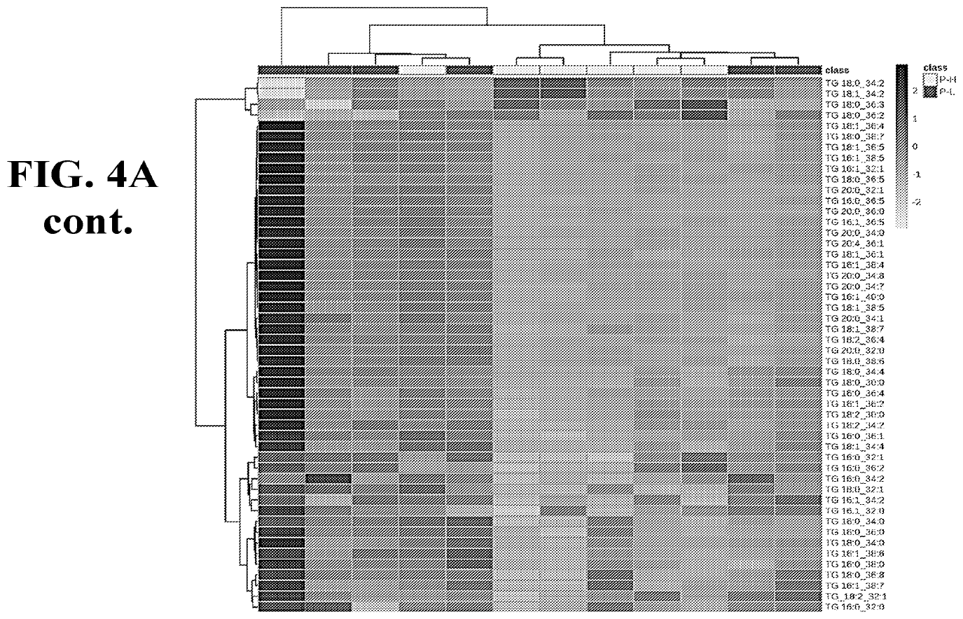

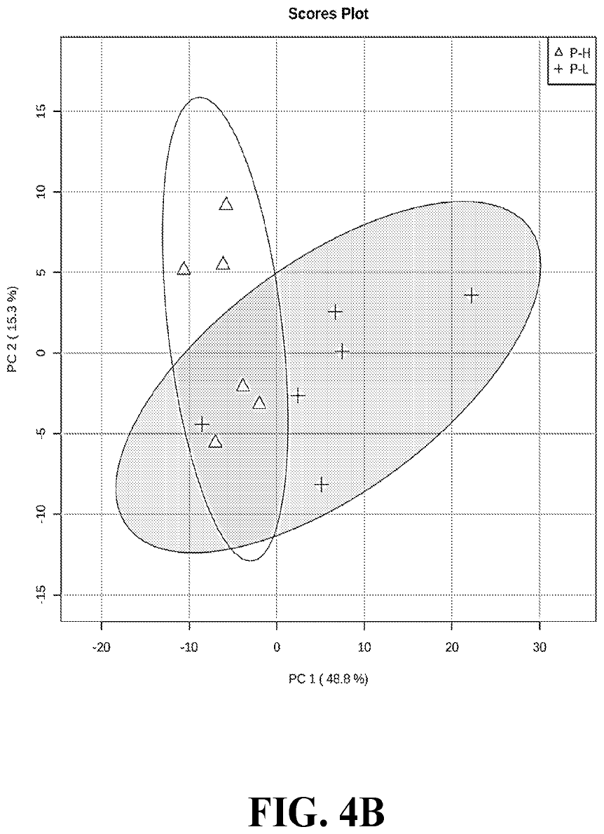

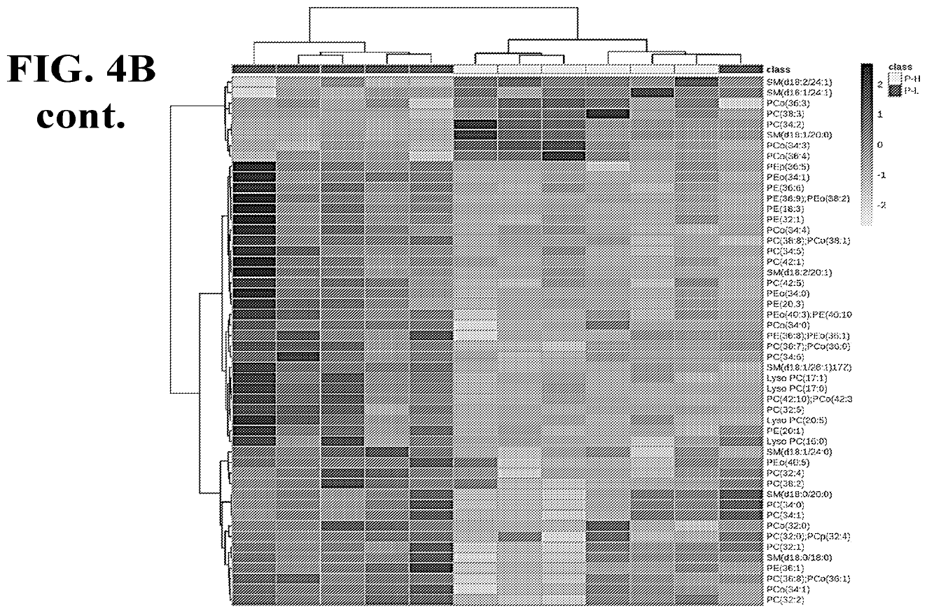

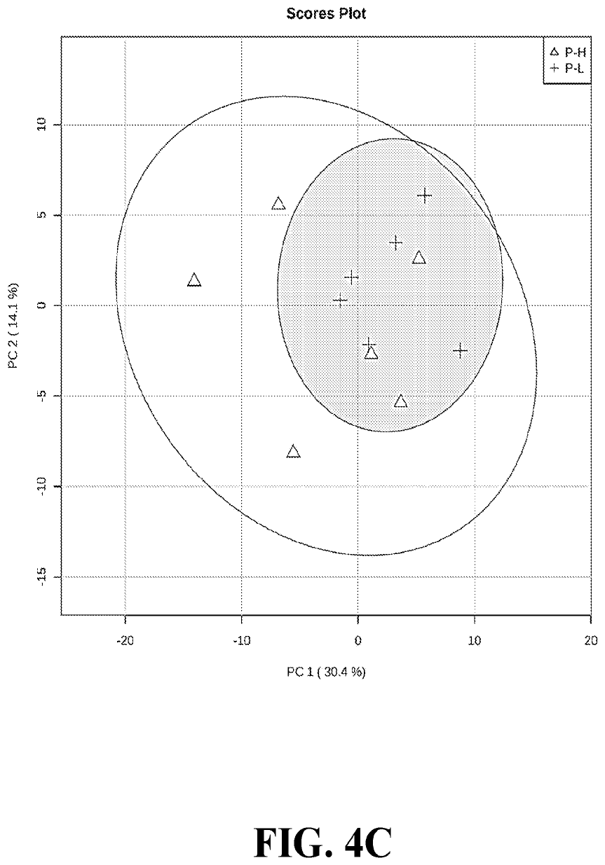

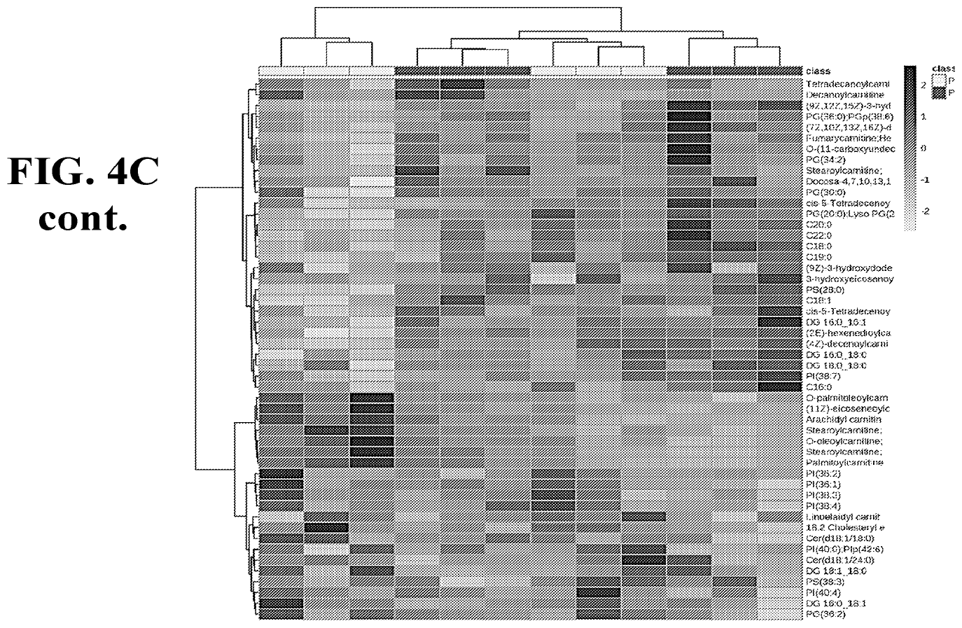

Heatmap and PCA analysis revealed distinct clusters in the F-H vs. P-L comparison for the lipid subsets A, B, and C (FIGS. 1A, 1B, and 1C). Distinct clusters based on F-L vs. P-H comparison were observed for the lipid subset B (FIG. 2B), but an overlap was observed for the lipid subsets A and C (FIGS. 2A and 2C). Heatmap and PCA analysis also revealed distinct clusters in the F-H vs. F-L comparison for the lipid subsets A and B (FIGS. 3A and 3B), but an overlap was observed for the lipid subset C (FIG. 3C). Distinct clusters based on P-H vs. P-L comparison were also observed for the lipid subsets B and C (FIGS. 4B and 4C), but an overlap was observed for the lipid subsets A and C (FIG. 4A).

Total TG and TG profile according to the carbon chain length and unsaturation degree is presented in Table 7. F-H steaks had a greater TG concentration than those from other treatments (P<0.001), while P-L steaks had less TG than F-L and P-H steaks (P<0.05), which did not differ from each other (P>0.05). F-H steaks also contained a greater concentration of 48-carbon TG than those from other treatments (P=0.041), while P-L animals had less 52-carbon (P=0.031) TG and more 56-(P=0.027), 58-(P=0.053), and 64-carbon (P=0.043) TG than other treatments. Moreover, P-L steaks contained a lower concentration of TG with 3 (P<0.001) sites of unsaturation and a higher concentration of TG with 0 (P<0.001), 1 (P<0.001), 5 (P=0.017), 6 (P=0.034), 7 (P=0.010), 8 (P<0.001), and 9 (P=0.022) sites of unsaturation than F-H and F-L steaks, but did not differ from P-H steaks in the concentration of TG with 1, 3 and 9 sites of unsaturation (P>0.05). Also, F-H steaks had less TG with up to 2 sites of unsaturation (P=0.012) and more TG with more than 3 sites of unsaturation (P<0.001) when compared with other treatments. There was no difference in total phospholipids and specific phospholipids among the treatments (P>0.05; Table 8).

TABLE 7
TG Data

| Characteristic Analyzed | F-H | F-L | P-H | P-L | SEM | P-value |
|---|---|---|---|---|---|---|
| Total triglycerides | 5.15^{a} | 3.82^{b} | 3.48^{b} | 2.23^{c} | 0.520 | <0.001 |
| (ng/μg muscle tissue) | | | | | | |
| Carbon no. (%) | | | | | | |
| 48 | 15.44^{a} | 13.55^{b} | 12.47^{b} | 13.29^{b} | 0.690 | 0.041 |
| 50 | 13.59 | 13.60 | 12.58 | 13.34 | 0.565 | 0.549 |
| 52 | 52.72^{a} | 53.16^{a} | 51.71^{a} | 49.20^{b} | 0.931 | 0.031 |
| 54 | 17.32^{b} | 18.29^{b} | 21.61^{a} | 21.49^{a} | 1.038 | 0.014 |
| 56 | 0.83^{b} | 1.22^{b} | 1.44^{b} | 2.33^{a} | 0.328 | 0.027 |
| 58 | 0.08^{b} | 0.12^{b} | 0.14^{b} | 0.26^{a} | 0.044 | 0.053 |
| 64 | 0.03^{b} | 0.04^{b} | 0.05^{b} | 0.09^{a} | 0.014 | 0.043 |
| Sites of unsaturation (%) | | | | | | |
| 0 | 1.41^{c} | 1.99^{bc} | 2.35^{b} | 3.08^{a} | 0.219 | <0.001 |
| 1 | 11.29^{c} | 13.63^{b} | 15.84^{a} | 16.19^{a} | 0.515 | <0.001 |
| 2 | 41.39 | 41.66 | 41.67 | 39.71 | 0.650 | 0.132 |
| 3 | 38.20^{a} | 34.65^{b} | 31.94^{c} | 30.41^{c} | 0.792 | <0.001 |
| 4 | 5.74 | 5.37 | 4.85 | 5.53 | 0.314 | 0.255 |
| 5 | 0.44^{b} | 0.69^{b} | 0.81^{b} | 1.33^{a} | 0.181 | 0.017 |
| 6 | 0.45^{b} | 0.63^{b} | 0.68^{b} | 1.14^{a} | 0.158 | 0.034 |
| 7 | 0.15^{b} | 0.25^{b} | 0.29^{b} | 0.49^{a} | 0.064 | 0.010 |
| 8 | 0.84^{c} | 1.00^{c} | 1.39^{b} | 1.83^{a} | 0.138 | <0.001 |
| 9 | 0.09^{b} | 0.14^{b} | 0.18^{ab} | 0.29^{a} | 0.042 | 0.22 |
| Grouped unsaturation (%) | | | | | | |
| Up to 2 | 52.68^{b} | 55.28^{a} | 57.52^{a} | 55.90^{a} | 0.930 | 0.012 |
| More than 3 | 45.90^{a} | 42.73^{b} | 40.13^{c} | 41.02^{bc} | 0.892 | <0.001 |

^{a-c}Values within a row with different superscripts differ significantly at P < 0.05.

TABLE 8
Phospholipids Data

| | | | | | | P- |
|---|---|---|---|---|---|---|
| Class, ng/μg muscle tissue | F-H | F-L | P-H | P-L | SEM | value |
| Phosphatidylcholine | 1.267 | 1.275 | 1.273 | 1.267 | 0.0031 | 0.146 |
| Phosphatidylethanolamine | 0.267 | 0.273 | 0.270 | 0.267 | 0.0032 | 0.405 |
| Phosphatidylglycerol | 0.030 | 0.028 | 0.030 | 0.027 | 0.0023 | 0.690 |
| Phosphatidylinositol | 0.065 | 0.077 | 0.077 | 0.062 | 0.0056 | 0.149 |
| Phosphatidylserine | 0.037 | 0.042 | 0.040 | 0.038 | 0.0030 | 0.683 |
| Sphingomyelin | 0.288 | 0.382 | 0.358 | 0.278 | 0.0517 | 0.423 |
| Total phospholipids | 1.953 | 2.072 | 2.043 | 1.937 | 0.0636 | 0.377 |

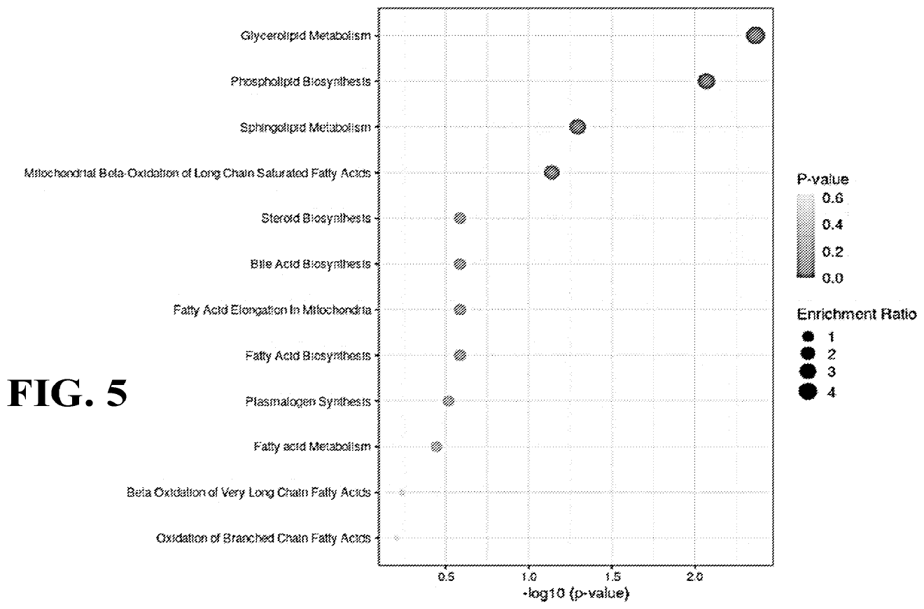

Quantitative enrichment analysis revealed that the main metabolic pathways affected by feeding regimen and growth rate (FIG. 5) were glycerolipid metabolism (P=0.004), phospholipid metabolism (P=0.009), sphingolipid metabolism (P=0.050) and mitochondrial beta-oxidation of long chain saturated fatty acids (P=0.073).

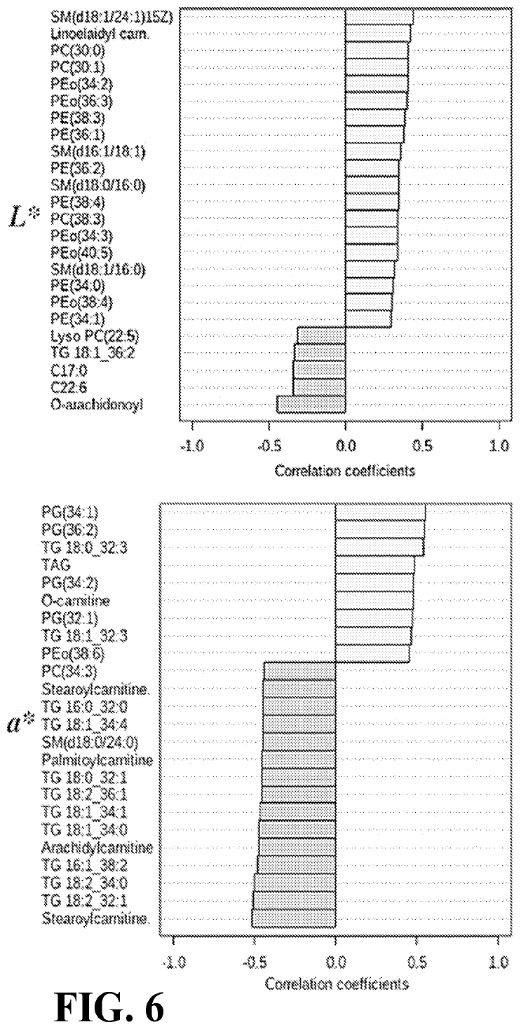

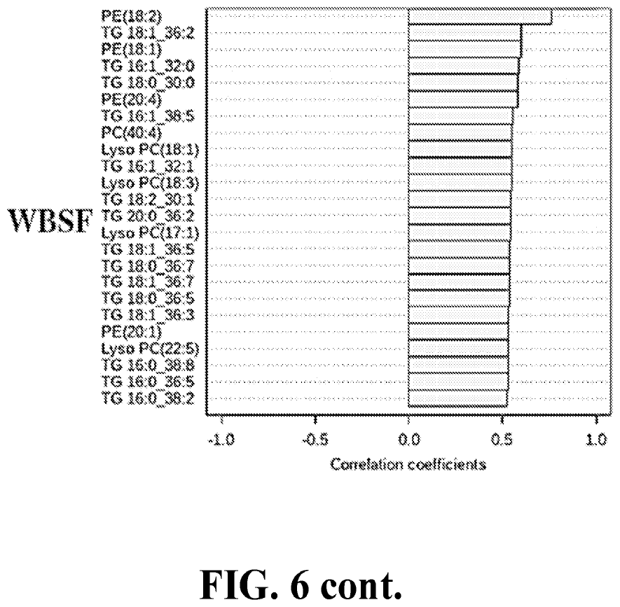

Correlation analysis revealed that seven lipids (3 AC, 2 PC, 1 PE, and 1 SM) had moderate (−0.4>r>0.4) and significant (P<0.05) correlation with L* (FIG. 6; Table 9), while 43 lipids (featuring 22 TG, 9 AC, and 5 PG) had moderate and significant correlations with a* (FIG. 6; Table 10) and 132 lipids (featuring 85 TG, 27 PC, and 15 PE) were moderately correlated with WBSF (FIG. 6; Table 11).

TABLE 9
Correlation with L*

| Lipid | Correlation | P-value |
|---|---|---|
| Linoelaidyl carnitine; | 0.42 | 0.039 |
| 9-12-Hexadecadienylcarnitine | | |
| O-arachidonoylcarnitine | −0.45 | 0.028 |
| O-linoleoylcarnitine | 0.42 | 0.039 |
| PC(30:0) | 0.41 | 0.048 |
| PC(30:1) | 0.41 | 0.048 |
| PEo(34:2) | 0.40 | 0.050 |
| SM(d18:1/24:1)15Z)) | 0.44 | 0.031 |

PC = phosphatidylcholine,
PE = phosphatidylethanolamine,
SM = sphingomyelin.

TABLE 10
Correlation with a*

| Lipid | Correlation | P-value |
|---|---|---|
| PG(34:1) | 0.55 | 0.005 |
| PG(36:2) | 0.54 | 0.006 |
| TG 18:0_32:3 | 0.54 | 0.007 |
| Stearoylcarnitine | −0.51 | 0.010 |
| TG 18:2_32:1 | −0.51 | 0.011 |
| TG 18:2_34:0 | −0.50 | 0.013 |
| TG | 0.48 | 0.017 |
| TG 16:1_38:2 | −0.48 | 0.018 |
| Arachidyl carnitine, O-[(9Z)- | −0.48 | 0.018 |
| 17-carboxyheptadec-9-enoyl]carnitine | | |
| PG(34:2) | 0.48 | 0.018 |
| O-(11-carboxyundecanoyl)carnitine | 0.48 | 0.019 |
| TG 18:1_34:0 | −0.48 | 0.019 |
| PG(32:1) | 0.47 | 0.021 |
| TG 18:1_34:1 | −0.47 | 0.021 |
| TG 18:1_32:3 | 0.46 | 0.023 |
| TG 18:2_36:1 | −0.46 | 0.025 |
| TG 18:0_32:1 | −0.45 | 0.026 |
| Palmitoylcarnitine, (5Z)- | −0.45 | 0.027 |
| 13-carboxytridec-5-enoylcarnitine | | |
| SM(d18:0/24:0) | −0.45 | 0.027 |
| PEo(38:6) | 0.45 | 0.027 |
| TG 18:1_34:4 | −0.45 | 0.027 |
| TG 16:0_32:0 | −0.45 | 0.028 |
| Hexadecanedioic acid | −0.45 | 0.029 |
| mono-L-carnitine ester | | |
| PC(34:3) | −0.44 | 0.030 |
| Dodecanoylcarnitine, | −0.44 | 0.030 |
| O-dodecanoylcarnitine | | |
| PG(36:1) | 0.44 | 0.031 |
| TG 18:0_34:2 | 0.44 | 0.031 |
| SM(d18:1/14:0) | 0.44 | 0.032 |
| DG 16:0_16:1 | 0.44 | 0.033 |
| TG 18:0_30:2 | 0.44 | 0.033 |
| TG 16:1_36:0 | −0.44 | 0.033 |
| Fumarycarnitine, | 0.43 | 0.034 |
| Hexanoylcarnitine | | |
| TG 18:0_36:1 | −0.43 | 0.034 |
| TG 18:2_36:0 | −0.43 | 0.035 |
| TG 16:0_38:0 | −0.43 | 0.036 |
| TG 16:1_38:0 | −0.43 | 0.038 |
| Tetradecanoylcarnitine, | −0.42 | 0.041 |
| O-tetradecanoylcarnitine | | |
| TG 16:1_38:3 | −0.42 | 0.043 |
| SM(d16:1/22:1) | −0.41 | 0.044 |
| TG 16:0_34:3 | 0.41 | 0.047 |
| Decanoylcarnitine | −0.41 | 0.047 |
| TG 16:0_36:0 | −0.41 | 0.047 |
| TG 18:2_34:1 | −0.41 | 0.049 |

TG = triglyceride,
PG = phosphatidylglycerol,
SM = sphingomyelin,
PE = phosphatidylethanolamine,
PC = phosphatidylcholine, and
DG = diglyceride.

TABLE 11
Correlation with WBSF

| Lipids | Correlation | P-value |
|---|---|---|
| PE(18:2), Lyso PE(18:2) | 0.76 | 0.001 |
| TG 18:1_36:2 | 0.60 | 0.002 |
| PE(18:1), Lyso PE(18:1) | 0.60 | 0.002 |
| TG 16:1_32:0 | 0.59 | 0.002 |
| TG 18:0_30:0 | 0.58 | 0.003 |
| PE(20:4), Lyso PE(20:4) | 0.58 | 0.003 |
| TG 16:1_38:5 | 0.56 | 0.005 |
| PC(40:4) | 0.55 | 0.005 |
| Lyso PC(18:1) | 0.55 | 0.005 |
| TG 16:1_32:1 | 0.55 | 0.005 |
| Lyso PC(18:3) | 0.55 | 0.005 |
| TG 18:2_30:1 | 0.55 | 0.006 |
| TG 20:0_36:2 | 0.54 | 0.006 |
| Lyso PC(17:1) | 0.54 | 0.006 |
| TG 18:1_36:5 | 0.54 | 0.006 |
| TG 18:0_36:7 | 0.54 | 0.007 |
| TG 18:1_36:7 | 0.54 | 0.007 |
| TG 18:0_36:5 | 0.54 | 0.007 |
| TG 18:1_36:3 | 0.53 | 0.007 |
| PE(20:1), Lyso PE(20:1) | 0.53 | 0.007 |
| Lyso PC(22:5) | 0.53 | 0.008 |
| TG 16:0_38:8 | 0.53 | 0.008 |
| TG 16:0_36:5 | 0.53 | 0.008 |
| TG 16:0_38:2 | 0.53 | 0.008 |
| TG 16:1_36:5 | 0.53 | 0.008 |
| PE(20:3), Lyso PE(20:3) | 0.53 | 0.008 |
| TG 20:0_34:8 | 0.53 | 0.008 |
| TG 18:0_36:6 | 0.52 | 0.008 |
| TG 18:0_38:7 | 0.52 | 0.009 |
| TG 16:1_42:4 | 0.52 | 0.009 |
| TG 20:0_32:2 | 0.52 | 0.009 |
| TG 20:4_30:2 | 0.52 | 0.009 |
| TG 20:0_36:0 | 0.52 | 0.009 |
| TG 18:1_36:1 | 0.52 | 0.010 |
| TG 16:0_38:4 | 0.52 | 0.010 |
| PC(40:2) | 0.52 | 0.010 |
| TG 16:1_42:8 | 0.51 | 0.010 |
| TG 20:0_34:7 | 0.51 | 0.010 |
| TG 20:4_32:1 | 0.51 | 0.010 |
| TG 16:1_38:4 | 0.51 | 0.010 |
| TG 16:0_36:6 | 0.51 | 0.010 |
| TG 20:0_34:0 | 0.51 | 0.011 |
| TG 20:0_32:1 | 0.51 | 0.011 |
| TG 18:1_38:5 | 0.51 | 0.011 |
| TG 18:2_32:0 | 0.51 | 0.011 |
| TG 18:1_38:3 | 0.51 | 0.011 |
| TG 18:1_40:8 | 0.51 | 0.011 |
| TG 18:2_38:1 | 0.51 | 0.011 |
| TG 16:1_40:1 | 0.51 | 0.011 |
| PC(34:4) | 0.51 | 0.011 |
| TG 18:1_36:4 | 0.51 | 0.012 |
| TG 18:2_36:4 | 0.51 | 0.012 |
| TG 18:1_38:6 | 0.50 | 0.012 |
| SM(d18:0/24:0) | 0.50 | 0.012 |
| TG 20:4_30:0 | 0.50 | 0.012 |
| TG 18:2_38:2 | 0.50 | 0.013 |
| TG 20:0_36:1 | 0.50 | 0.013 |
| TG 16:1_36:3 | 0.50 | 0.013 |
| TG 16:1_40:5 | 0.50 | 0.013 |
| TG 18:0_38:6 | 0.50 | 0.014 |
| TG 20:4_36:1 | 0.50 | 0.014 |
| TG 18:1_36:0 | 0.50 | 0.014 |
| TG 20:0_32:0 | 0.50 | 0.014 |
| TG 16:0_40:0 | 0.49 | 0.014 |
| TG 20:4_32:0 | 0.49 | 0.014 |
| TG 18:0_36:0 | 0.49 | 0.014 |
| TG 16:1_34:3 | 0.49 | 0.014 |
| TG 18:2_30:0 | 0.49 | 0.014 |
| TG 20:0_36:3 | 0.49 | 0.015 |
| TG 18:1_32:0 | 0.49 | 0.015 |
| PE(34:3) | 0.49 | 0.015 |
| PE(22:4), Lyso PE(22:4) | 0.49 | 0.015 |
| Lyso PC(20:0) | 0.49 | 0.015 |
| TG 16:1_40:7 | 0.49 | 0.015 |
| PC(42:9), PCo(42:2) | 0.49 | 0.016 |
| PC(36:7) | 0.49 | 0.016 |
| PE(36:0), PEp(36:6) | 0.49 | 0.016 |
| PEo(36:2) | 0.48 | 0.016 |
| TG 18:0_34:5 | 0.48 | 0.016 |
| Lyso PC(20:1) | 0.48 | 0.017 |
| TG 16:1_40:0 | 0.48 | 0.017 |
| TG 20:4_36:0 | 0.48 | 0.017 |
| SM(d18:2/20:1) | 0.48 | 0.017 |
| TG 20:4_36:2 | 0.48 | 0.017 |
| PC(40:10), PCo(40:3) | 0.48 | 0.018 |
| TG 18:2_34:2 | 0.48 | 0.018 |
| TG 18:1_38:4 | 0.48 | 0.018 |
| Lyso PC(20:5) | 0.48 | 0.018 |
| TG 18:2_36:3 | 0.48 | 0.018 |
| Lyso PC(17:0) | 0.48 | 0.019 |
| PE(38:8), PEo(38:1) | 0.48 | 0.019 |
| TG 16:0_38:3 | 0.47 | 0.019 |
| PC(38:4) | −0.47 | 0.020 |
| Lyso PC(15:0) | 0.47 | 0.021 |
| PE(38:9), PEo(38:2) | 0.47 | 0.021 |
| TG 20:0_44:2 | 0.47 | 0.021 |
| TG 20:4_34:1 | 0.47 | 0.022 |
| PC(32:5) | 0.47 | 0.022 |
| TG 18:0_34:4 | 0.46 | 0.023 |
| TG 16:1_40:4 | 0.46 | 0.023 |
| PC(42:5) | 0.46 | 0.023 |
| TG 18:1_38:1 | 0.46 | 0.023 |
| TG 16:1_40:2 | 0.46 | 0.024 |
| TG 18:1_38:7 | 0.46 | 0.024 |
| PC(42:10), PCo(42:3) | 0.46 | 0.024 |
| TG 18:0_38:2 | 0.46 | 0.024 |
| TG 16:0_36:4 | 0.45 | 0.027 |
| TG 18:0_34:0 | 0.45 | 0.028 |
| PE(18:3), Lyso PE(18:3) | 0.44 | 0.030 |
| PE(20:5), Lyso PE(20:5) | 0.44 | 0.030 |
| PC(40:0), PCp(42:6) | 0.44 | 0.030 |
| PI | −0.44 | 0.031 |
| TG 18:2_32:4 | 0.44 | 0.031 |
| Lyso PC(19:1) | 0.44 | 0.032 |
| TG 18:1_38:0 | 0.44 | 0.032 |
| PE(18:0), Lyso PE(18:0) | 0.44 | 0.033 |
| TG 16:1_34:2 | 0.44 | 0.033 |
| Lyso PC(16:1) | 0.43 | 0.034 |
| Lyso PC(19:0) | 0.43 | 0.035 |
| TG 18:2_32:1 | 0.43 | 0.035 |
| TG 18:0_30:3 | 0.43 | 0.037 |
| SM(d18:1/26:1)17Z)) | 0.42 | 0.039 |
| PC(44:7) | 0.42 | 0.040 |
| TG 16:1_40:8 | 0.42 | 0.040 |
| PC(40:8), PCo(40:1) | 0.42 | 0.041 |
| PC(40:1) | 0.42 | 0.041 |
| PC(42:7), PCo(42:0) | 0.42 | 0.042 |
| C16:0 | 0.42 | 0.043 |
| TG 16:1_36:2 | 0.41 | 0.045 |
| TG 16:1_38:3 | 0.41 | 0.048 |
| PE(20:2), Lyso PE(20:2) | 0.41 | 0.048 |
| TG 20:0_34:1 | 0.41 | 0.048 |

TG = triglyceride,
SM = sphingomyelin,
PE = phosphatidylethanolamine,
PC = phosphatidylcholine, and
PI = phosphatidylinositol.

Intramuscular fat mostly consists of structural lipids, phospholipids, and TG, which is the result of the intake of fatty acids through the diet, de novo fatty acid biosynthesis, TG formation, and TG degradation. Listrat et al., How muscle structure and composition influence meat and flesh quality, Science World Journal (2016); Nümberg et al., Effects of growth and breed on the fatty acid composition of the muscle lipids in cattle, Eur. Food Research Technology 208:332-335 (1999). IMF content is mainly affected by cattle breed, diet composition and animal's age, but can also depend on the muscle growth rate. Nümberg et al. (1999), supra; Mwangi et al. (2019), supra; Wicks et al. (2019), supra; and Hocquette et al., Contribution of genomics to the understanding of physiological functions, J Physiological Pharmacology 60:5-16 (2009).

In the present study, feeding regimen and growth rate impacted IMF deposition, mainly because F-H animals had higher IMF deposition than P-L animals (Table 2). However, under a similar growth rate, F-L and P-H animals had similar IMF deposition suggesting diet was the main driver of IMF deposition, which aligns with published reports of higher IMF deposition in animals fed high-concentrate diets (higher ADG) when compared to those fed high-quality forages (lower ADG). Koch et al., Postweaning Exposure to High Concentrates versus Forages Alters Marbling Deposition and Lipid Metabolism in Steers, Meat Muscle Biology 3:244-253 (2019). This is most likely due to increases in propionate from starch-based diets, which is the primary lipid precursor in intramuscular adipose tissue compared to acetate from a grass-based diet. Smith & Crouse, Relative contributions of acetate, lactate and glucose to lipogenesis in bovine intramuscular and subcutaneous adipose tissue, J Nutrition 114:792-800 (1984). This may also help explain the higher IMF deposition in feedlot animals fed ad libitum since there would be even greater amounts of propionate compared to those fed in feedlots to attain slower growth rates comparable to P-L cattle. Therefore, feeding regimen or dietary component (grain vs. grass) was the main factor altering IMF deposition.

Here, F-H steaks had greater total TG deposition than F-L and P-L animals, while P-H steaks had greater TG deposition than P-L cattle. In addition, F-H steaks had a higher amount of unsaturated TG, which was mainly due to TG with 3 double bonds compared to other treatments (Table 7). These differences in TG profile can be partially explained by the different requirements of TG mobilization in order to release fatty acids so that they are oxidized in the mitochondrial matrix as a source of energy for animal growth, which may be supported by the impact of treatments on mitochondrial β-oxidation of long chain fatty acids pathway.

Ladeira et al. reported that higher levels of glycerol in the muscle, as suggested in F-H animals in the present study, may be indicative of TG hydrolysis, which may support mitochondrial oxidation as lower levels of free carnitine may reflect utilization for long chain fatty acid transport into the mitochondria. Ladeira et al., Effect of increasing levels of glycerin on growth rate, carcass traits and liver gluconeogenesis in young bulls, Aminal Feed Science Technology 219:241-248 (2016); Ladeira et al., Review: Nutrigenomics of marbling and fatty acid profile in ruminant meat, Animal 12: S282-S294 (2018). Here, F-H animals had higher hexadecanedioic acid mono-L-carnitine ester and stearoylcarnitine concentration than P-L animals (Table 3), which aid in the mechanism whereby long-chain fatty acids are transferred from the cytosol to the mitochondrial matrix to undergo β-oxidation. Moreover, although F-L animals had greater concentrations of nine different acyl-carnitines, including elaidic carnitine, O-oleoylcarnitine, palmitoylcarnitine and cervonyl carnitine, than P-H animals, they had similar TG deposition and profile, except for TG with 1 and 3 sites of unsaturation, which may be attributed to differences in the feeding regime. Therefore, these results may indicate that growth rate had the greatest impact at altering TG deposition and TG profile.

In addition to the neutral lipids primarily consisting of TG, IMF is also composed of polar lipids containing mostly phospholipids. Legako et al., Effects of USDA beef quality grade and cooking on fatty acid composition of neutral and polar lipid fractions, Meat Science 100:246-255 (2015). Scollan et al. reported that the overall fat content of the animal and muscle has an important impact on proportionate fatty acid composition because of the different fatty acid composition of TG and phospholipids. Scollan et al., Enhancing the nutritional and health value of beef lipids and their relationship with meat quality, Meat Science 97:384-394 (2014). Wood et al. stated that cattle with lower levels of IMF are expected to produce meat with a higher amount of unsaturated fatty acids, which are restricted almost exclusively to the phospholipid fraction. Wood et al., Effects of fatty acids on meat quality: A review, Meat Science 66:1-32 (2004). Moreover, Bressan et al. reported that the IMF profile largely depends on the finishing system, in which grain-fed animals have more saturated fatty acids while grass-fed animals have more unsaturated fatty acids. Bressan et al., Differences in intramuscular fatty acid profiles among Bos indicus and crossbred Bos taurus×Bos indicus bulls finished on pasture or with concentrate feed in Brazil, Italian J Animal Science 15:10-21 (2016).

In the present study, despite the higher deposition of IMF and unsaturated TG in F-H steaks compared to other treatments, feeding strategies did not change the concentration of phospholipids or total phospholipid deposited in lean muscle. However, phospholipid profiles within classification were impacted, wherein P-L steaks had greater amounts of most PC, PE and SM compared to F-H steaks, while P-H beef had more PC, PE and SM compared to F-L steaks (Table 9). These data may be explained by changes in the IMF composition caused by the impact of the treatments on the glycerolipid metabolism pathway (FIG. 5). Moreover, PC, PE and SM did not present a clear abundance direction when comparing growth rate within the feeding regimen (F-H vs. F-L and P-H vs. P-L). Differences in phospholipid profiles across treatments may be partially explained by the impact of the treatments on the phospholipid biosynthesis and sphingolipid metabolism pathways (FIG. 5). Therefore, these results may indicate that feeding regimen (grain vs. pasture) seemed to be the main factor that altered phospholipid profile.

Some MRM from the PC, PE, SM and AC classes were positively correlated with beef lightness (L*) (Table 9). Specifically, SM(d18:1/24:1 (15Z)) was the most correlated MRM with L*, in addition to being one of the top 25 MRM clustered to distinguish F-H vs. P-L and F-H vs. F-L comparisons (FIGS. 1B and 3B, respectively). Interestingly, although SM(d18:1/24:1 (15Z)) content was 1.07- and 1.14-fold higher in F-L and P-L when compared to F-H, respectively, lower L* values were observed in F-L and P-L steaks. The SM(d18:1/24:1 (15Z)) is a type of sphingolipid found in animal cell membranes that helps prevent damage to the cell structure. Gault et al., An overview of sphingolipid metabolism: from synthesis to breakdown, Advanced Exp. Medical Biology 688:1-23 (2010). Hughes et al. reported that the lipid component of cell membranous structures is believed to be partially responsible for light scattering; thus, microstructural components in muscles cells dictate light scattering and beef lightness. Hughes et al., Meat color is determined not only by chromatic heme pigments but also by the physical structure and achromatic light scattering properties of the muscle, Compr. Rev. Food Science & Food Safety 19:44-63 (2020).

The results of this study suggest that modifications to the cell structure, such as the fluidity of membrane increasing the permeability, mainly in F-H steaks when compared to other treatments, contributes to the light scattering process, in which light is diffused or deflected by collisions with particles of the medium that it transverses. Similarly, Koch et al. observed lighter steaks from feedlot-fed cattle with higher marbling scores and lipid content within the muscle, since greater IMF deposition alters the muscle structure. Koch et al. (2019), supra; Valenzuela et al., Adipose invasion of muscle in Wagyu cattle: Monitoring by histology and melting temperature, Meat Science 163:108063 (2020). Moreover, Bate-Smith reported that light scattering is also partially dependent on the texture of the meat surface, which agrees with the WBSF data found in this study, where pasture-fed animals produced less tender meat and lower L* values than feedlot-fed animals. Bate-Smith, Observations on the phand related properties of meat, J Soc. Chem. Ind. 67:83-90 (1948).

In the present study, several TG, AC, and PG were correlated with a* (redness) (Table 10), in addition to being among the top 25 MRM clustered, in their subset lipid, to distinguish F-H vs. P-L comparison (FIG. 1). The direction of the correlation was observed to be conditional to the MRM concentration in the F-H and P-L groups than in the F-L and P-H groups, where the higher concentration in F-H compared to P-L group, contributed to a greater correlation. Overall, most of the TG and AC were negatively correlated with a*, which may suggest that the accumulation of compounds by oxidation of unsaturated fatty acids and meat phospholipids is correlated with myoglobin oxidation in fresh beef. Faustman et al., Myoglobin and lipid oxidation interactions: Mechanistic bases and control, Meat Science 86:86-94 (2010). Ramanathan et al. reported that changes in oxymyoglobin and a* values appeared to be driven by the oxidation of unsaturated fatty acids in phospholipids and TG. Ramanathan et al. (2020), supra.

Specifically, in this study, PG (34:1) and PG (36:2) were the most positively correlated MRMs with redness, which is in good agreement with their greater abundance in F-H steaks compared to P-L steaks (Table 3). PG (34:1) and PG (36:2) are precursors of cardiolipins, which are plentiful in the intermembrane of mitochondria and suggestive of increased mitochondrial activity and presumably critical in achieving and maintaining a bright cherry-red in fresh beef. Chen et al., Phosphatidylglycerol Incorporates into Cardiolipin to Improve Mitochondrial Activity and Inhibits Inflammation, Science Rep. 8:1-14 (2018); Ramanathan & Mancini, Role of Mitochondria in Beef Color: A Review, Meat Muscle Biology 2:309-320 (2018). Therefore, changes in TG, AC, and PG profiles promoted by increased growth rate may contribute to increased redness in F-H steaks mainly through greater unsaturated fatty acid oxidation and a corresponding increase in mitochondrial activity.

Several TG, PC and PE were positively correlated with WBSF (Table 11), in addition to being among the top 25 MRM clustered, in their subset lipid, to distinguish F-H vs. P-L and F-L vs. P-H comparisons (FIGS. 1 and 2, respectively). The WBSF correlation was observed to be conditional to feeding regime, where the higher TG, PC and PE concentration in pasture-fed animals compared to feedlot-fed animals, the higher correlation observed. Specifically, PE (18:2) was the most positively correlated MRM with WBSF, which belongs to the PE class that is implicated in cellular apoptosis via mitochondrial permeability transition that was initiated by reactive oxygen species. Kaku et al., Diarachidonoylphosphoethanolamine induces necrosis/necroptosis of malignant pleural mesothelioma cells, Cell. Signal 27:1713-1719 (2015).

Oak et al. reported that PE degradation generates products that accelerate membrane lipid peroxidation, causing oxidative stress to cells, which positively affects the development of tenderness. Oak et al., Synthetically prepared Amadori-glycated phosphatidylethanolamine can trigger lipid peroxidation via free radical reactions, FEBS Letters 481:26-30 (2000); Gagaoua et al., Coherent correlation networks among protein biomarkers of beef tenderness: What they reveal, J Proteomics 128:365-374 (2015). These authors hypothesized that PC hydrolysis at the first stage of postmortem apoptosis may invert membrane polarity, which will induce changes in membrane fluidity, thus increasing the permeability to ions such as Ca²⁺, increasing μ-calpain activity, and increasing beef tenderness. The results of the present study suggest that steaks from feedlot-fed animals underwent higher postmortem degradation of PC and PE than pasture-fed animals. Therefore, although PC and PE concentration were not altered (Table 8), their profiles were modified by feeding regimen and contributed to the tenderization of beef.

There are several possible explanations for the positive effect of lipids on tenderness, including the presence of TG within the perimysium in fat cells, which might have a physical effect in the process of tenderization by separating muscle fiber bundles and opening the muscle structure. Wood et al., Effects of fatty acids on meat quality: A review, Meat Science 66:1-32 (2004). Ouali et al. (2013) suggested more active participation of lipids in the tenderizing process through contributing to energy production in the first hours after slaughter, mainly via AMP-activated protein kinase. Ouali et al., Biomarkers of meat tenderness: Present knowledge and perspectives in regards to our current understanding of the mechanisms involved, Meat Science 95:854-870 (2013); Scheffler and Gerrard, Mechanisms controlling poor quality development: The biochemistry controlling postmortem energy metabolism, Meat Science 77:7-16 (2007). Polati et al. reported an increase of β-hydroxyacyl CoA-dehydrogenase, a member of the β-oxidation of lipids, which is an indicative of TG degradation and may be used as a biomarker of beef tenderness. Polati et al., Proteomic changes involved in tenderization of bovine Longissimus dorsi muscle during prolonged aging, Food Chemistry 135:2052-2069 (2012). Therefore, the main reason for the changes in TG and phospholipid profiles that were caused by the feeding regimen contributed to the beef tenderness is not completely clear, but results suggest that TG and phospholipid profiles, and their degradation and signaling contribute to the development of tender beef.

This study elucidated the effects of feeding regimen and growth rate on beef color and tenderness, and on the profile of muscle lipids, using a lipidomics approach based on MRM-profiling. Lipid content and profile differed to feeding strategies, which were related to L*, a*, and tenderness. Overall, results indicate that feeding regimen is the main factor that is responsible for altering IMF deposition and phospholipid profiles, which contribute to the development of beef lightness and tenderness. Moreover, growth rate was the main factor that affected TG deposition and profile, which was positively correlated with the redness of the beef.

### Example 4

### Materials & Methods of pH Studies

Twelve carcasses classified as normal (pHu<5.8; n=6) or high (pHu≥6.2; n=6) pHu beef from pasture-finished Nellore (Bos Indicus) bulls, ranging from 30 to 35 months of age (4 to 6 permanent incisors teeth), were obtained at a commercial facility (JBS S. A., Lins, Sao Paulo, Brazil). Samples (˜ 30 g) were excised from the LT muscle adjacent to the 10th and 11th ribs immediately after skinning (approximately 30 minutes postmortem) and at 44 h postmortem. All samples were immediately snap frozen in liquid nitrogen and stored at −80° C. for pHu determination and lipidome and metabolome analyses.

Muscle pHu was determined using the iodoacetate method as described by Bendall, The structure and function of muscle. H. Bournes (in Post mortem changes in muscle): 227-274, New York: Academic Press (1973). Briefly, LT muscle samples were powdered in liquid nitrogen for 44 hours, added to a buffer containing 5 mM sodium iodoacetate and 150 mM KCl (pH 7.0) at a 1:8 ratio (wt/vol), and homogenized. Muscle homogenates were centrifuged at 13,000×g for 5 minutes at room temperature, equilibrated to 25° C. and measured using a portable digital pH meter (Sentron, SI600 model, VD Leek, Netherlands).

Approximately 50 mg of each muscle sample (n=6 per pH classification) collected at 30 minutes and 44 hours postmortem were ground in liquid nitrogen for lipid extraction using a method reported by Bligh & Dryer, A rapid method of total lipid extraction and purification, Canadian J Biochemistry & Physiology 37 (8): 911-917 (1959). Briefly, 300 μL of ultrapure water was added to each microfuge tube containing a muscle sample, and mixtures were gently mixed to promote cell lysis. Then, 250 μL of chloroform and 450 μL of methanol were added and mixed by pipetting for 15 seconds. Solution was incubated at 4° C. for 15 minutes prior to the addition of 250 μL of chloroform and 250 μL of ultrapure water and centrifugated at 16,000×g for 10 minutes, forming a two-phase solution where the bottom phase contained the lipids (organic phase). The organic phase was then transferred to a new tube, dried using a centrifugal vacuum concentrator (Genevac™ miVac, Genevac LTD., Ipswich, UK) and then stored at −80° C. until further analysis.

### Example 5

### pH MRM-Profiling

Targeted lipid profiling was performed using discovery MRM-profiling methods and instrumentation as recently reviewed by Xie et al. (2021), supra, and substantially as set forth in Example 3 above.

Specifically, dried lipid extracts were diluted in 50 μL of methanol/chloroform 3:1 (v/v) and 250 μL of injection solvent (acetonitrile/methanol/300 mM ammonium acetate 3:6.65:0.35 [v/v/v]) to obtain a stock solution. Mass spectrometry data were acquired by flow-injection (no chromatographic separation) from 8 μL of stock solution that was diluted 300-fold in injection solvent and spiked with EquiSPLASH™ LIPIDOMIX® Quantitative Mass Spec Internal Standard (0.1 ng/μL) prior to being delivered to the ESI source of an Agilent 6410 triple quadrupole mass spectrometer (Agilent Technologies, Santa Clara, CA, US) using a micro-autosampler (G1377A). A capillary pump was coupled to the autosampler and operated at a flow rate of 10 μL/min and pressure of 150 bar. Capillary voltage of the instrument was 4 kV, and the gas flow 5.1 L/min at 300° C. The MRM profiling method was used to profile 1366 MRMs related to lipids, from 11 lipid classes. The MRM set included 152 PE, 62 AC, 57 CE, 121 PC, 27 SM, 148 PI, 152 PG, 148 PS, 36 FFA, 80 CER, and 383 TAG. TAGs were profiled using parent ions and a product ion related to the presence of specific fatty acyl residues (C16:0, C16:1, C18:0, C18:1, C18:2, and C20:4). The assigned short hand identity, with TAG 16:0_36:1 as an example, starts with class (TAG) followed by the fatty acyl chain related to the product ion (16:0) and ends with the sum of the carbon: unsaturation number related to other two fatty acyl chains at the TAG (e.g. 36:1), as recommended by the Lipid Maps nomenclature group (Liebisch et al. (2020), supra). Phospholipids were identified by their class (PG, PS, PI, PE, or PC), the number of carbon atoms between both esterified fatty acids, and the number of carbon-carbon double bonds present in the molecule, e.g. PE (34:4). Ion intensity data of each MRM per sample were obtained using in-house scripts that were used for subsequent analysis.

### Example 6

### pH Metabolome Analysis

Approximately 500 mg of each muscle sample collected at 30 minutes postmortem (n=5 per pHu classification) and 44 hours postmortem (n=6 per pHu classification) were ground in liquid nitrogen and mixed with 3.5 mL of a cold methanol/chloroform/water solution (2:2:1 v/v) to extract the polar metabolite as previously described by Beckonert et al., Metabolic profiling, metabolomic and metabonomic procedures for NMR spectroscopy of urine, plasma, serum and tissue extracts, Nature Protocols 2 (11): 2692-2703 (2007). Detailed information on polar metabolites extraction can be found in Antonelo et al., Metabolites and metabolic pathways correlated with beef tenderness, Meat & Muscle Biology 4 (1): 1-9 (2020); Cônsolo et al., Selection for growth and precocity alters muscle metabolism in Nellore cattle, Metabolites 10 (2): 58-68 (2020). Briefly, a supernatant volume of 0.5 mL was transferred to microfuge tubes and freeze-dried (Itasul Import and Instrumental Technical Ltda, Porto Alegre, Brazil).

The freeze-dried residue was reconstituted in 600 μL of 100 mM phosphate buffer which contained a nuclear magnetic resonance (NMR) internal standard as previously described by Antonelo et al. (2020), supra and Cônsolo et al. (2020), supra. Supernatants (600 μL) were then transferred to standard 5×178 mm thin-walled NMR tubes (VWR International, US) and stored until analyses.

One dimensional proton nuclear magnetic resonance (1D ¹H NMR) spectra were acquired at 300 K using a Bruker Avance 14.1 T spectrometer (Bruker Corporation, Karlsruhe, Baden-Württemberg, Germany) at 600.13 MHz for ¹H, using a 5 mm Broadband Observe (BBO) probe. Detailed information on NRM spectroscopy can be found in Antonelo et al. (2020), supra and Cônsolo et al. (2020), supra. The following acquisition parameters were used: 13.05 us for the 90° pulse, 0.5 seconds relaxation delay, 64 scans, 3.89 seconds acquisition time, and 10.03 ppm spectral width.

The 1D ¹H NMR spectra were processed with a 0.3 Hz line broadening using TopSpin™ 3.6.1 software (Bruker, Biospin, Germany). Phase and baseline correction were performed manually using the Chenomx NMR Suite 8.4 (Chenomx Inc., Edmonton, Canada), and the pH was calibrated using the resonances of imidazole. Spectra were referenced to the 3-(trimethylsilyl)-1-propanesulfonic acid sodium salt (DSS) methyl peak at 0.00 ppm, which was used as an internal standard for quantitation. Metabolites were quantified in the 1D ¹H NMR spectra of beef extracts using the Profiler module of Chenomx NMR Suite Professional software with an in-built 1D spectral library. Quantitation was based on comparing the area of selected metabolite peaks and the area under the DSS methyl peak, which corresponds to a known concentration of 1.0 mM in each sample.

### Example 7

### pH Lipidome Analysis

To minimize MRM noise, only ion intensities greater than 1.3-fold the ion intensity of blanks were considered for statistical analyses. Relative ion intensities were calculated for each MRM by dividing its ion intensity by the sum of all ion intensities across each sample. MRMs were then assigned to one of two lipid classifications: method 1) CER, PC, PE, PG, PI, PS and SM; and method 2) AC, DG, FFA and TAG. Using these designations, the following comparisons were made: a) normal pHu versus high pHu at 30 minutes postmortem; b) normal pHu versus high pHu at 44 hours postmortem; c) 30 minutes versus 44 hours postmortem-normal pHu; and d) 30 minutes versus 44 hours postmortem-high pHu. Treatment effects on TAG distribution in each sample were analyzed using three distinct groupings for Student's t-test analysis: 1) total number of carbons; 2) total number of unsaturations; and 3) total number of unsaturations grouped into: 0 (unsaturated), up to 2 unsaturations, and ≥3 unsaturations. The relative ion intensities were uploaded to Metaboanalyst 5.0 (Chong et al. (2019), supra). Relative ion intensity data were normalized by autoscaling and statistical analysis was performed using Student's t-test. Moreover, internal standards were used to obtain the relative amount of total TAG, total phospholipid, and phospholipid profiles according to class. Differences were considered statistically significant when P≤0.05.

PCA was performed, and clustered heatmaps were plotted using the differentially abundant lipids and Ward's method was used to cluster samples. Quantitative enrichment analysis was performed using lipid quantification data sets for each treatment. Compound names were standardized according to Kyoto Encyclopedia of Genes and Genomes (KEGG), Human Metabolome Database (HMDB) or PubChem ID and the library chosen was the Small Molecule Pathway Database (SMPDB).

The metabolite concentration table was uploaded to MetaboAnalyst 5.0 (Chong et al. (2019), supra) and data were Pareto-scaled prior to analysis.

Statistical analysis was performed using Student's t-test. Differences were considered statistically significant when P≤0.05 and marginally significant when 0.05≤P≤0.10. PCA and clustered heatmaps were also generated. In addition, pathway analysis was performed to identify the most relevant pathways associated with the identified metabolites using a web-based analysis module that is based on the KEGG database. Through pathway analysis, metabolic pathways related to the identified metabolites were mapped based on P-values (P≤0.10) and pathway impacts (PI≥0.10).

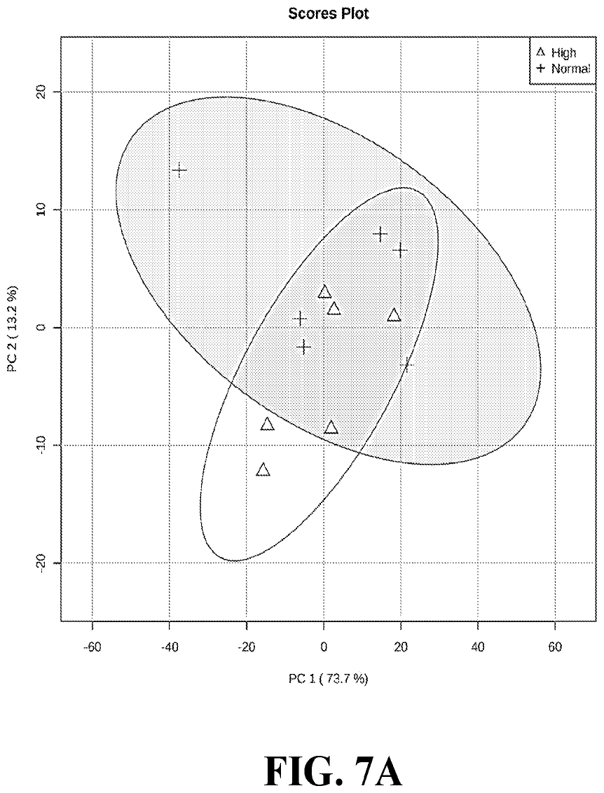

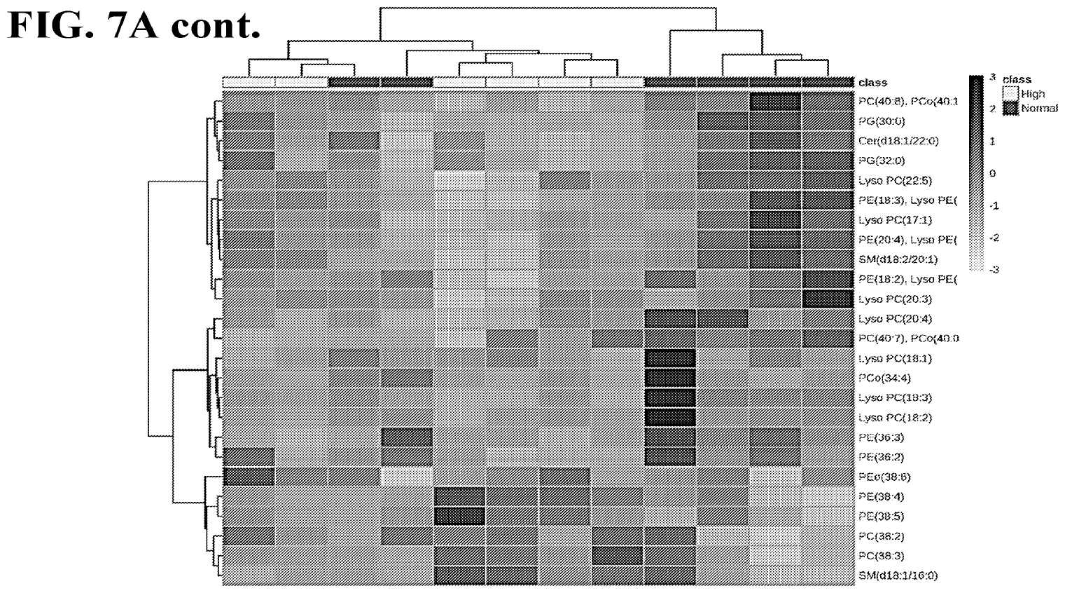

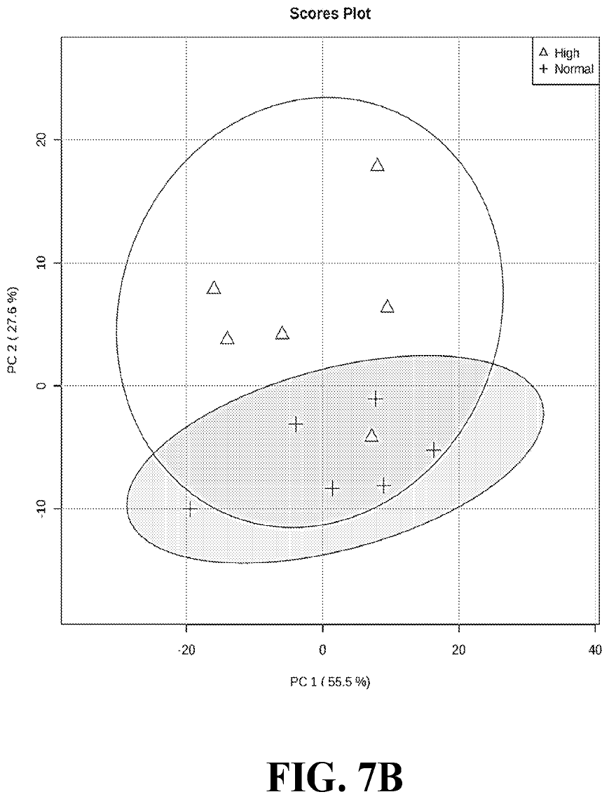

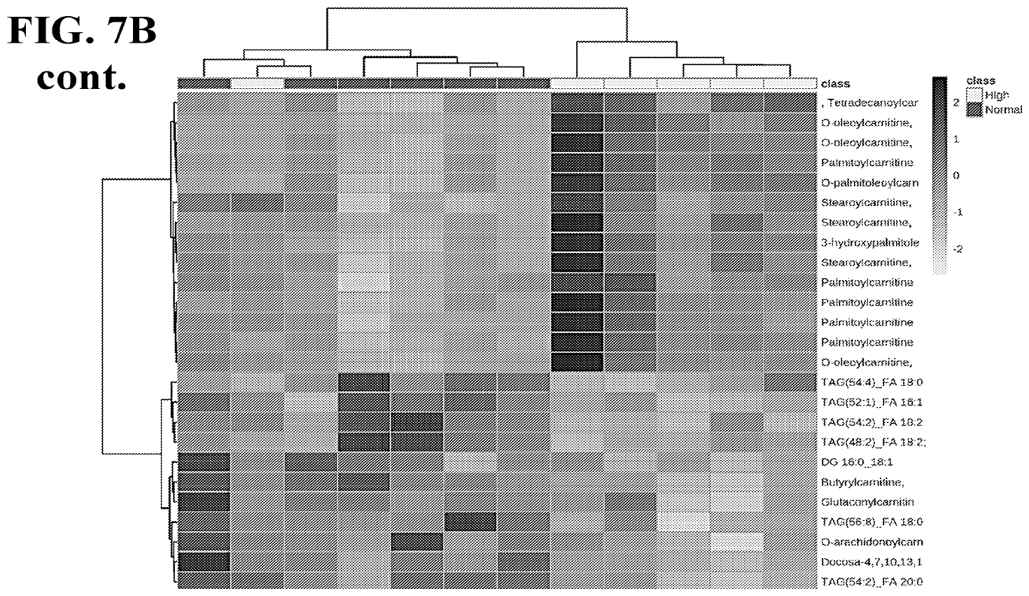

Out of the 1366 ion transitions (MRMs) scanned, 344 had intensities of at least 1.3-fold higher than the blank (see Tables 12 and 13). These compounds were related mainly to PC (79), TAG (74) and AC (66) lipids. The PCA and clustered heatmap analyses revealed distinct clusters between the two pH designations at 30 min postmortem for the lipid method 2, but an overlap was observed for the lipid method 1 (FIGS. 7A and 7B).

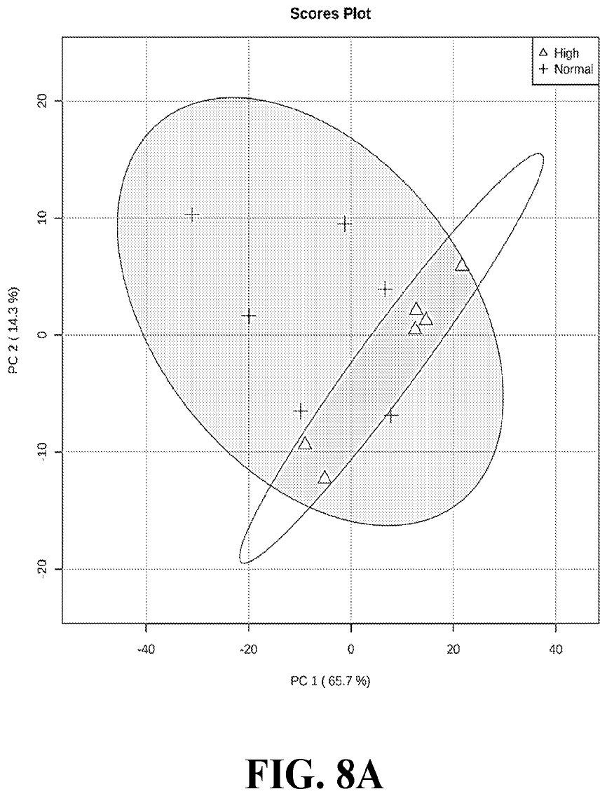

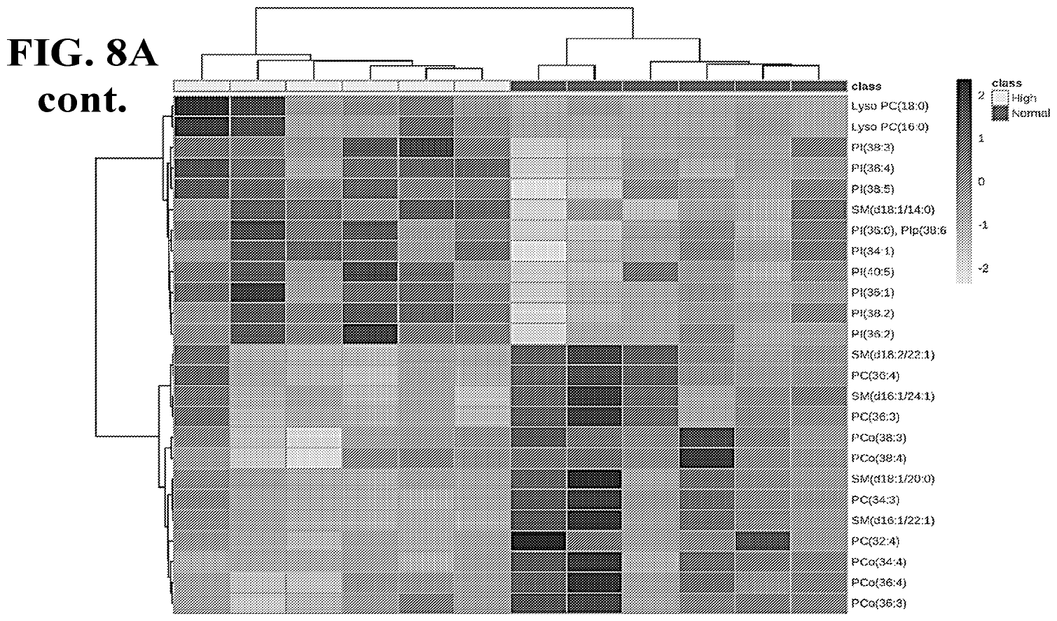

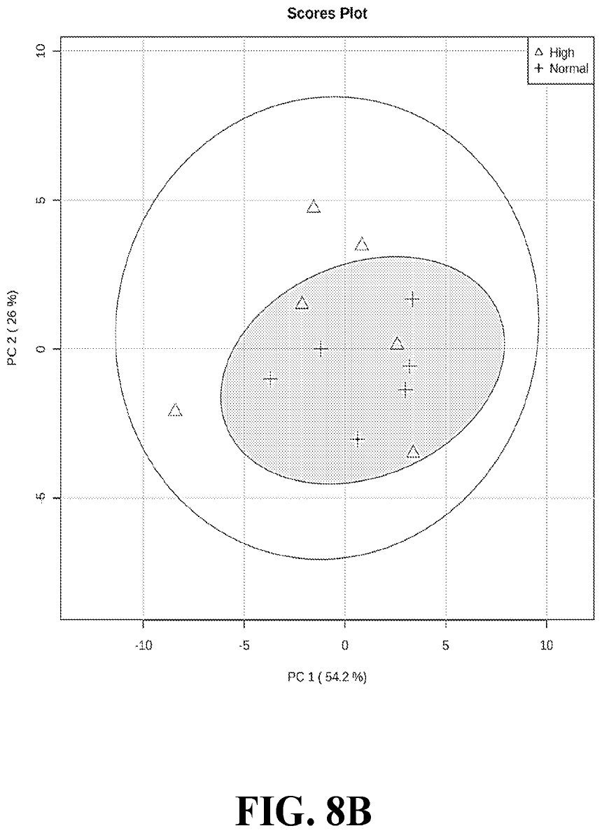

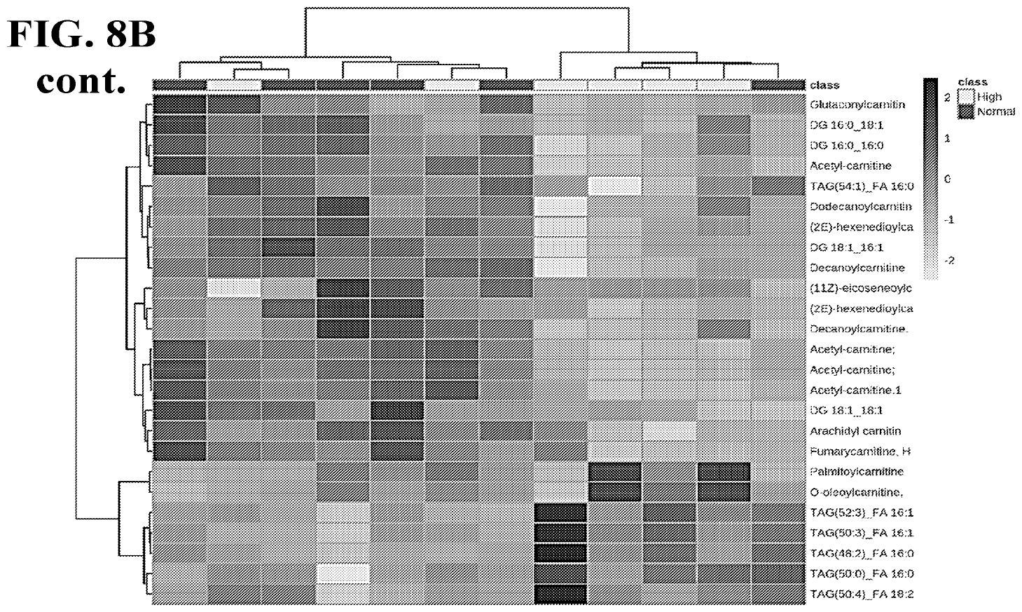

These analyses also revealed distinct clusters due to pHu classification at 44 hour postmortem for the lipid method 1, but an overlap was observed for the lipid method 2 (FIGS. 8A-8B). Relative ion intensity data showed that 14 AC, 5 TAG, 3 PE, 2 PC, and 1 DG differed (P≤0.05) at 30 minutes postmortem between normal and high pHu beef (Table 12). Moreover, 14 PC, 9 PI, 5 SM, 2 AC, and 1 DG differed (P≤0.05) at 44 hours postmortem between pHu classifications (Table 13).

TABLE 12
Lipids that differed (P ≤ 0.05) at 30 minutes postmortem between
Longissiumus thoracis muscle pHu classes.

| | | | |
|---|---|---|---|
| | | Fold | |
| Lipid | Upregulation | ratio^{a} | P-value |

Method 1

| | | | |
|---|---|---|---|
| Lyso PC(18:3) | Normal pHu | 1.7 | 0.026 |
| PC(40:8) | Normal pHu | 1.3 | 0.017 |
| PE(18:2) | Normal pHu | 1.4 | 0.008 |
| PE(36:3) | Normal pHu | 1.3 | 0.010 |
| PE(38:4) | High pHu | 0.8 | 0.034 |

Method 2

| | | | |
|---|---|---|---|
| (5Z)-13-carboxytridec-5-enoylcarnitine | High pHu | 0.7 | 0.013 |
| 3-hydorxypalmitoleoycarnitine | High pHu | 0.7 | 0.018 |
| Butyrylcarnitine | Normal pHu | 1.8 | 0.003 |
| DG 16:0_18:1 | Normal pHu | 1.5 | 0.050 |
| Dococa-4,7,10,13,16-pentaenoyl carnitine | Normal pHu | 1.9 | 0.044 |
| Elaidic carnitine | High pHu | 0.7 | 0.010 |
| Glutaconylcarnitine | Normal pHu | 1.4 | 0.045 |
| Heptadecanoyl carnitine | Normal pHu | 1.3 | 0.018 |
| Hexadecanedioic acid mono-L-carnitine ester | High pHu | 0.6 | 0.035 |
| Isobutyryl- L-carnitine | Normal pHu | 1.8 | 0.003 |
| O-oleoylcarnitine | High pHu | 0.7 | 0.009 |
| O-palmitoleoylcarnitine | High pHu | 0.7 | 0.019 |
| Palmitoylcarnitine | High pHu | 0.7 | 0.013 |
| Stearoylcarnitine | High pHu | 0.6 | 0.030 |
| TAG(48:2)_FA 18:2 | Normal pHu | 1.4 | 0.029 |
| TAG(52:1)_FA 16:1 | Normal pHu | 1.4 | 0.038 |
| TAG(54:2)_FA 18:2 | Normal pHu | 1.5 | 0.019 |
| TAG(54:4)_FA 18:0 | Normal pHu | 1.5 | 0.025 |
| TAG(56:8)_FA 18:0 | Normal pHu | 1.5 | 0.024 |
| Tetradecanoylcarnitine | High pHu | 0.8 | 0.023 |

^{a}Normal pHu/High pHu.

TABLE 13
Lipids that differed (P ≤ 0.05) at 44 hours postmortem
between Longissiumus thoracis muscle pHu classes.

| | | | |
|---|---|---|---|
| | | Fold | |
| Lipid | Upregulation | ratio^{a} | P-value |

Method 1

| | | | |
|---|---|---|---|
| Lyso PC(16:0) | High pHu | 0.4 | 0.023 |
| Lyso PC(18:0) | High pHu | 0.5 | 0.014 |
| PC(32:4) | Normal pHu | 1.4 | 0.023 |
| PC(34:2) | Normal pHu | 1.1 | 0.048 |
| PC(34:3) | Normal pHu | 1.2 | 0.010 |
| PC(34:6) | Normal pHu | 1.1 | 0.045 |
| PC(36:3) | Normal pHu | 1.2 | 0.032 |
| PC(36:4) | Normal pHu | 1.2 | 0.033 |
| PC(38:7) | High pHu | 1.0 | 0.047 |
| PCo(34:4) | Normal pHu | 1.1 | 0.015 |
| PCo(36:3) | Normal pHu | 1.1 | 0.025 |
| PCo(36:4) | Normal pHu | 1.1 | 0.015 |
| PCo(38:3) | Normal pHu | 1.1 | 0.016 |
| PCo(38:4) | Normal pHu | 1.0 | 0.035 |
| PI(34:1) | High pHu | 0.6 | 0.041 |
| PI(36:0) | High pHu | 0.5 | 0.016 |
| PI(36:1) | High pHu | 0.4 | 0.004 |
| PI(36:2) | High pHu | 0.5 | 0.005 |
| PI(38:2) | High pHu | 0.5 | 0.004 |
| PI(38:3) | High pHu | 0.5 | 0.006 |
| PI(38:4) | High pHu | 0.5 | 0.002 |
| PI(38:5) | High pHu | 0.6 | 0.008 |
| PI(40:5) | High pHu | 0.6 | 0.036 |
| SM(d16:1/22:1) | Normal pHu | 1.2 | 0.014 |
| SM(d16:1/24:1) | Normal pHu | 1.2 | 0.026 |
| SM(d18:1/14:0) | High pHu | 0.6 | 0.009 |
| SM(d18:1/20:0) | Normal pHu | 1.1 | 0.043 |
| SM(d18:2/22:1) | Normal pHu | 1.2 | 0.026 |

Method 2

| | | | |
|---|---|---|---|
| (2E)-hexenedioylcarnitine | Normal pHu | 1.4 | 0.037 |
| Arachidyl carnitine | Normal pHu | 1.2 | 0.028 |
| DG 16:0_16:0 | Normal pHu | 1.8 | 0.050 |

^{a}Normal pHu/High pHu.

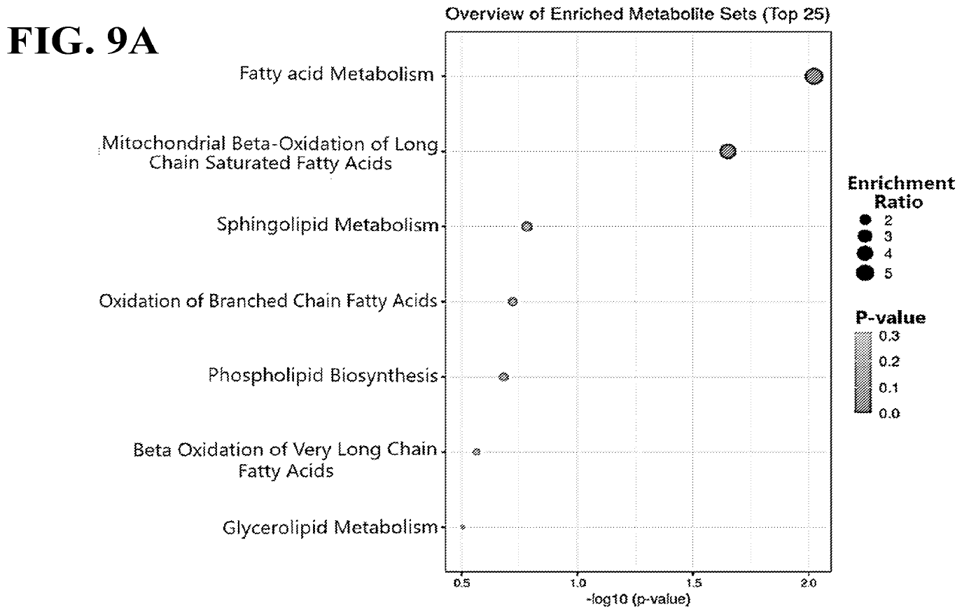

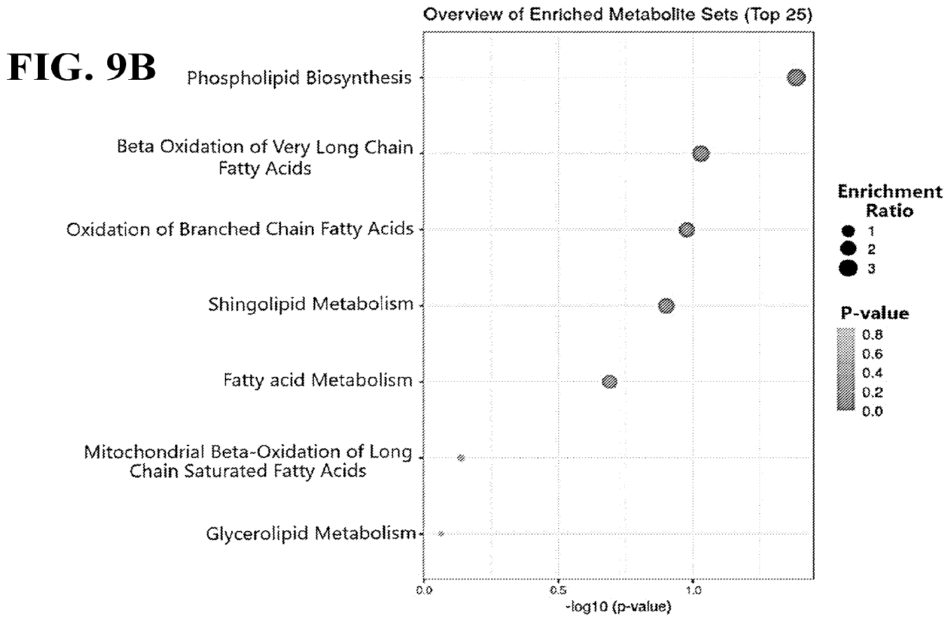

There were no differences (P>0.05) for total TAG and TAG relative amounts according to carbon chain length and degree of unsaturation between normal and high pHu classifications either at 30 minutes or 44 hours postmortem, except that steaks with normal pHu at 30 minutes postmortem contained greater concentrations of 56-carbon (P=0.044), unsaturated (P=0.049) and monounsaturated (P=0.037) TAG compared to that from the high pHu steaks (Table 14). Furthermore, steaks with normal pHu at 44 hours postmortem had more total phospholipids (P=0.042) than those from high pHu steaks (Table 15). No differences in phospholipid classes were noted between normal and high pHu at either 30 minutes or 44 hours postmortem (P>0.05). Quantitative enrichment analyses revealed that the main metabolic pathways affected by pHu classification at 30 minutes postmortem (FIGS. 9A and 9B) were fatty acid metabolism (P=0.009) and mitochondrial betaoxidation of long chain saturated fatty acids (P=0.022). Phospholipid biosynthesis was the main metabolic pathway regulated by pHu class at 44 hours postmortem (P=0.009).

TABLE 14
Means and standard error of mean (SEM) of the effect
of longissimus thoracis muscle pHu classes on triglyceride
profile at different postmortem periods.

| | 30 minutes postmortem | | | 44 hours postmortem | | |
|---|---|---|---|---|---|---|
| | Normal | High | | Normal | High | |
| Item | pHu | pHu | SEM | pHu | pHu | SEM |
| Total triglycerides, | 3.3 | 3.1 | 0.48 | 2.8 | 2.6 | 0.53 |
| ng/μg muscle tissue | | | | | | |

Carbon number, %

| | | | | | | |
|---|---|---|---|---|---|---|
| 48 | 8.8 | 7.3 | 0.57 | 8.7 | 8.5 | 0.73 |
| 50 | 21.1 | 19.8 | 0.56 | 20.1 | 20.8 | 0.49 |
| 52 | 41.8 | 44.4 | 1.49 | 42.0 | 43.7 | 1.77 |
| 54 | 27.7 | 27.8 | 1.16 | 27.9 | 25.9 | 0.90 |
| 56 | 1.1^{a} | 0.7^{b} | 0.13 | 1.3 | 1.1 | 0.28 |

Unsaturation number, %

| | | | | | | |
|---|---|---|---|---|---|---|
| 0 | 6.6^{a} | 4.8^{b} | 0.71 | 6.7 | 6.0 | 0.97 |
| 1 | 27.9^{a} | 25.1^{b} | 0.82 | 26.8^{a} | 23.5^{a} | 0.92 |
| 2 | 44.3 | 49.4 | 1.96 | 43.5 | 47.1 | 2.37 |
| 3 | 15.0 | 15.5 | 0.74 | 14.9 | 16.4 | 0.51 |
| 4 | 4.4 | 3.4 | 0.51 | 4.8 | 4.3 | 0.58 |
| 5 | 0.41 | 0.27 | 0.089 | 0.25 | 0.23 | 0.059 |
| 6 | 0.21 | 0.13 | 0.047 | 0.50 | 0.39 | 0.113 |
| 7 | 0.30 | 0.22 | 0.050 | 0.36 | 0.29 | 0.074 |
| 8 | 2.0 | 1.7 | 0.21 | 2.2 | 1.8 | 0.20 |

Group unsaturation, %

| | | | | | | |
|---|---|---|---|---|---|---|
| Up to 2 | 72.2 | 74.4 | 1.63 | 70.7 | 71.2 | 1.78 |
| >3 | 22.3 | 21.3 | 1.44 | 23.1 | 23.4 | 0.98 |

^{a,b}Values within a row in each postmortem time with different superscripts differ significantly at P ≤ 0.05.

TABLE 15
SEM of the effect of longissimus thoracis muscle pHu classes
on phospholipid profile at different postmortem periods.

| | 30 minutes | | | 44 hours | | |
|---|---|---|---|---|---|---|
| | postmortem | | | postmortem | | |
| Class, ng/μg muscle | Normal | High | | Normal | High | |
| tissue | pHu | pHu | SEM | pHu | pHu | SEM |
| Total phospholipids | 1.03 | 1.05 | 0.167 | 1.28^{a} | 0.99^{a} | 0.100 |
| Phosphatidylcholine | 0.40 | 0.31 | 0.094 | 0.59 | 0.51 | 0.104 |
| Phosphatidyleth- | 0.22 | 0.17 | 0.080 | 0.18 | 0.13 | 0.039 |
| anolamine | | | | | | |
| Phosphatidylglycerol | 0.02 | 0.02 | 0.008 | 0.01 | 0.01 | 0.003 |
| Phosphatidylinositol | 0.09 | 0.10 | 0.033 | 0.06 | 0.04 | 0.013 |
| Sphingomyelin | 0.25 | 0.45 | 0.096 | 0.43 | 0.26 | 0.105 |

^{a,b}Values within a row in each postmortem time with different superscripts differ significantly at P ≤ 0.05.

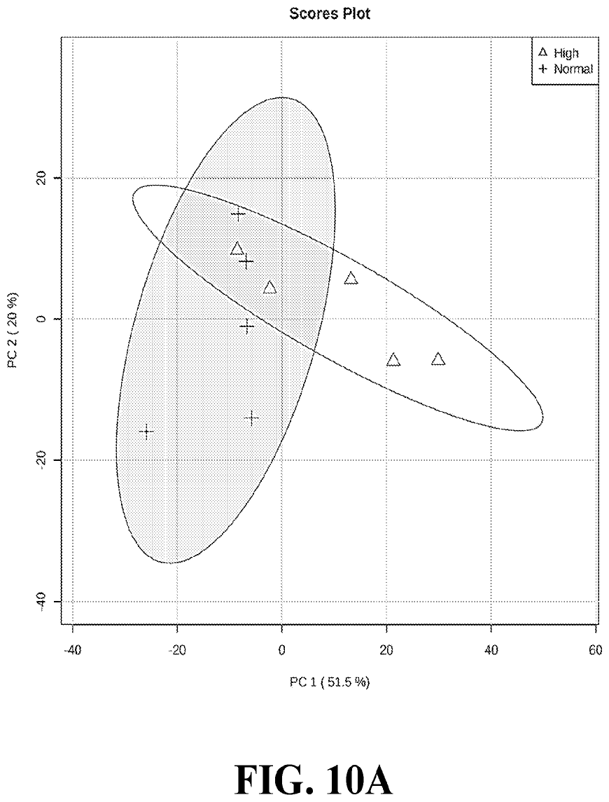

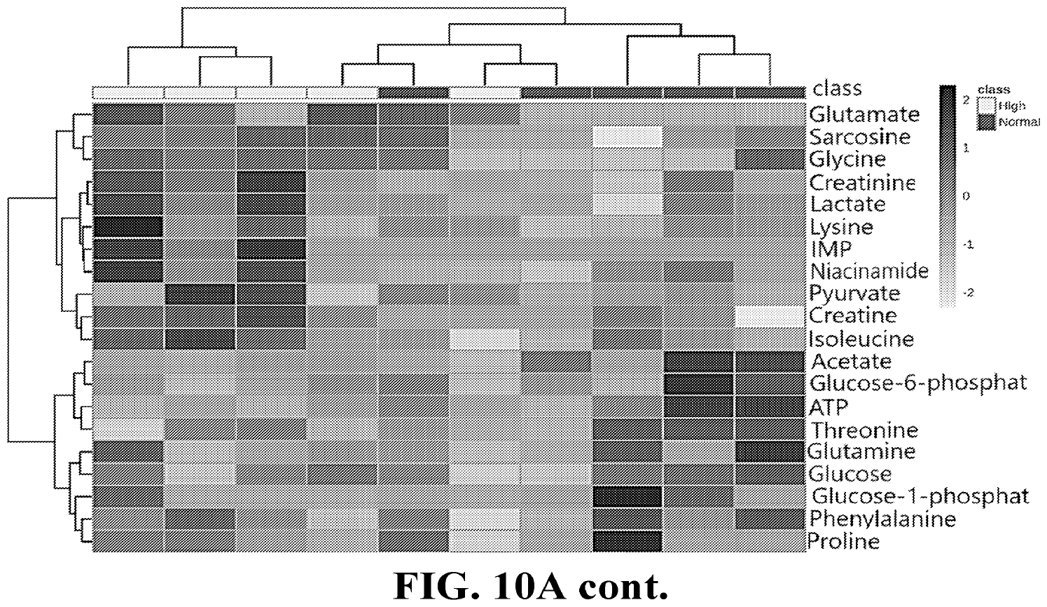

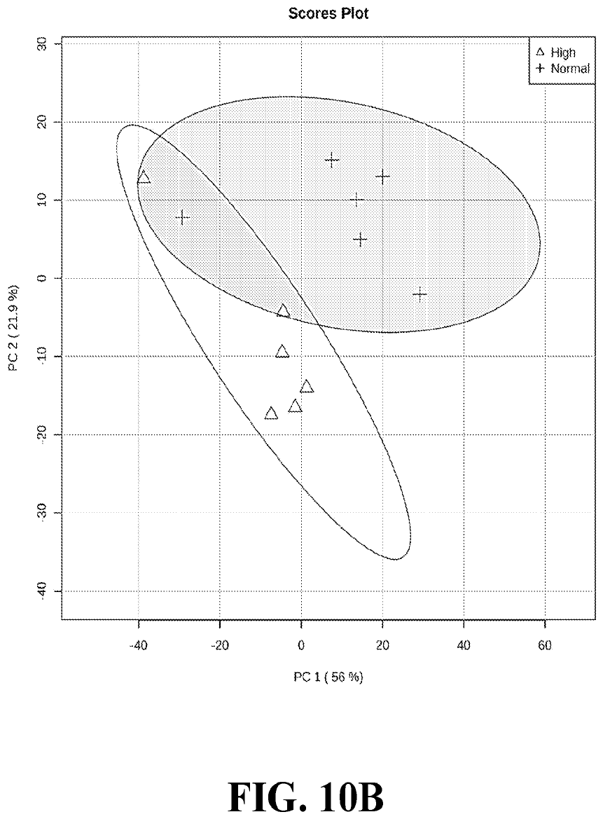

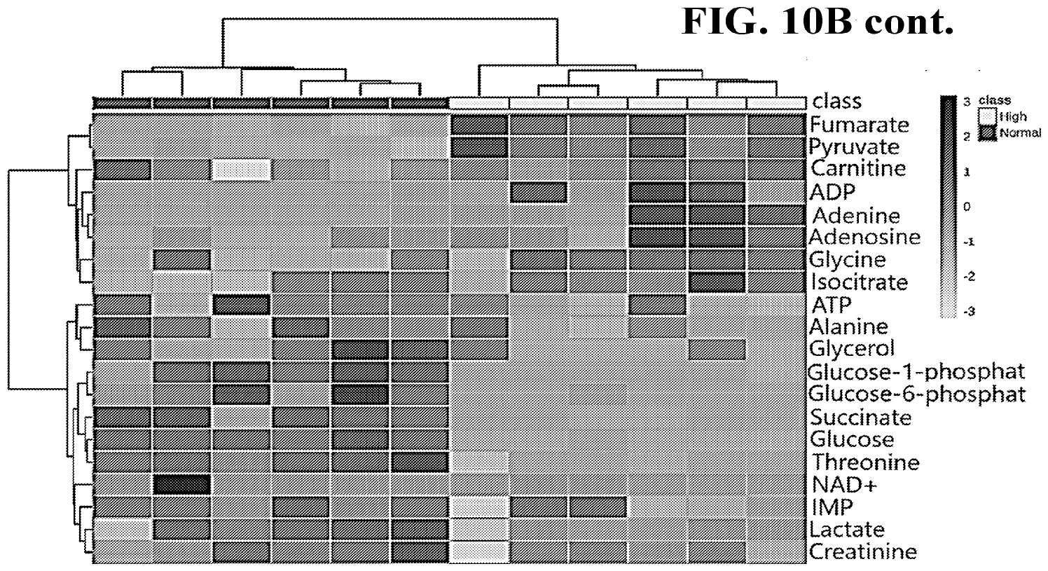

Based on 1D ¹H NMR analyses, 40 metabolites were identified in beef (Table 16). Metabolites were segregated according to pHu class at 30 minutes and 44 hours postmortem (FIGS. 10A and 10B, respectively). Acetate (P=0.047), adenosine triphosphate (ATP) (P=0.045) and glucose-6-phosphate (P=0.075) were higher; and creatine (P=0.075), creatinine (P=0.067), glutamate (P=0.082), and inosine monophosphate (IMP; P=0.076) were lower in normal pHu beef at 30 minutes postmortem that that of high pHu beef.

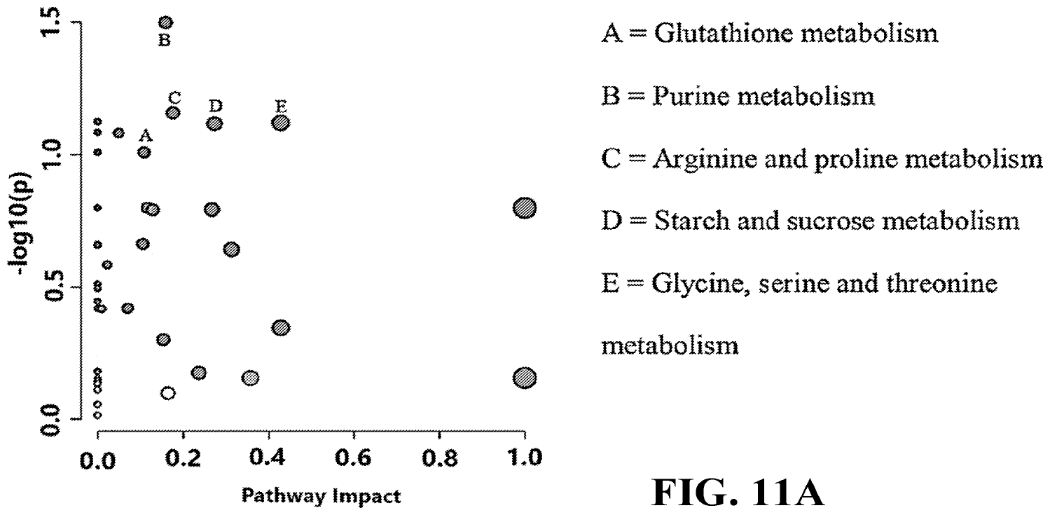

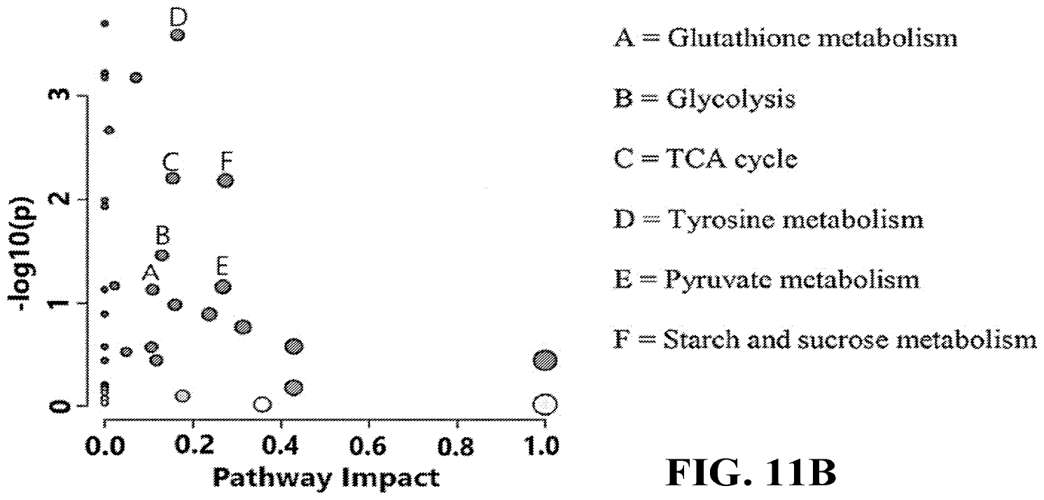

In turn, glucose (P<0.001), glucose-1-phosphate (P=0.007), glucose-6-phosphate (P=0.012), IMP (P=0.086), lactate (P=0.070), succinate (P<0.0051), and threonine (P<0.001) were higher; and adenine (P=0.045), adenosine (P=0.049), adenosine diphosphate (ADP) (P=0.043), fumarate (P<0.001), glycine (P=0.068), and pyruvate (P=0.002) were lower in normal pHu beef at 44 hours postmortem that that of high pHu beef (Table 16). Glutathione (P=0.098; PI=0.11), purine (P=0.032; PI=0.16), arginine and proline (P=0.069; PI=0.18), and glycine, serine and threonine (P=0.076; PI=0.43) metabolisms were the most relevant pathways associated with metabolites detected in normal versus high pHu classifications compared at 30 min h postmortem, while glycolysis (P=0.035; PI=0.13), TCA cycle (P=0.006; PI=0.15), glutathione (P=0.074; PI=0.11), tyrosine (P≤0.001; PI=0.16), and pyruvate (P=0.070; PI=0.27) metabolisms were the most relevant pathways associated with metabolites identified in the normal versus high pHu compared at 44 hours postmortem (FIGS. 11A and 11B).

TABLE 16
Metabolites that differed (P ≤ 0.10) at 30 minutes and 44 hours
postmortem between longissimus thoracis muscle pHu classes.

| | | | |
|---|---|---|---|
| | | Fold | |
| Metabolite | Upregulation | ratio^{a} | P-value |

30 minutes postmortem

| | | | |
|---|---|---|---|
| Acetate | Normal pHu | 1.7 | 0.407 |
| ATP | Normal pHu | 3.9 | 0.045 |
| Creatine | High pHu | 0.6 | 0.075 |
| Glucose-6-phosphate | Normal pHu | 3.2 | 0.075 |
| Glutamate | High pHu | 0.3 | 0.082 |
| IMP | High pHu | <0.1 | 0.076 |

44 hours postmortem

| | | | |
|---|---|---|---|
| Adenine | High pHu | <0.1 | 0.045 |
| Adenosine | High pHu | 0.3 | 0.049 |
| ADP | High pHu | <0.1 | 0.043 |
| Fumarate | High pHu | 0.2 | <0.001 |
| Glucose | Normal pHu | 22.1 | <0.001 |
| Glucose-1-phosphate | Normal pHu | 24.5 | 0.007 |
| Glucose-6-phosphate | Normal pHu | 20.9 | 0.012 |
| Glycine | High pHu | 0.4 | 0.068 |
| IMP | Normal pHu | 1.3 | 0.086 |
| Lactate | Normal pHu | 1.8 | 0.070 |
| Pyruvate | High pHu | 0.3 | 0.002 |
| Succinate | Normal pHu | 65.8 | 0.005 |
| Threonine | Normal pHu | 1.8 | <0.001 |

^{a}Normal pHu/High pHu.

The present study supports that specific TAG and AC were useful in discriminating between high and normal pHu beef at 30 minutes postmortem. Despite lacking differences in total TAG levels at 30 minutes postmortem, decreased amounts of unsaturated, monounsaturated, and 56-carbon TAG were observed in muscle with a high pHu. These results suggest that during early postmortem metabolism, muscle with a high pHu may try to compensate for low glycogen concentrations (48.2±4.41 versus 18.3±2.29 mmol glycogen/kg tissue for normal and high pHu beef, respectively; data not shown) by oxidizing fatty acids to generate ATP for maintaining energy homeostasis in the tissues.

Indeed, muscle postmortem not only uses glycogen but also can use other energy substrates that contribute to ATP production postmortem. Ouali et al. (2013), supra; Scheffler et al., Mitochondria influence postmortem metabolism and pH in an in vitro model, Meat Science 110:118-125 (2015). In fact, evidence gathered to date suggests that high pHu beef can sustain mitochondrial function postmortem, which can use cellular lipids and amino acids to sustain cellular energy demand. Ashmore et al., Respiration of mitochondria isolated from dark-cutting beef, J Animal Science 33 (3): 574-577 (1971); Ouali et al. (2013), supra. More recently, it was reported that mitochondria in muscle retain the capacity to produce ATP aerobically for some time post-exsanguination and, thus, play a key role in maintaining cellular energy status. Ramos et al., Mitochondrial function in oxidative and glycolytic bovine skeletal muscle postmortem, Meat & Muscle Biology 5 (1) (2021).

The quantitative enrichment analysis of the present study supports these constructs and suggests the main metabolic pathways affected by pHu are fatty acid metabolism and mitochondrial beta-oxidation of long chain saturated fatty acids, in line with the study of Gagaoua et al., Dark-cutting beef: A brief review and an integromics meta-analysis at the proteome level to decipher the underlying pathways, Meat Science 181: Article 108611 (2021).

Despite the fact that the most efficient means of generating ATP is through mitochondrial oxidative metabolism, basal concentrations of ATP supply energy for only a few twitches and, therefore, additional mechanisms can buffer energy levels when other metabolic processes are not able to meet tissue metabolic ATP demand. Scheffler et al. (2015), supra; Scheffler & Gerrard (2007), supra. Congruent with this line of reasoning, a higher IMP, creatinine, and creatine content was observed in steaks with a high pHu at 30 minutes postmortem may reflect a compensation of muscle to low glycogen concentrations by increased activity of the phosphagen system, which uses creatine phosphate to regenerate ATP by ADP phosphorylation and creatine formation, whose interconversion is catalyzed by the creatine kinase (CKM).

Higher phosphorylated levels of CKM have been correlated with pH increase in beef and pork as a response to the high energy demands induced by pre-slaughter stress. Mato et al., The first evidence of global meat phosphoproteome changes in response to pre-slaughter stress, BMC Genomics 20 (1): 1-15 (2019); Li et al., Phosphorproteome changes of myofibrillar proteins at early post-mortem time in relation to pork quality as affected by season, J Agricultural & Food Chemistry 63 (47): 10287-10294 (2015). As such, creatinine is likely formed from the degradation of creatine phosphate, while IMP is formed from the deamination of the adenosine monophosphate, which accumulates in the muscle and is unable to contribute to ATP synthesis. Scheffler et al. (2015), supra; Scheffler & Gerrard (2007), supra. The capacity of the phosphagen system to maintain postmortem ATP homeostasis is limited to the availability of creatine phosphate and adenine nucleotides at the time of exsanguination. Once creatine phosphate levels drop, glycolysis becomes the dominant pathway for ATP production. Cassens, Postmortem changes in muscle, Biotechnology & Biotechnological Equipment 4 (5-6): 31-34 (1990).

In the present study, the impact on carbohydrate metabolism, as indicated by a higher glucose-6-phosphate concentration suggests a marked increase in glycolytic capacity or flux in the muscle of steaks with normal pHu during the early postmortem period, which also explains the more abundant concentrations of ATP observed in this group. Ramanathan et al. (2021) and Kiyimba et al. (2022) also reported an increased glycolytic metabolism in steaks with normal pHu compared to that in dark beef, and in turn, darker beef had an increase in oxidative metabolism as indicated by higher mitochondrial content and myoglobin concentrations. Ramanathan et al. (2020), supra; Kiyimba et al., Dark-cutting beef mitochondrial proteomic signatures reveal increased biogenesis proteins and bioenergetics capabilities, J Proteomics: 104637 (2022).

Again, an increased mitochondrial content and function can directly affect mitochondrial oxygen consumption and respiration, thereby affecting myoglobin oxygenation (e.g., lean bloom). Ramanathan et al. (2020), supra; Wu et al., Understanding the development of color and color stability of dark cutting beef based on mitochondrial proteomics, Meat Science 163: Article 108046 (2020). Likewise, Kiyimba et al. (2021) and McKeith et al. (2016) observed an increase in mitochondrial protein content in dark beef compared to that of normal beef with a normal pHu. Kiyimba et al., Changes in glycolytic and mitochondrial protein profiles regulates postmortem muscle acidification and oxygen consumption in dark-cutting beef, J Proteomics 232: Article 104016 (2021); McKeith et al. (2016), supra. Mitochondrial activity gradually decreases with pH decline and normal aging, allowing oxymyoglobin levels to increase, thus causing a much redder beef color. Zhang et al., Characterisation of pH decline and meat color development of beef carcasses during the early postmortem period in a Chinese beef cattle abattoir, J Integrative Agriculture, 17 (7): 1691-1695 (2018).

In support of this, Gagaoua et al. (2018) and Hughes et al. (2014) reported that meat color development is closely related to muscle pH, in which beef with a normal pHu and a gradual pH decline exhibits a constant color increase, while the dark beef with limited pH decline shows minimal color changes postmortem. Gagaoua et al. (2018), supra; Hughes et al., Improving beef meat colour scores at carcass grading, Animal Production Science 54 (4): 422-429 (2014). Similarly, Zhang et al. (2018) reported that beef with a normal pHu becomes lighter and redder with a faster pH decline, while darker beef remains similar in color up to 12 hours postmortem, suggesting it might be possible to predict the development of dark beef early postmortem. Indeed, data from our studies show differences in the muscle lipidome and metabolome were observed as early as 30 min postmortem between normal and high pHu beef and therefore, can be used as indicators of impending beef color defects (decreased L*, a* and b*) and corroborates the notion that these omics may be useful in predicting beef color development.

The present data supports that at 44 hours postmortem, beef metabolites can be useful in discriminating between high from normal pHu beef. Glucose, glucose-1-phosphate, glucose-6-phosphate, and lactate were greater in normal pHu beef, which are indicative of greater glycolysis metabolism. Moreover, initiators of the oxidative metabolism, such as adenosine, ADP, and pyruvate, as well as intermediates of oxidative metabolism (e.g., fumarate), which are most likely mediated by TCA cycle, were greater in beef with a high pHu. Similarly, Cônsolo et al. (2021), supra, showed that most metabolites discriminating between dark and normal-pH beef were associated with cellular energy production pathways, such as: the pentose phosphate pathway, amino and nucleotide sugar metabolism, galactose metabolism and glycolysis. Cônsolo et al. (2021), supra, and Ramanathan et al. (2020), supra, also reported an increased abundance of TCA metabolites, such as fumaric, malic, and citric acid in darker beef. Gagaoua et al. (2021), supra, reported that the greater abundance of TCA metabolites coupled with higher mitochondrial content suggest that darker beef has greater mitochondrial oxygen consumption. As such, increased mitochondrial respiration postmortem may decrease oxygen availability for muscle blooming postmortem and result in darker lean. English et al., Effects of aging on the fundamental color chemistry of darkcutting beef, J Animal Science 94 (9): 4040-4048 (2016); Ramanathan & Mancini (2018), supra.

Moreover, lipids were also useful in discriminating between high from normal pHu beef at 44 hours postmortem, in which some PC and SM were greater in normal pHu beef, while some PI were greater in high pHu beef. Alberts et al. (2002) reported that PI primarily exists in the inner layer of the cell membrane, while PC and SM make up the outer layer of cell membranes. Alberts et al., Molecular biology of the cell, In Annals of Botany (4th ed.), New York, NY: Garland Science (2002). Phosphatidylinositol is a precursor of the phosphoinositide, which is involved in actin rearrangement and calcium regulation across membranes, thus playing an important function in the mitochondrial calcium flux systems. Tolias & Cantley, Pathways for phosphoinositide synthesis. Chemistry & Physics of Lipids 98 (1-2): 69-77 (1999). Therefore, a decrease in some PI concentrations observed in normal pHu beef is likely due to its conversion to phosphoinositide, which acts as an antiapoptotic factor by inhibiting the Bax activation, and consequently inhibits the disruption of the mitochondrial inner membrane potential and the release of cytochrome C and activation of caspase-3. Yamaguchi & Wang, The protein kinase PKB/Akt regulates cell survival and apoptosis by inhibiting Bax conformational change, Oncogene 20 (53): 7779-7786 (2001). This pathway has been proposed to contribute to postmortem proteolysis and meat tenderization via their critical roles in apoptosis. Kemp et al., Tenderness—an enzymatic view, Meat Science 84 (2): 248-256 (2010); Ouali et al. (2013), supra. The role apoptosis signaling in beef tenderization is also supported by the abundance of some SM and PC in normal pHu beef, which are classes of lipids found in animal cell membranes that help prevent damage to the cell structure, and therefore can act as an anti-apoptotic factor. Gault et al. (2010), supra. In this regard, the decrease of some PC concentrations in high pHu beef is likely due to a PC hydrolysis, which is the first stage of postmortem apoptosis and can invert membrane polarity, change membrane fluidity, increase the permeability to ions such as Ca²⁺, augment μ-calpain activity, and thereby increase beef tenderness. Gagaoua et al. (2015), supra. Moreover, PC is also a precursor to cardiolipin, which plays an important role in apoptosis. Mejia & Hatch, Mitochondrial phospholipids: Role in mitochondrial function, J Bioenergetics & Biomembranes 48 (2): 99-112 (2016); Kubli & Gustafsson, Mitochondria and mitophagy: The yin and yang of cell death control, Circulation Research 111 (9): 1208-1221 (2012). Yet, the PC and PI hydrolysis supposed in steaks with high pHu can also indicate a greater phospholipase A activity, which is responsible for releasing fatty acid such as arachidonic acid that acts as a key pro-inflammatory intermediate, promoting oxidative stress. Mavangira & Sordillo, Role of lipid mediators in the regulation of oxidative stress and inflammatory responses in dairy cattle, Research in Veterinary Science, 116 (August 2017): 4-14 (2018). Regardless, these data show that high pHu beef possesses greater oxidative metabolism and thereby may experience higher oxidative stress during the early postmortem, which in turn may increase muscle proteolysis, and consequently contribute to development of more tender beef.

The present study supports that lipid and metabolite profiles indicative of reduced glycolysis and increased use of alternative energy metabolic processes can differentiate high and normal pHu beef. Moreover, assessment of these lipid and metabolite profiles for the biosynthesis of some phospholipids that act in response to cellular stress can also be used to identify high pHu beef (which data supports experiences greater oxidative stress).

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

When ranges are used herein for physical properties, such as molecular weight or pH, all combinations and sub-combinations of ranges and specific embodiments therein are intended to be included, as are the ends of the range specified.

The term “about,” when referring to a number or a numerical range, means that the number or numerical range referred to is an approximation within experimental variability (or within statistical experimental error), and thus the number or numerical range may vary between 1% and 15% of the stated number or numerical range.

The terms and expressions, which have been employed, are used as terms of description and not of limitation. Where certain terms are defined and are otherwise described or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings may be used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered within the scope of the invention as claimed herein.

1. A method of assessing an animal carcass for biomarkers indicative of one or more characteristics of the animal comprising:
obtaining a biomarker profile of a muscle sample from the animal carcass;
comparing the biomarker profile to a representative aggregate biomarker profile of at least a first variation of a first characteristic and a second variation of the first characteristic; and
based on the comparison, assessing the first characteristic of the animal carcass as the first variation or the second variation;
wherein the biomarker profile is a lipidome profile and the representative aggregate biomarker profile is a representative aggregate lipidome profile, or the biomarker profile is a metabolome profile and the representative aggregate biomarker profile is a representative aggregate metabolome profile.

2. The method of claim 1, wherein the animal is poultry, bovine, porcine, ovine, or caprine.

3. The method of claim 1, wherein the one or more characteristics are selected from the group consisting of pH of the muscle sample, gender of the animal, meat tenderness, and finishing regime of the animal.

4. (canceled)

5. The method of claim 1, wherein;
the biomarker profile is a lipidome profile; and
the representative lipidome profile comprises levels of specific diacylglycerols (DG), phosphatidylcholines (PC), triacylglycerols (TG), and sphingomyelins (SM).

6. The method of claim 1, wherein:
the animal is bovine;
the biomarker profile is a lipidome profile;
the first characteristic is finishing regimen;
the first variation of the first characteristic is grass-finished cattle and the second variation of the first characteristic is grain-finished cattle; and
the representative aggregate biomarker profile is a representative aggregate lipidome profile comprising levels of specific DG, PC, TG, and SM.

7. The method of claim 6, wherein the representative aggregate lipidome profile for grass-finished cattle further comprises:
(i) increased levels of PC(32:0), PC(32:2), PC(36:1), PC(36:7), PC(36:8), PC(38:2), PC(32:4), SM(d18:1/22:0), SM(d18:1/24:0), TAG(16:0_36:1), TG (18:1_34:4), and TG (16:1_36:0) as compared to the representative aggregate lipidome profile for grain-finished cattle, and
(ii) decreased levels of DG (18:1_16:0), DG (18:1_18:1), PC(34:2), PC(34:3), PG (36:2), SM(d18:1/20:0), TG (16:0_32:3), and TG (18:0_34:2) as compared to the representative aggregate lipidome profile for grain-finished cattle.

8. The method of claim 5, wherein the DG, PC, TG, and SM comprise:
a total fatty acid chain length of at least 32 carbons, and
up to five sites of unsaturation in fatty acyl chains.

9. The method of claim 8, wherein:
the DG and TG comprise at least one palmitic (16:0) fatty acid, one palmitoleic (16:1) fatty acid, and one oleic (18:1) fatty acid, and
the SM are sphingosine ceramides comprising a d18:1 sphingoid base.

10. The method of claim 6, wherein the representative aggregate lipidome profile for grass-finished cattle further comprises:
a change in the level of one or more other PC, TG, DG and/or SM comprising palmitic (16:0) fatty acid, palmitoleic (16:1) fatty acid, and/or oleic (18:1) fatty acid; and/or
a change in one or more other sphingosine ceramides comprising a d18:1 sphingoid base.

11. The method of claim 1, wherein:
the biomarker profile is a lipidome profile comprising one or more of specific PC, PE, SM, acyl-carnitine (AC) and triacylglycerides (TAG) content levels observed in the muscle sample at about 30 minutes postmortem;
the first characteristic is an ultimate pH (pHu) of the muscle sample; and
the first variation of the first characteristic is muscle with normal pHu and the second variation of the first characteristic is muscle with high pHu.

12. The method of claim 11, wherein:
the representative aggregate biomarker profile for muscle with high pHu comprises decreased levels of one or more of 56-carbon, unsaturated and monosaturated TAG as compared to the representative aggregate biomarker profile for muscle with normal pHu: or
the representative aggregate biomarker profile of muscle with normal pHu comprises increased levels of one or more of PC, PE, SM and AC observed in the muscle at about 30 minutes postmortem as compared to the representative aggregate biomarker profile of muscle with high pHu.

13. (canceled)

14. The method of claim 12, wherein the representative aggregate biomarker profile for muscle with normal pHu comprises increased levels of Lyso PC(18:3), PC(40:8), PE (18:2), PE (36:3), and PE (38:4) as compared to the representative aggregate biomarker profile for muscle with high pHu.

15. The method of claim 1, wherein:
the animal is bovine;
the biomarker profile comprises a metabolome profile and the representative aggregate biomarker profile comprises a representative aggregate metabolome profile;
the first characteristic is pH of the muscle sample; and
the first variation of the first characteristic is muscle with normal pHu and the second variation of the first characteristic is muscle with high pHu.

16. The method of claim 15, wherein the representative aggregate metabolome comprises levels of one or more of:
(a) specific inosine monophosphate (IMP), creatinine, and creatine content observed in muscle at about 30 minutes postmortem, and the representative aggregate metabolome profile for muscle with high pHu comprises increased levels of one or more of IMP, creatinine, and creatine content as compared to the representative aggregate metabolome profile for muscle with normal pHu;
(b) glucose, glucose-1-phosphate, glucose-6-phosphate, and lactate content observed in muscle at about 30 minutes postmortem, and the representative aggregate profile for muscle with high pHu comprises decreased levels of one or more of glucose, glucose-1-phosphate, glucose-6-phosphate, and lactate content as compared to the representative aggregate profile for muscle with normal pHu; and/or
(c) adenosine, ADP, pyruvate, and an intermediate of oxidative metabolism content observed in muscle at about 44 hours postmortem, and the representative aggregate profile for muscle with high pHu comprises increased levels of one or more of adenosine, ADP, pyruvate, and an intermediate of oxidative metabolism content as compared to the representative aggregate profile for muscle with normal pHu.

17. A method of generating a representative aggregate biomarker profile for a first variation of a characteristic and/or a representative aggregate biomarker profile for a second variation of the characteristic, which method comprises:
analyzing, or having analyzed, (a) lipids or other metabolites obtained from muscle samples of a representative aggregate of carcasses of the first variation and/or (b) lipids or other metabolites obtained from muscle samples of a representative aggregate of carcasses of the second variation, wherein the carcasses of both the first variation and the second variation are from the same species of animal;
whereupon a representative aggregate biomarker profile for animals of the first variation and/or a representative aggregate biomarker profile for animals of the second variation is/are generated.

18. The method of claim 17, wherein:
the first variation is grass-finished cattle and the second variation is grain-finished cattle; or
the first variation is meat having normal ultimate pH (pHu) and the second variation is meat having high pHu.

19. The method of claim 17, wherein the first variation is grass-finished cattle and the second variation is grain-finished cattle, and the lipids or other metabolites obtained from muscle samples of a representative aggregate biomarker profile of cattle carcasses of grass-finished cattle comprises levels of diacylglycerols (DG), phosphatidylcholines (PC), triacylgycerols (TG), and sphingomyelins (SM).

20. The method of claim 19, wherein the representative aggregate biomarker profiles of both the first and second variations comprise PC(32:0), PC(32:2), PC(36:1), PC(36:7), PC(36:8), PC(38:2), PC(32:4), SM(d18:1/22:0), SM(d18:1/24:0), TG (16:0_36:1), TAG(18:1_34:4), TG (16:1_36:0), DG (18:1_16:0), DG (18:1_18:1), PC(34:2), PC(34:3), PG (36:2), SM(d18:1/20:0), TG (16:0_32:3), and TG (18:0_34:2).

21. The method of claim 20, wherein the representative aggregate biomarker profiles further comprise one or more other PC, TG, DG and/or SM comprising palmitic (16:0) fatty acid, palmitoleic (16:1) fatty acid, and/or oleic (18:1) fatty acid, and/or one or more other sphingosine ceramides comprising a d18:1 sphingoid base.

22. The method of claim 17, wherein the first variation is meat having normal ultimate pH (pHu) and the second variation is meat having high pHu, and the lipids or other metabolites obtained from muscle samples of a representative aggregate of carcasses for both the first and second variations comprise one or more of specific PC, PE, SM, acyl-carnitine (AC) and triacylglycerides (TAG) content levels observed in the muscle samples at about 30 minutes postmortem.

23. The method of claim 1, further comprising grading the meat at least in part on the assessed first characteristic of the animal carcass.

24-25. (canceled)

26. The method of claim 1, wherein obtaining the biomarker profile of the muscle sample does not require lipid extraction from the muscle sample.

27. A kit for assessing an animal carcass for biomarkers indicative of one or more characteristics of the animal comprising:
a solution for retaining a sample obtained from a muscle of an animal carcass;
a cartridge to deliver a retained sample to a device for analysis that results in a biomarker profile of the muscle sample, wherein the analysis does not require lipid extraction from the retained sample; and
a representative aggregate biomarker profile of at least each of (a) a first variation of a first characteristic, and (b) a second variation of the first characteristic, wherein the representative aggregate biomarker profile is a representative aggregate lipidome profile or a representative aggregate metabolome profile.

28. The kit of claim 27, further comprising a receptacle that houses the solution, wherein the receptacle further comprises an extraction component for extracting the sample from the muscle of the animal.

29. The kit of claim 28, wherein the cartridge comprises a portion configured to receive the solution from the receptacle.

30-31. (canceled)
